SEQUENCE LISTING (44 sequences)

SEQ ID NO: 1 - protein, 5 aa, synthetic construct
NYLIE

SEQ ID NO: 2 - protein, 17 aa, synthetic construct
VTTPGGGTINYAQKFQG

SEQ ID NO: 3 - protein, 13 aa, synthetic construct
SRWDPLYYYALEY

SEQ ID NO: 4 - protein, 122 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSS

SEQ ID NO: 5 - protein, 15 aa, synthetic construct
RASESVDNYGIPFMN

SEQ ID NO: 6 - protein, 7 aa, synthetic construct
AASNRGS

SEQ ID NO: 7 - protein, 9 aa, synthetic construct
QQSEEVPLT

SEQ ID NO: 8 - protein, 111 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIK

SEQ ID NO: 9 - protein, 5 aa, synthetic construct
NYWMT

SEQ ID NO: 10 - protein, 17 aa, synthetic construct
SITYTGGGTYYPDSVKG

SEQ ID NO: 11 - protein, 13 aa, synthetic construct
ANYYYNTYGGFPY

SEQ ID NO: 12 - protein, 122 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSS

SEQ ID NO: 13 - protein, 11 aa, synthetic construct
LASEGFSNDLA

SEQ ID NO: 14 - protein, 7 aa, synthetic construct
AASRLQD

SEQ ID NO: 15 - protein, 9 aa, synthetic construct
QQGSKYPLT

SEQ ID NO: 16 - protein, 107 aa, synthetic construct
DIQMTQSPSSLSASVGDRVTITCLASEGFSNDLAWYQQKPGKSPKLLIYAASRLQDGVPSRFSGSGSGTDFTLTISSLQPEDFATYFCQQGSKYPLTFGGGTKVEIK

SEQ ID NO: 17 - protein, 214 aa, synthetic construct
DIQMTQSPSSLSASVGDRVTITCLASEGFSNDLAWYQQKPGKSPKLLIYAASRLQDGVPSRFSGSGSGTDFTLTISSLQPEDFATYFCQQGSKYPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 451 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 442 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 20 - protein, 229 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 688 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSGGDIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 22 - protein, 451 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 23 - protein, 451 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 442 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 25 - protein, 451 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 26 - protein, 442 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 27 - protein, 229 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 438 aa, synthetic construct
DIQMTQSPSSLSASVGDRVTITCLASEGFSNDLAWYQQKPGKSPKLLIYAASRLQDGVPSRFSGSGSGTDFTLTISSLQPEDFATYFCQQGSKYPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 451 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 30 - protein, 218 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 438 aa, synthetic construct
DIQMTQSPSSLSASVGDRVTITCLASEGFSNDLAWYQQKPGKSPKLLIYAASRLQDGVPSRFSGSGSGTDFTLTISSLQPEDFATYFCQQGSKYPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 451 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 33 - protein, 676 aa, synthetic construct
EVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 467 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGEVQLVESGGGLVQPGGSLRLSCAASGFTFNNYWMTWIRQAPGKGLEWVASITYTGGGTYYPDSVKGRFTISRDDAKSSLYLQMNSLRAEDTAVYYCTRANYYYNTYGGFPYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 35 - protein, 470 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGGGGSGGGGSGGQVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 217 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 37 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 38 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 39 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 107 aa, Homo sapiens [sequence omitted]

SEQ ID NO: 41 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 43 - protein, 122 aa, synthetic construct
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 44 - protein, 107 aa, synthetic construct
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

(19) United States
(12) Patent Application Publication
(10) Pub. No.: US 2026/0209340 A1
(43) Pub. Date: Jul. 23, 2026

(54) TREATMENT OF INFLAMMATORY LUNG DISEASE AND THERAPEUTIC AGENTS USEFUL THEREFOR

(71) Applicant: Hoffmann-La Roche Inc., Little Falls, NJ (US)

(72) Inventors: Sebastian BREUER, Schopfheim (DE); Stefan DENGL, Geretsried (DE); Daniel George LAFKAS, Michelbach-le-Bas (FR); Tilman Sebastian SCHLOTHAUER, Mountain View, CA (US)

(73) Assignee: Hoffmann-La Roche Inc., Little Falls, NJ (US)

(21) Appl. No.: 19/425,173

(22) Filed: Dec. 18, 2025

(30) Foreign Application Priority Data
Dec. 19, 2024 (EP) 24221882.4

(51) Int. Cl. C07K 16/24; A61K 39/00; A61P 11/00; A61P 29/00

(52) U.S. Cl. CPC C07K 16/248; A61P 11/00; A61P 29/00; C07K 16/244; A61K 2039/505; C07K 2317/31; C07K 2317/52; C07K 2317/55; C07K 2317/565; C07K 2317/71; C07K 2317/72; C07K 2317/76; C07K 2317/94

(57) ABSTRACT

The present invention generally relates to the treatment of inflammatory lung disease, particularly chronic obstructive pulmonary disease (COPD), and to therapeutic agents useful therefor. In particular, the present invention relates to antibodies, including bispecific antibodies, useful for the treatment of inflammatory lung disease. In addition, the invention relates to polynucleotides encoding such antibodies, vectors and host cells comprising such polynucleotides, as well as methods for producing such antibodies.

### CROSS-REFERENCE TO RELATED APPLICATIONS

This application claims priority benefit of European Patent Application No. 24221882.4, filed on Dec. 19, 2024, the contents of which are hereby incorporated by reference in their entireties.

### SEQUENCE LISTING

This application contains a Sequence Listing which has been submitted electronically in XML format and is hereby incorporated by reference in its entirety. Said XML copy, created on Dec. 11, 2025, is named “P39753-US-1_Sequence_Listing.xml” and is 48,905 bytes in size.

### FIELD OF THE INVENTION

The present invention generally relates to the treatment of inflammatory lung disease, particularly chronic obstructive pulmonary disease (COPD), and to therapeutic agents useful therefor. In particular, the present invention relates to antibodies, including bispecific antibodies, useful for the treatment of inflammatory lung disease. In addition, the invention relates to polynucleotides encoding such antibodies, vectors and host cells comprising such polynucleotides, as well as methods for producing such antibodies.

### BACKGROUND

Chronic obstructive pulmonary disease (COPD) is a chronic inflammatory lung disease that causes persistent, often progressive airflow obstruction. COPD is typically caused by long-term exposure to irritating gases or particulate matter, most often from cigarette smoke. It is the third leading cause of death worldwide, with more than 300 million patients affected and more than 300 million deaths yearly (2024 GOLD Report, goldcopd.org/2024-gold-report/).

COPD is characterized by respiratory symptoms, including shortness of breath, cough, mucus (sputum) production, wheezing and/or exacerbations. An acute exacerbation is a sudden worsening of symptoms that lasts for several days. Exacerbations are a major driver of disease progression. People with COPD are at increased risk of developing heart disease, lung cancer and a variety of other conditions.

The two most common types of COPD are emphysema (enlargement and destruction of alveoli) and chronic bronchitis (inflammation of the lining of the bronchial tubes), which also often occur together and can vary in severity among individuals with COPD.

COPD is an incurable and progressive disease. Management of COPD includes non-pharmacological measures (e.g. reduction of risk factor exposure, such as smoking cessation) as well as pharmacotherapy of symptoms.

Current treatments include bronchodilators, including LABA (long-acting beta agonists, e.g. salmeterol, formoterol) and LAMA (long-acting muscarinic antagonist, e.g. tiotropium, aclidinium), and/or ICS (inhaled corticosteroids, e.g. fluticasone, budesonide)).

With the recent approval of dupilumab (DUPIXENT™, a monoclonal antibody targeting IL-4Ra), a biologic was added to the arsenal of COPD medications, indicated for “eosinophil high” patients. Further biologics in clinical development for the treatment of COPD include astegolimab (an anti-ST2 antibody), tozorakimab, itepekimab (both anti-IL-33 antibodies) and tezepelumab (an anti-TSLP antibody).

Despite advances in the treatment of COPD, there remains a large unmet need for further, improved treatment options.

### SUMMARY OF THE INVENTION

The lung endothelium plays a pivotal role in maintaining proper lung function both during homeostasis and in response to inflammatory stimuli. During inflammatory responses, it becomes activated to facilitate immune cell recruitment and vascular permeability.

The present inventors have found that IL-33 and IL-6 cooperatively activate endothelial cells, and that combined inhibition of both IL-33 and IL-6 is required to prevent endothelial activation. Similarly, the inventors have found that IL-33 and IL-6 cooperatively cause endothelial barrier dysfunction and increase transmigration of neutrophils through an endothelial layer, and that combined inhibition of IL-33 and IL-6 is necessary for blocking the latter.

Activation and dysfunction of the pulmonary vascular endothelium critically contribute to the pathology of COPD, and are associated with exacerbations of COPD.

Endothelial activation leads to binding of immune cells to the endothelium, and endothelial barrier dysfunction allows increased transendothelial immune cell migration to the lung parenchyma, contributing to tissue inflammation and damage.

By combined inhibition of IL-33 and IL-6, inflammation may be particularly efficiently reduced, thereby improving the treatment of COPD and broadening the responsive patient population. Specifically, the combined inhibition of IL-33 and IL-6 may result in enhanced therapeutic efficacy as compared to that observed with inhibition of an IL-33 inhibitor alone or an IL-6 inhibitor alone.

Accordingly, in a first aspect, the invention provides a method of treating an inflammatory lung disease, particularly chronic obstructive pulmonary disease (COPD), in an individual, comprising administering to the individual a combination of a) an IL-33 inhibitor and b) an IL-6 inhibitor.

In one aspect, the IL-33 inhibitor is an anti-IL-33 antibody or an anti-ST2 (IL33R) antibody. In one aspect, the IL-33 inhibitor is an anti-IL-33 antibody selected from the group consisting of 1E1v8, tozorakimab, itepekimab, etokimab, torudokimab, PF-06817024 and MT-2990. In another aspect, the IL-33 inhibitor is an anti-ST2 antibody selected from the group consisting of astegolimab and GSK3772847. In one aspect, the IL-33 inhibitor is an anti-IL-33 antibody, comprising (i) a heavy chain variable region (VH) comprising the heavy chain complementarity determining region (HCDR) 1 of SEQ ID NO: 9, the HCDR 2 of SEQ ID NO: 10 and the HCDR 3 of SEQ ID NO: 11, and a light chain variable region (VL) comprising the light chain complementarity determining region (LCDR) 1 of SEQ ID NO: 13, the LCDR 2 of SEQ ID NO: 14 and the LCDR 3 of SEQ ID NO: 15; and/or (ii) a VH comprising an amino acid sequence having at least 95%, at least 96%, at least 97%, at least 98%, at least 99% or 100% sequence identity to the VH sequence of SEQ ID NO: 12, and a VL comprising an amino acid sequence having at least 95%, at least 96%, at least 97%, at least 98%, at least 99% or 100% sequence identity to the VL sequence of SEQ ID NO: 16.

In one aspect, the IL-6 inhibitor is an anti-IL-6 antibody or an anti-IL-6R antibody. In one aspect, the IL-6 inhibitor is an anti-IL-6 antibody selected from the group consisting of vamikibart, olokizumab, satralizumab, sirukumab, clazakizumab and siltuximab. In another aspect, the IL-6 inhibitor is an anti-IL-6R antibody selected from the group consisting of tocilizumab and sarilumab. In one aspect, the IL-6 inhibitor is an anti-IL-6 antibody, comprising (i) a heavy chain variable region (VH) comprising the heavy chain complementarity determining region (HCDR) 1 of SEQ ID NO: 1, the HCDR 2 of SEQ ID NO: 2 and the HCDR 3 of SEQ ID NO: 3, and a light chain variable region (VL) comprising the light chain complementarity determining region (LCDR) 1 of SEQ ID NO: 5, the LCDR 2 of SEQ ID NO: 6 and the LCDR 3 of SEQ ID NO: 7; and/or (ii) a VH comprising an amino acid sequence having at least 95%, at least 96%, at least 97%, at least 98%, at least 99% or 100% sequence identity to the VH sequence of SEQ ID NO: 4, and a VL comprising an amino acid sequence having at least 95%, at least 96%, at least 97%, at least 98%, at least 99% or 100% sequence identity to the VL sequence of SEQ ID NO: 8.

In a particular aspect, the IL-33 inhibitor and the IL-6 inhibitor are each an antibody comprised in a multispecific, particularly bispecific, antibody.

In a further aspect, the invention provides, a bispecific antibody that binds IL-33 and IL-6.

In one aspect, the bispecific antibody comprises a first antigen binding domain that binds to IL-6, and a second antigen binding domain that binds to IL-33. In one aspect, the first antigen binding domain comprises (i) a heavy chain variable region (VH) comprising the heavy chain complementarity determining region (HCDR) 1 of SEQ ID NO: 1, the HCDR 2 of SEQ ID NO: 2 and the HCDR 3 of SEQ ID NO: 3, and a light chain variable region (VL) comprising the light chain complementarity determining region (LCDR) 1 of SEQ ID NO: 5, the LCDR 2 of SEQ ID NO: 6 and the LCDR 3 of SEQ ID NO: 7; and/or (ii) a VH comprising an amino acid sequence having at least 95%, at least 96%, at least 97%, at least 98%, at least 99% or 100% sequence identity to the VH sequence of SEQ ID NO: 4, and a VL comprising an amino acid sequence having at least 95%, at least 96%, at least 97%, at least 98%, at least 99% or 100% sequence identity to the VL sequence of SEQ ID NO: 8. In one aspect, the second antigen binding domain comprises (i) a heavy chain variable region (VH) comprising the heavy chain complementarity determining region (HCDR) 1 of SEQ ID NO: 9, the HCDR 2 of SEQ ID NO: 10 and the HCDR 3 of SEQ ID NO: 11, and a light chain variable region (VL) comprising the light chain complementarity determining region (LCDR) 1 of SEQ ID NO: 13, the LCDR 2 of SEQ ID NO: 14 and the LCDR 3 of SEQ ID NO: 15; and/or (ii) a VH comprising an amino acid sequence having at least 95%, at least 96%, at least 97%, at least 98%, at least 99% or 100% sequence identity to the VH sequence of SEQ ID NO: 12, and a VL comprising an amino acid sequence having at least 95%, at least 96%, at least 97%, at least 98%, at least 99% or 100% sequence identity to the VL sequence of SEQ ID NO: 16.

In one aspect, the first antigen binding domain is a Fab molecule wherein the variable domains VL and VH or the constant domains CL and CH1, particularly the variable domains VL and VH, of the Fab light chain and the Fab heavy chain are replaced by each other, and/or the second antigen binding domain is a conventional Fab molecule. In one such aspect, in the constant domain CL of the second antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, the bispecific antibody comprises an Fc domain composed of a first and a second subunit. In one aspect, the Fc domain is an IgG Fc domain, particularly an IgG₁ Fc domain, more particularly a human IgG₁ Fc domain. In one aspect, the Fc domain comprises a modification, particularly an amino acid substitution or combination of amino acid substitutions, promoting the association of the first and the second subunit of the Fc domain. In a further aspect, the Fc domain comprises a modification, particularly an amino acid substitution or combination of amino acid substitutions, that reduces the binding affinity to an Fc receptor and/or effector function of the antibody, as compared to a corresponding antibody comprising an Fc domain without such modification. In still a further aspect, the Fc domain comprises a modification, particularly an amino acid substitution or combination of amino acid substitutions, that increases the binding affinity to an FcRn receptor and/or the serum half-life of the antibody, as compared to a corresponding antibody comprising an Fc domain without such modification.

In one aspect, the first and the second antigen binding domain comprised in the bispecific antibody are each a Fab molecule and are each fused at the C-terminus of the Fab heavy chain to the N-terminus of one of the subunits of the Fc domain. In one aspect, the bispecific antibody is a full-length antibody, particularly a full-length IgG antibody, more particularly a full-length IgG₁ antibody.

According to a further aspect of the invention there is provided an isolated polynucleotide encoding a bispecific antibody of the invention, and a host cell comprising the isolated polynucleotide of the invention.

In another aspect is provided a method of producing a bispecific antibody that binds IL-33 and IL-6, comprising culturing the host cell of the invention under conditions suitable for the expression of the antibody and optionally further comprising recovering the antibody. The invention also encompasses a bispecific antibody that binds IL-33 and IL-6 produced by the method of the invention.

The invention further provides a pharmaceutical composition comprising the bispecific antibody of the invention and a pharmaceutically acceptable carrier.

Also encompassed by the invention are methods of using the bispecific antibody and pharmaceutical composition of the invention. In one aspect the invention provides a bispecific antibody or pharmaceutical composition according to the invention for use as a medicament. In one aspect is provided a bispecific antibody or pharmaceutical composition according to the invention for use in the treatment of a disease, particularly an inflammatory lung disease, more particularly COPD.

Also provided is the use of a bispecific antibody or pharmaceutical composition according to the invention in the manufacture of a medicament, the use of a bispecific antibody or pharmaceutical composition according to the invention in the manufacture of a medicament for the treatment of a disease, particularly an inflammatory lung disease, more particularly COPD.

The invention also provides a method of treating a disease, particularly an inflammatory lung disease, more particularly COPD, in an individual, comprising administering to said individual the bispecific antibody or pharmaceutical composition according to the invention.

Furthermore, the invention provides an IL-33 inhibitor and/or an IL-6 inhibitor for use in the treatment of an inflammatory lung disease according to the method of the invention.

Also provided is the use of an IL-33 inhibitor and/or an IL-6 inhibitor in the manufacture of a medicament for the treatment of an inflammatory lung disease according to the method of the invention.

In a further aspect, the invention also provides a kit comprising a first medicament comprising a IL-33 inhibitor and a second medicament comprising a IL-6 inhibitor, and optionally further comprising a package insert comprising instructions for administration of the first medicament in combination with the second medicament for treating an inflammatory lung disease in an individual according to the method of the invention.

### BRIEF DESCRIPTION OF THE DRAWINGS

FIGS. 1A-IC. IL-33 and IL-6 act cooperatively to cause increased endothelial ICAM-1 levels.

FIG. 1A Stimulation of HUVEC cells with IL-33, IL-6, or the combination for 48 h results in an increase in the percentage of cells expressing surface ICAM-1 with increased concentration as measured by FACS. EC50 values are shown. FIG. 1B Percentage of ICAM-1 expressing HUVEC cells at the highest dose of cytokine stimulation. FIG. 1C AUC calculation of ICAM-1 upregulation curves. n=3 mixed donor lots.

FIGS. 2A-2B IL-33 and IL-6 treatment results in increased immune cell migration through a HUVEC vascular tube. FIG. 2A Composite max intensity projections of live cultures consisting of HUVEC untreated endothelial cells or pretreated for 24 h with IL-6, IL-33, or their combination, perfused with stimulated fluorescently labelled PBMC cells at the end of 48 h co-culture. PBMCs reside inside the vessel and are able to extravasate into the adjacent ECM over time. FIG. 2B Quantification of the number of cells detected in the area of quantification. The data shown are of four technical replicates for each condition.

FIG. 3A Schematic illustration of an 1+1 IgG-like anti-IL-33/anti-IL-6 bispecific antibody, wherein the IL-6 binding domain is a VH/VL crossover Fab molecule, the IL-33 binding domain is a conventional Fab molecule comprising “charge modifications” as described herein (EE=147E, 213E; RK=123R, 124K), and the Fc domain is a human IgG1 Fc domain comprising “knob-into-hole” modifications as described herein. FIGS. 3B-3E Schematic illustration of corresponding antibody heavy and light chains (LC1, HC1, LC2 and HC2, respectively).

FIG. 4A Schematic illustration of an 1+1 IgG-like anti-IL-33/anti-IL-6 bispecific antibody, wherein the IL-33 binding domain is a VH/VL crossover Fab molecule, the IL-6 binding domain is a conventional Fab molecule comprising “charge modifications” as described herein (EE=147E, 213E; RK=123R, 124K), and the Fc domain is a human IgG1 Fc domain comprising “knob-into-hole” modifications as described herein. FIGS. 4B-4E Schematic illustration of corresponding antibody heavy and light chains.

FIG. 5A Schematic illustration of an 1+1 anti-IL-33/anti-IL-6 bispecific antibody with the antigen binding domain fused to the C-terminus of the Fc domain, wherein the IL-6 binding domain is a VH/VL crossover Fab molecule, the IL-33 binding domain is a conventional Fab molecule comprising “charge modifications” as described herein (EE=147E, 213E; RK=123R, 124K), and the Fc domain is a human IgG1 Fc domain comprising “knob-into-hole” modifications as described herein. FIGS. 5B-5E Schematic illustration of corresponding antibody heavy and light chains.

FIG. 6A Schematic illustration of a 2+1 IgG-like anti-IL-33/anti-IL-6 bispecific antibody with a third antigen binding domain fused to the N-terminus of the IgG, wherein the IL-6 binding domain is a VH/VL crossover Fab molecule, the two IL-33 binding domains are each a conventional Fab molecule comprising “charge modifications” as described herein (EE=147E, 213E; RK=123R, 124K), and the Fc domain is a human IgG1 Fc domain comprising “knob-into-hole” modifications as described herein. FIGS. 6B-6E Schematic illustration of corresponding antibody heavy and light chains.

FIG. 7A Schematic illustration of a 2+1 IgG-like anti-IL-33/anti-IL-6 bispecific antibody with a third antigen binding domain fused to the C-terminus of the IgG, wherein the IL-6 binding domain is a VH/VL crossover Fab molecule, the two IL-33 binding domains are each a conventional Fab molecule comprising “charge modifications” as described herein (EE=147E, 213E; RK=123R, 124K), and the Fc domain is a human IgG1 Fc domain comprising “knob-into-hole” modifications as described herein. FIGS. 7B-7E Schematic illustration of corresponding antibody heavy and light chains.

FIGS. 8A-8C. Combining anti-IL-6 and anti-IL-33 antibodies into bispecific formats maintains the potency of the parental monoclonal antibodies. FIG. 8A Non-targeting control and blocking antibodies were incubated at a dose range of 0.004-100 nM with 1 ng/ml of hIL-6 for 45 minutes prior to the addition of HEK-BLUE™ IL-6 cells. After a 24 h incubation, secreted embryonic alkaline phosphatase (SEAP) activity was assessed using QUANTI-BLUE™ and optical density at 620 nM (OD 620 nM) values were plotted. FIGS. 8B-8C Non-targeting control and blocking antibodies were incubated at a dose range of 0.004-100 nM with 1 ng/ml of hIL-33_wt or hIL-33_mut for 45 minutes prior to the addition of HEK-BLUE™ IL-33 cells. After a 24 h incubation, SEAP activity was assessed using QUANTI-Blue™ and optical density at 620 nM (OD 620 nM) values were plotted.

FIGS. 9A-9D. IL-33 and IL-6 cause endothelial activation through non-redundant pathways. FIG. 9A ICAM-1 levels were measured by FACS on HUVEC cells treated with 4 ng/ml IL-33 and 11 ng/ml IL-6+22 ng/ml sIL-6R for 48 h, and simultaneously with blocking (IgG₁) antibodies against IL-33, IL-6 or the combination of the two. P1AI5003=anti-IL-33 IgG1 antibody with the VH and VL sequences of SEQ ID NOs 43 and 44, respectively (and the PG LALA Fc mutations); P1AE1999=anti-IL-6 Fab molecule with the VH and VL sequences of SEQ ID NOs 4 and 8, respectively; P1AL9289=anti-IL-33 Fab molecule with the VH and VL sequences of SEQ ID NOs 12 and 16, respectively. FIG. 9B Bar graph of ICAM-1 inhibition at the highest dose of antibodies. n=1 donor pool. FIG. 9C Similarly as in FIG. 9A, when control non-targeting or blocking antibodies against IL-33 or IL-6, or bispecific antibodies targeting both IL-33 and IL-6 (in 1+1 and 2+1 format) were used, only the treatment with the bispecific antibodies was able to completely prevent HUVEC activation. FIG. 9D Bar graph of ICAM-1 inhibition at the highest dose of antibodies. n=3 donor pools.

FIGS. 10A-10H Bulk mRNA sequencing of HUVEC treated with IL-33, IL-6/sIL-6R, or IL-33+IL-6/sIL-6R for 24 h. FIG. 10A Principal Component Analysis (PCA) plots generated using the 500 most highly variable genes between treatment conditions and vehicle control. FIG. 10B Venn diagram of differentially expressed genes (DEGs) between each individual cytokine treatment compared to the vehicle control group. Numbers indicate the number of DEGs, and the percentages indicate the percentage of those DEGs over all DEGs included in the Venn diagram. FIGS. 10C-10E Pathway analysis using the Gene Ontology (GO) term “Biological Process” and the DEGs between each individual cytokine treatment group compared to the vehicle control group. FIGS. 10F-10H Expression levels of the three adhesion molecule genes ICAM-1, SELE, SELL for each donor pool of HUVECs. n=2 donor pools and four technical replicates for each.

FIGS. 11A-11E Neutrophil migration through a HUVEC vascular tube and inhibition by therapeutic antibodies. FIG. 11A Representative images of the microfluidic system and neutrophils migrating through the endothelial layer into the extracellular matrix (ECM) towards a gradient of CXCL8 treated with IL-33, IL-6/sIL-6R, or IL-33+IL-6/sIL-6R (referred to as combo), with and without blocking antibodies. The endothelial layer is stained with phalloidin, while neutrophils are stained with anti-elastase antibody and appear as bright puncta. FIG. 11B Bar graph showing the number of cells that migrated over 24 h into the ECM under different cytokine treatment conditions, without and with blocking antibody co-treatment. n=1 HUVEC donor pool and n=4 neutrophil donors. FIG. 11C Levels of CCL2 protein measured in the media collected from the endothelial tubule lumen at the end of the experiment. *=p<0.05, **=p<0.01. FIGS. 11D-11E Bar graph showing the number of cells that migrated over 24 h into the ECM under IL-33+IL-6/sIL-6R (referred to as combo) treatment conditions and an anti-IL-33/anti-IL-6 bispecific antibody (BsAb) or the combination of the monospecific antibodies (anti-IL-33, anti-IL-6). For the protective setting, antibodies were premixed with the cytokines prior to treatment. For the recovery setting, antibodies were added approximately 16 hours after the cytokine treatment. n=1 HUVEC donor pool, n=2 neutrophil donors, and four technical replicates for each graph. *=p<0.05, **=p<0.01, ***=p<0.001.

### DETAILED DESCRIPTION OF THE INVENTION

### Definitions

Terms are used herein as generally used in the art, unless otherwise defined in the following.

As used herein, the terms “first”, “second” or “third” with respect to antigen binding domains etc., are used for convenience of distinguishing when there is more than one of each type of moiety. Use of these terms is not intended to confer a specific order or orientation of the moiety unless explicitly so stated.

The terms “anti-[XXX] antibody” (e.g. “anti-IL-6 antibody”, “anti-IL-33 antibody” etc.) and “an antibody that binds to [XXX]” (e.g. “an antibody that binds to IL-6”, “an antibody that binds to “IL-33” etc.) refer to an antibody that is capable of binding [XXX] with sufficient affinity such that the antibody is useful as a diagnostic and/or therapeutic agent in targeting [XXX]. In some aspects, the extent of binding of an anti-[XXX] antibody to an unrelated, non-[XXX] protein is less than about 10% of the binding of the antibody to [XXX] as measured, e.g., by surface plasmon resonance (SPR). In certain aspects, an antibody that binds to [XXX] has a dissociation constant (K_{D}) of ≤1 nM, ≤500 pM, ≤200 pM, ≤100 pM, ≤10 pM, ≤1 pM, ≤0.5 pM, ≤0.2 pM, or ≤0.1 pM, particularly a K_{D} of ≤1 pM, more particularly a K_{D} of ≤0.5 pM, as measured by SPR at 25° C.

By “specific binding” is meant that the binding is selective for the antigen and can be discriminated from unwanted or non-specific interactions. Suitable assays for determining the specificity of an antibody are known in the art. In some aspects, the extent of binding of an antibody to an unrelated protein is less than about 10% of the binding of the antibody to the antigen as measured, e.g., by SPR.

The term “antibody” encompasses various antibody structures exhibiting the desired antigen-binding activity, including but not limited to monoclonal antibodies, polyclonal antibodies, multispecific antibodies (e.g. bispecific antibodies), and antibody fragments.

An “antibody fragment” refers to a molecule other than a full-length antibody that comprises a portion of a full-length antibody that binds the antigen to which the full-length antibody binds. Examples of antibody fragments include but are not limited to Fv molecules, Fab molecules, Fab′ molecules, Fab′-SH molecules, F(ab′)₂ molecules, diabodies, linear antibodies, single-chain antibody molecules (e.g. scFv and scFab molecules), single-domain antibody molecules, and multispecific (e.g. bispecific) antibodies formed from antibody fragments. For a review of certain antibody fragments, see Hollinger and Hudson, Nature Biotechnology 23:1126-1136 (2005).

The term “full-length antibody” refers to an antibody having the structure of an immunoglobulin molecule comprising two light chains and two heavy chains, and comprising an Fc domain as defined herein. In one aspect, the antibody is a full-length IgG₁ antibody.

The term “monoclonal antibody” as used herein refers to an antibody obtained from a population of substantially homogeneous antibodies, i.e. the antibodies forming this population are essentially identical, except for possible post-translational modifications arising e.g. during manufacturing and/or storage. These antibodies are directed against the same epitope (or the same group of epitopes in the case of multispecific monoclonal antibodies, e.g. the same pair of epitopes in the case of bispecific monoclonal antibodies). This definition expressly excludes polyclonal antibody preparations which are mixtures of antibodies directed against different epitopes. Monoclonal antibodies in accordance with the present invention may be made by a variety of techniques, including but not limited to hybridoma methodology, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment. In one aspect, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC, affinity chromatography, size exclusion chromatography) methods. For review of methods for assessment of antibody purity, see, e.g., Flatman et al., J. Chromatogr. B 848:79-87 (2007). In one aspect, the antibody provided by the present invention is an isolated antibody.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In one aspect, a human antibody is derived from a non-human transgenic mammal, for example a mouse, a rat, or a rabbit. In one aspect, a human antibody is derived from a hybridoma cell line. Antibodies or antibody fragments isolated from human antibody libraries are also considered human antibodies or human antibody fragments herein.

A “humanized” antibody refers to an antibody comprising amino acid residues from non-human CDRs and amino acid residues from human FRs. In one aspect, a humanized antibody will comprise substantially all of at least one, and typically two, variable domains, in which all or substantially all of the CDRs correspond to those of a non-human antibody, and all or substantially all of the FRs correspond to those of a human antibody. One or more FR residues in a humanized antibody may be substituted with corresponding residues from a non-human antibody (e.g., the antibody from which the CDR residues are derived), e.g., to restore or improve antibody specificity or affinity. Such variable domains are referred to herein as “humanized variable region”. A humanized antibody optionally may comprise at least a portion of an antibody constant region derived from a human antibody. A “humanized form” of an antibody, e.g., a non-human antibody, refers to an antibody that has undergone humanization. Typically, a non-human antibody is humanized to reduce immunogenicity to humans, while retaining the specificity and affinity of the parental non-human antibody.

The term “antigen binding domain” refers to the part of an antibody that comprises the area which binds to and is complementary to part or all of an antigen. An antigen binding domain may be provided by, for example, one or more antibody variable domains (also called antibody variable regions). In a preferred aspect, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and complementarity determining regions (CDRs). See, e.g., Kindt et al., Kuby Immunology, 6^{th} ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Glutamine or glutamate residues at the N-terminus of antibody heavy or light chains may be converted to pyro-glutamate spontaneously (see e.g. Liu et al., Journal of Pharmaceutical Sciences 97, 2426-2447 (2008), Rehder et al., Journal of Chromatography A 1102, 164-175 (2006), Chelius et al., Anal Chem 78, 2370-2376 (2006)). Hence, variable regions or variable domains disclosed herein which comprise either a glutamine (Q) or a glutamate (E) amino acid residue at the N-terminus of an the antibody heavy or light chain, may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N terminal pyro-glutamate (pyroE) residue instead of the N-terminal Q or E residue. Accordingly, for each antibody heavy chain, light chain, or variable domain or region sequence disclosed herein that contains an N-terminal Q or E residue, the corresponding sequence with an N-terminal pyroE residue is also encompassed.

Unless otherwise indicated, CDR residues and other residues in the variable domain (e.g., FR residues) are numbered herein according to Kabat et al., Sequences of Proteins of Immunological Interest, 5th Ed. Public Health Service, National Institutes of Health, Bethesda, MD (1991).

The term “complementarity determining region” or “CDR” as used herein refers to each of the regions of an antibody variable domain which are hypervariable in sequence and which determine antigen binding specificity. Generally, antibodies comprise six CDRs: three in the VH (HCDR1, HCDR2, HCDR3), and three in the VL (LCDR1, LCDR2, LCDR3). CDRs are defined by a variety of methods/systems by those skilled in the art. These systems and/or definitions have been developed and refined over a number of years and include Kabat, Chothia, I/MT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. Software programs (e.g., abYsis: world wide web.abysis.org/abysis/sequence_input/key_annotation/key-annotation.cgi) are available and known to those of skill in the art for analysis of antibody sequences and determination of CDRs.

Exemplary CDRs herein include (numbering of amino acid residues according to the reference cited, i.e. Chothia numbering for the Chothia and Contact definition, Kabat numbering for the Kabat definition and IMGT numbering for the IMGT definition):
(a) hypervariable loops occurring at amino acid residues 26-32 (L1), 50-52 (L2), 91-96 (L3), 26-32 (H1), 53-55 (112), and 96-101 (113), according to Chothia and Lesk, J. Mol. Biol. 196:901-917 (1987) (“Chothia definition”);
(b) CDRs occurring at amino acid residues 24-34 (L1), 50-56 (L2), 89-97 (L3), 31-35bB (H1), 50-65 (112), and 95-102 (113), according to Kabat et al., Sequences of Proteins of Immunological Interest, 5th Ed. Public Health Service, National Institutes of Health, Bethesda, MD (1991) (“Kabat definition”);
(c) antigen contacts occurring at amino acid residues 3027c-36 (L1), 46-55 (L2), 89-96 (L3), 30-35b (H1), 47-58 (112), and 93-101 (113), according to MacCallum et al. J. Mol. Biol. 262: 732-745 (1996) (“Contact definition”); and
(d) CDRs occurring at amino acid residues residues 27-38 (L1), 56-65 (L2), 105-117 (L3), 27-38 (H1), 56-65 (H2), and 105-117 (H3), according to Lefranc et al. Dev. Comp. Immunol. 27: 55-77 (2003) (“IMGT definition”).

Unless otherwise indicated, the CDRs are determined herein according to Kabat et al., supra. One of skill in the art will understand that the CDR designations can also be determined according to Chothia, supra, MacCallum, supra, Lefranc, supra, or any other scientifically accepted definition/system. “Framework” or “FR” refers to variable domain residues other than complementarity determining regions (CDRs). The FR of a variable domain generally consists of four FR domains: FR1, FR2, FR3, and FR4. Accordingly, the CDR and FR sequences generally appear in the following order in VH (or VL): FR1-HCDR1(LCDR1)-FR2-HCDR2(LCDR2)-FR3-HCDR3(LCDR3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes is 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3.

The terms “constant region derived from human origin” or “human constant region” as used herein denotes a constant region of a human antibody, in particular a heavy chain constant region of a human antibody of the subclass IgG₁, IgG₂, IgG₃, or IgG₄ and/or a light chain kappa or lambda constant region. Such constant regions are well known in the state of the art and e.g. described by Kabat, E. A., et al., Sequences of Proteins of Immunological Interest, 5th ed., Public Health Service, National Institutes of Health, Bethesda, MD (1991). Unless otherwise specified herein, numbering of amino acid residues in the constant region is according to the numbering system as described in Kabat, E. A. et al., Sequences of Proteins of Immunological Interest, 5th ed., Public Health Service, National Institutes of Health, Bethesda, MD (1991), NIH Publication 91-3242. Specifically, the Kabat numbering system (referred to as “numbering according to Kabat” or “Kabat numbering” herein; see pages 647-660 of Kabat et al., supra) is used for the light chain constant domain of kappa and lambda isotype, and the Kabat EU index numbering system (referred to as “numbering according to Kabat EU index”, “Kabat EU index numbering” or “Kabat EU numbering” herein, see pages 661-723 of Kabat et al., supra) is used for the heavy chain constant domains.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types (or classes), called α (IgA), δ (IgD), ε (IgE), γ (IgG), or μ (IgM), some of which may be further divided into subtypes (or subclasses), e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The “class” of an antibody or immunoglobulin refers to the type of constant domain or constant region possessed by its heavy chain. There are five major classes of antibodies: IgA, IgD, IgE, IgG, and IgM. The heavy chain constant domains that correspond to the different classes of immunoglobulins are called α, δ, ε, γ, and μ, respectively. Several of the antibody classes may be further divided into subclasses (isotypes), e.g., IgG₁, IgG₂, IgG₃, IgG₄, IgA₁, and IgA₂, with corresponding heavy chain constant domains γ₁ (IgG₁), γ₂ (IgG₂), γ₃ (IgG₃), γ₄ (IgG₄), α₁ (IgA₁) and α₂ (IgA₂). The light chain of an antibody may be assigned to one of two types, called kappa (κ) and lambda (λ), based on the amino acid sequence of its constant domain.

A “Fab molecule” or “Fab fragment” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 (CH1) is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain (VH) is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “Fc domain” or “Fc region” (used interchangeably) herein is used to define a C-terminal region of an immunoglobulin heavy chain that contains at least a portion of the constant region.

The term includes native sequence Fc domains and variant Fc domains. In one aspect, a human IgG heavy chain Fc domain extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case in particular where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc domain may or may not be present. Amino acid sequences of heavy chains including an Fc domain (or a subunit of an Fc domain as defined herein) are denoted herein without C-terminal lysine if not indicated otherwise. The corresponding sequence including a C-terminal lysine residue is also encompassed, however. Accordingly, in one aspect, a heavy chain including an Fc domain (subunit) as specified herein comprises an additional C-terminal lysine residue (K447, Kabat EU numbering). Also encompassed is the corresponding sequence without the C-terminal glycine residue. Accordingly, in one aspect, a heavy chain including an Fc domain (subunit) as specified herein lacks the C-terminal glycine residue (G446, Kabat EU numbering). In such a heavy chain, the C-terminal amino acid residue may be proline (P445, Kabat EU numbering) or proline amide (P445-NH₂, Kabat EU numbering). Unless otherwise specified herein, numbering of amino acid residues in the Fc domain or heavy chain constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

A “multispecific antibody” is one having binding specificities for at least two different epitopes, i.e., different epitopes on different antigens or different epitopes on the same antigen. In one aspect, a multispecific antibody is a “bispecific antibody” having binding specificities for two epitopes. Typically, a bispecific antibody comprises two antigen binding sites, each of which is specific for a different epitope. In one aspect, the multispecific antibody has three or more binding specificities. Multispecific antibodies (e.g. bispecific antibodies) may comprise antibody fragments and/or one or more Fc domain.

The term “valent” as used herein denotes the presence of a specified number of antigen binding sites in an antibody. As such, e.g. the term “monovalent” denotes the presence of one (and not more than one) antigen binding site specific for the antigen in the antibody.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

“IL-6” (interleukin-6) refers to human IL-6. The amino acid sequence of human IL-6 is shown in UniProt entry no. P05231 (sequence version 1) and reproduced as SEQ ID NO: 37 (without the N-terminal signal sequence) herein.

“IL-6R” (interleukin-6 receptor) refers to the human IL-6R alpha and/or beta subunits. The amino acid sequences of human IL-6R alpha and human IL-6R beta are shown in UniProt entry no. P08887 (sequence version 1) and UniProt entry no. P40189 (sequence version 2), respectively.

“IL-33” (interleukin-33) refers to human IL-33. The amino acid sequence of full-length (amino acids 1-270) human IL-33 is shown in UniProt entry no. 095760 (sequence version 1) and reproduced as SEQ ID NO: 38 herein. The term “IL-33” as used herein also encompasses C-terminal peptides resulting from proteolytic processing of IL-33, specifically peptides consisting of amino acids 95-270, 99-270 or 109-270 of human IL-33.

“ST2” (interleukin-1 receptor-like 1 (ILRL1), receptor for interleukin-33) refers to human ST2. The amino acid sequence of human ST2 is shown in UniProt entry no. Q01638 (sequence version 4).

By “IL-6 inhibitor” or “IL-33 inhibitor” is meant a molecule that inhibits the respective cytokine signaling pathway. Specifically, an “IL-6 inhibitor” or “IL-33 inhibitor” may be a molecule that inhibits the interaction of IL-6 with its receptor (IL-6R) or IL-33 with its receptor (ST2), respectively. Such molecule may be a chemical compound or a biological, e.g a protein- or nucleotide-based molecule. In particular aspects, the IL-6 inhibitor and/or IL-33 inhibitor is an antibody, specifically a blocking antibody.

A “blocking” antibody herein denotes an antibody that interferes with the function of its target, e.g. by preventing other molecules (e.g. a receptor of the target molecule) from interacting with that target.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (K_{D}). Affinity can be measured by well-established methods known in the art. A preferred method for measuring affinity is Surface Plasmon Resonance (SPR).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In some aspects, the modification promoting the association of the first and the second subunit of the Fc domain comprises an amino acid mutation in the Fc domain, specifically an amino acid substitution. In a preferred aspect, the modification promoting the association of the first and the second subunit of the Fc domain comprises a separate amino acid mutation, specifically an amino acid substitution, in each of the two subunits of the Fc domain.

The term “effector functions” refers to those biological activities attributable to the Fc domain of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B-cell receptor), and B-cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc domain specifically bind, generally via the protein part that is N-terminal to the Fc domain. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

As used herein, the terms “engineer, engineered, engineering”, are considered to include any manipulation of the peptide backbone or the post-translational modifications of a naturally occurring or recombinant polypeptide or fragment thereof Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc domain, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-258), and Pearson et. al. (Genomics 46 (1997) 24-36) and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “polynucleotide” or “nucleic acid molecule” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al. (2017) Nature Medicine 23:815-817, or EP 2101823 B1).

An “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that is different from its natural chromosomal location.

“Isolated polynucleotide (or nucleic acid) encoding an antibody” refers to one or more polynucleotide molecules encoding antibody heavy and light chains (or fragments thereof), including such polynucleotide molecule(s) in a single vector or separate vectors, and such polynucleotide molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Suitable host cells may include, for example, CHO cells, HFK-293 cells, Expi293F cells, PER.C6 cells, NSO cells, lymphocytic cells, prokaryotic cells such as E. coli, and other eukaryotic hosts such as plant cells and fungi. Human host cells are included with the proviso that they are not used within the human body. In one aspect, the host cell of the invention is a eukaryotic cell, particularly a mammalian cell. In one aspect, the host cell is not a cell within a human body.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the composition would be administered. Specifically, the term refers to a preparation of the antibody of the invention and one or more pharmaceutically acceptable carriers or excipients.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, surfactant and/or preservative.

As used herein, “treatment” (and grammatical variations thereof such as “treat” or “treating”) refers to clinical intervention in an attempt to alter the natural course of a disease in the individual being treated, performed during the course of clinical pathology. Desirable effects of treatment include, but are not limited to, alleviation of symptoms, diminishment of any direct or indirect pathological consequences of the disease, decreasing the rate of disease progression, amelioration or palliation of the disease state, and remission or improved prognosis. For example, an individual is successfully “treated” if one or more symptoms associated with the disease are mitigated or eliminated, the individual's quality of life is improved, the dose of other medications required to treat the disease can be reduced, and/or the individual's survival is prolonged.

An “effective amount” of an agent, e.g., a pharmaceutical composition, refers to an amount of the antibody or medicament effective, at dosages and for periods of time necessary, to achieve the desired treatment as defined above.

An “individual” or “subject” is a mammal. In one aspect, the individual or subject is a human. In one aspect, the individual is in need of treatment.

The term “package insert” is used to refer to instructions customarily included in commercial packages of therapeutic products, that contain information about the indications, usage, dosage, administration, combination therapy, contraindications and/or warnings concerning the use of such therapeutic products.

### Methods and Compositions

### Therapeutic Methods

In one aspect, the present invention provides a method of treating an inflammatory lung disease in an individual, comprising administering to the individual a combination of a) an IL-33 inhibitor and b) an IL-6 inhibitor.

The invention further provides an IL-33 inhibitor for use the treatment of an inflammatory lung disease in an individual, wherein said treatment comprises administering to the individual a combination of a) the IL-33 inhibitor and b) an IL-6 inhibitor. The invention also provides an IL-33 inhibitor for use in method of treating an inflammatory lung disease in an individual, comprising administering to the individual a combination of a) the IL-33 inhibitor and b) an IL-6 inhibitor.

Still further, the invention provides an IL-6 inhibitor for use in the treatment of an inflammatory lung disease in an individual, wherein said treatment comprises administering to the individual a combination of a) an IL-33 inhibitor and b) the IL-6 inhibitor. The invention further provides an IL-6 inhibitor for use in method of treating an inflammatory lung disease in an individual, comprising administering to the individual a combination of a) an IL-33 inhibitor and b) the IL-6 inhibitor.

The invention also provides the use of an IL-33 inhibitor in the manufacture of a medicament for the treatment of an inflammatory lung disease in an individual, wherein said treatment comprises administering to the individual a combination of a) the IL-33 inhibitor and b) an IL-6 inhibitor. The invention further provides the use of an IL-33 inhibitor in the manufacture of a medicament for use in method of treating an inflammatory lung disease in an individual, comprising administering to the individual a combination of a) the IL-33 inhibitor and b) an IL-6 inhibitor.

Further, the invention provides the use of an IL-6 inhibitor in the manufacture of a medicament for the treatment of an inflammatory lung disease in an individual, wherein said treatment comprises administering to the individual a combination of a) an IL-33 inhibitor and b) the IL-6 inhibitor. The invention further provides the use of an IL-6 inhibitor in the manufacture of a medicament for use in method of treating an inflammatory lung disease in an individual, comprising administering to the individual a combination of a) an IL-33 inhibitor and b) the IL-6 inhibitor.

In one aspect, the invention also provides a kit comprising a first medicament comprising a IL-33 inhibitor and a second medicament comprising a IL-6 inhibitor, and optionally further comprising a package insert comprising instructions for administration of the first medicament in combination with the second medicament for treating an inflammatory lung disease in an individual.

According to the invention, in a preferred aspect, the inflammatory lung disease is chronic obstructive pulmonary disease (COPD). In another aspect, the inflammatory lung disease is asthma. In yet another aspect, the inflammatory lung disease is interstitial lung disease (ILD), in particular idiopathic pulmonary fibrosis (IPF). In still another aspect, the inflammatory lung disease is non-cystic fibrosis bronchiectasis (NCFB).

In one aspect, the administration of the combination of the IL-33 inhibitor and the IL-6 inhibitor results in enhanced therapeutic efficacy (in the treatment of the inflammatory lung disease) as compared to that observed with the administration of the IL-33 inhibitor or the IL-6 inhibitor alone.

As used herein, “combination” (and grammatical variations thereof such as “combine” or “combining”) encompasses combinations of an IL-33 inhibitor and an IL-6 inhibitor according to the invention wherein the IL-33 inhibitor and the IL-6 inhibitor are in the same or in different containers, in the same or in different pharmaceutical formulations, administered together or separately, administered simultaneously or sequentially, in any order, and administered by the same or by different routes, provided that the IL-33 inhibitor and the IL-6 inhibitor can simultaneously exert their biological effects in the body. For example “combining” an IL-33 inhibitor and an IL-6 inhibitor according to the invention may mean first administering the IL-33 inhibitor in a particular pharmaceutical formulation, followed by administration of the IL-6 inhibitor in another pharmaceutical formulation, or vice versa.

Combination therapies as provided herein encompass combined administration (where the IL-33 inhibitor and the IL-6 inhibitor are included in the same or separate pharmaceutical composition(s)), and separate administration, in which case administration of the IL-33 inhibitor can occur prior to, simultaneously, and/or following, administration of the IL-6 inhibitor. In one aspect, administration of the IL-33 inhibitor and administration of the IL-6 inhibitor occur within about one, two, three, four, five, or six days, within about one, two or three weeks, or within about one month, of each other. In one aspect, the IL-33 inhibitor and the IL-6 inhibitor are administered to the individual on the same day, e.g. on the first day of the treatment. In one aspect, the IL-33 inhibitor and the IL-6 inhibitor are administered to the individual on two consecutive days, e.g. on the first and the second day of the treatment.

The IL-33 inhibitor and the IL-6 inhibitor may be administered in any suitable manner known in the art. They are administered in effective amounts. In one aspect, the IL-33 inhibitor and the IL-6 inhibitor are administered sequentially (at different times). In another aspect, the IL-33 inhibitor and the IL-6 inhibitor are administered concurrently (at the same time). In some aspects, the IL-33 inhibitor is in a separate composition as the IL-6 inhibitor. In some aspects, the IL-33 inhibitor is in the same composition as the IL-6 inhibitor.

The IL-33 inhibitor and the IL-6 inhibitor may, in preferred aspects, also be comprised in the same molecule, as described herein.

The IL-6 inhibitor and the IL-33 inhibitor that may be used in the present invention are further described in the following.

In one aspect, the IL-6 inhibitor is an antibody. In one aspect, the IL-6 inhibitor is a blocking antibody. In one aspect, the IL-6 inhibitor inhibits the interaction of IL-6 with IL-6R. In one aspect, the IL-6 inhibitor is an anti-IL-6 antibody or an anti-IL-6R antibody. In one aspect, the IL-6 inhibitor is an anti-IL-6 antibody. In one aspect, such anti-IL-6 antibody is vamikibart (WHO Drug Information, vol. 37, no. 3, 2023 (Recommended INN List 90), p. 881). In one aspect, such anti-IL-6 antibody is olokizumab (WHO Drug Information, vol. 25, no. 1, 2011 (Recommended INN List 65), p. 77). In one aspect, such anti-IL-6 antibody is satralizumab (WHO Drug Information, vol. 31, no. 3, 2017 (Recommended INN List 78), p. 552). In one aspect, such anti-IL-6 antibody is sirukumab (WHO Drug Information, vol. 26, No. 1, 2012 (Recommended INN List 67), p. 86). In one aspect, such anti-IL-6 antibody is clazakizumab (WHO Drug Information, vol. 27, No. 1, 2013 (Recommended INN List 69), p. 50). In one aspect, such anti-IL-6 antibody is siltuximab (WHO Drug Information, vol. 23, No. 3, 2009 (Recommended INN List 62), p. 262). Other anti-IL-6 antibodies may also be useful. In another aspect, the IL-6 inhibitor is an anti-IL-6R antibody. In one aspect, such anti-IL-6R antibody is tocilizumab (WHO Drug Information, vol. 18, no. 3, 2004 (Recommended INN List 52), p. 262). In one aspect, such anti-IL-6R antibody is sarilumab (WHO Drug Information, vol. 26, No. 3, 2012 (Recommended INN List 68), p. 321). Other anti-IL-6R antibodies may also be useful.

In one aspect, the IL-6 inhibitor is an anti-IL-6 antibody, comprising a heavy chain variable region (VH) comprising the heavy chain complementarity determining region (HCDR) 1 of SEQ ID NO: 1, the HCDR 2 of SEQ ID NO: 2 and the HCDR 3 of SEQ ID NO: 3, and a light chain variable region (VL) comprising the light chain complementarity determining region (LCDR) 1 of SEQ ID NO: 5, the LCDR 2 of SEQ ID NO: 6 and the LCDR 3 of SEQ ID NO: 7. In one aspect, the IL-6 inhibitor is an anti-IL-6 antibody, comprising a heavy chain variable region (VH) comprising an amino acid sequence having at least 95%, at least 96%, at least 97%, at least 98% or at least 99% sequence identity to the VH sequence of SEQ ID NO: 4, and a light chain variable region (VL) comprising an amino acid sequence having at least 95%, at least 96%, at least 97%, at least 98% or at least 99% sequence identity to the VL sequence of SEQ ID NO: 8. In one aspect, the IL-6 inhibitor is an anti-IL-6 antibody, comprising a heavy chain variable region (VH) comprising the amino acid sequence of SEQ ID NO: 4, and a light chain variable region (VL) comprising the amino acid sequence of SEQ ID NO: 8.

In one aspect, the IL-33 inhibitor is an antibody. In one aspect, the IL-33 inhibitor is a blocking antibody. In one aspect, the IL-33 inhibitor inhibits the interaction of IL-33 with ST2. In one aspect, the IL-33 inhibitor is an anti-IL-33 antibody or an anti-ST2 antibody. In one aspect, the IL-33 inhibitor is an anti-IL-33 antibody. In one aspect, such anti-IL-33 antibody is 1E1v8 as described in PCT publication no. WO 2021/183849 (see SEQ ID NOs 7 and 8 of WO 2021/183849 for the VH and VL sequences of 1E1v8, respectively). In one aspect, such anti-IL-33 antibody is tozorakimab (WHO Drug Information, vol. 35, no. 3, 2021 (Recommended INN List 86), p. 825). In one aspect, such anti-IL-33 antibody is itepekimab (WHO Drug Information, vol. 34, no. 3, 2020 (Recommended INN List 84), p. 731). In one aspect, such anti-IL-33 antibody is etokimab (WHO Drug Information, vol. 33, no. 3, 2019 (Recommended INN List 82), p. 636). In one aspect, such anti-IL-33 antibody is torudokimab (WHO Drug Information, vol. 35, no. 3, 2021 (Recommended INN List 86), p. 823). Other anti-IL-33 antibodies, such as PF-06817024 or MT-2990, may also be useful. In another aspect, the IL-33 inhibitor is an anti-ST2 antibody. In one aspect, such anti-ST2 antibody is astegolimab (WHO Drug Information, vol. 34, no. 1, 2020 (Recommended INN List 83), p. 7). Other anti-ST2 antibodies, such as GSK3772847, may also be useful.

In one aspect, the IL-33 inhibitor is an anti-IL-33 antibody, comprising a heavy chain variable region comprising the heavy chain complementarity determining region (HCDR) 1 of SEQ ID NO: 9, the HCDR 2 of SEQ ID NO: 10 and the HCDR 3 of SEQ ID NO: 11, and a light chain variable region (VL) comprising the light chain complementarity determining region (LCDR) 1 of SEQ ID NO: 13, the LCDR 2 of SEQ ID NO: 14 and the LCDR 3 of SEQ ID NO: 15. In one aspect, the IL-33 inhibitor is an anti-IL-33 antibody, comprising a heavy chain variable region comprising an amino acid sequence having at least 95%, at least 96%, at least 97%, at least 98% or at least 99% sequence identity to the VH sequence of SEQ ID NO: 12, and a light chain variable region (VL) comprising an amino acid sequence having at least 95%, at least 96%, at least 97%, at least 98% or at least 99% sequence identity to the VH sequence of SEQ ID NO: 16. In one aspect, the IL-33 inhibitor is an anti-IL-33 antibody, comprising a heavy chain variable region comprising the amino acid sequence of SEQ ID NO: 12, and a light chain variable region (VL) comprising the amino acid sequence of SEQ ID NO: 16.

In preferred aspects, the IL-33 inhibitor and the IL-6 inhibitor are comprised in the same molecule. In a particular aspect, the IL-33 inhibitor and the IL-6 inhibitor are each an antibody comprised in a multispecific, particularly bispecific, antibody.

Combining the IL-33 inhibitor and the IL-6 inhibitor in a single molecule, such as in a bispecific antibody, provides for improved convenience in therapeutic application in that it requires administration (e.g. intravenous infusion or subcutaneous injection) of only one molecule. It may also reduce cost of treatment.

The IL-33 inhibitor and the IL-6 inhibitor according to this aspect of the invention may be any of 30 the anti-IL-33 or anti-ST2 antibodies and the anti-IL-6 or anti-IL-6R antibodies described herein, respectively. However, without wishing to be bound by theory, if comprised in a multispecific antibody, it may be preferable for the IL-33 inhibitor and the IL-6 inhibitor to be anti-IL-33 and IL-6 antibodies, respectively. This will minimize degradation of the antibody (target-mediated drug disposition, TMDD), which may occur upon binding of an antibody to a cell-surface target such as IL-6R or ST2.

Accordingly, in one aspect, the invention provides a method of treating a disease, particularly an inflammatory lung disease, in an individual, comprising administering to the individual (an effective amount of) an anti-IL-33/anti-IL-6 bispecific antibody.

The invention also provides an anti-IL-33/anti-IL-6 bispecific antibody for use as a medicament. Further provided by the invention is an anti-IL-33/anti-IL-6 bispecific antibody for use in the treatment of a disease, particularly an inflammatory lung disease. The invention further provides an anti-IL-33/anti-IL-6 bispecific antibody for use in a method of treatment. Also provided by the invention is an anti-IL-33/anti-IL-6 bispecific antibody for use in a method of treating an individual having a disease, particularly an inflammatory lung disease, comprising administering (an effective amount of) the anti-IL-33/anti-IL-6 bispecific antibody to the individual.

The invention also provides the use of an anti-IL-33/anti-IL-6 bispecific antibody in the manufacture of a medicament. Further provided is the use of an anti-IL-33/anti-IL-6 bispecific antibody in the manufacture of a medicament for the treatment of a disease, particularly an inflammatory lung disease. Further provided is the use of an anti-IL-33/anti-IL-6 bispecific antibody in the manufacture of a medicament for use in a method of treating an individual having a disease, particularly an inflammatory lung disease, comprising administering (an effective amount of) the medicament to the individual.

The invention also provides a medicament comprising an anti-IL-33/anti-IL-6 bispecific antibody. In one aspect, the medicament is (adapted) for the treatment of a disease, particularly an inflammatory lung disease. In one aspect, the medicament is (adapted) for use in a method of treating an individual having a disease, particularly an inflammatory lung disease, comprising administering (an effective amount of) the medicament to the individual.

Again, in a preferred aspect according to any of the above aspects, the inflammatory lung disease is chronic obstructive pulmonary disease (COPD). In another aspect, the inflammatory lung disease is asthma. In yet another aspect, the inflammatory lung disease is interstitial lung disease (ILD), in particular idiopathic pulmonary fibrosis (IPF). In still another aspect, the inflammatory lung disease is non-cystic fibrosis bronchiectasis (NCFB).

An “individual” according to any of the above aspects is preferably a human. An individual according to any of the above aspects may be in need of the medicament and/or treatment as described herein.

The method according to any of the above aspects may or may not further comprise administering to the individual an additional therapeutic agent. In one aspect, the method further comprises administering to the individual (an effective amount of) at least one additional therapeutic agent, particularly a therapeutic agent indicated for the treatment of an inflammatory lung disease, in particular COPD. In another aspect, the method does not comprise administering to the individual (an effective amount of) an additional therapeutic agent, for example an additional antibody or an additional cytokine inhibitor (including an additional antibody that binds to a cytokine or to a cytokine receptor).

The therapeutic agents according to the invention (e.g., an IL-33 inhibitor, an IL-6 inhibitor, an anti-IL-33/anti-IL-6 bispecific antibody) can be administered by any suitable means, including parenteral, intrapulmonary, or intranasal administration. Parenteral administration includes intramuscular, intravenous, intraarterial, intraperitoneal, or subcutaneous administration. In one aspect, administration of a therapeutic agent according to the invention (e.g., an IL-33 inhibitor, an IL-6 inhibitor, an anti-IL-33/anti-IL-6 bispecific antibody) is intravenous. In another aspect, administration of a therapeutic agent according to the invention (e.g., an IL-33 inhibitor, an IL-6 inhibitor, an anti-IL-33/anti-IL-6 bispecific antibody) is subcutaneous. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The therapeutic agents according to the invention would be formulated, dosed, and administered in a fashion consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners. The therapeutic agents according to the invention need not be, but may optionally be formulated with one or more agents used to treat the disorder in question. The effective amount of such other agents depends on the amount of therapeutic agent present in the pharmaceutical composition, the type of disorder or treatment, and other factors discussed above.

For the treatment of disease, the appropriate dosage of a therapeutic agent according the invention will depend on the type of disease to be treated, the type of therapeutic agent, the severity and course of the disease, previous therapy, the patient's clinical history and response to the therapeutic agent, and the discretion of the attending physician. The therapeutic agent is suitably administered to the patient at one time or over a series of treatments. For repeated administrations over several days or longer, depending on the condition, the treatment would generally be sustained until a desired suppression of disease symptoms occurs. The progress of this therapy is easily monitored by conventional techniques and assays.

The anti-IL-33/anti-IL-6 bispecific antibody that may be used in the present invention is further described in the following, and also as such provided by the present invention.

### Anti-IL-33/Anti-IL-6 Bispecific Antibody

In one aspect, the present invention provides a bispecific antibody that binds IL-33 and IL-6 (also referred to herein as an “anti-IL-33/anti-IL-6 bispecific antibody”).

Techniques for making bispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., Carter et al., J Immunol Meth 248, 7-15 (2001)). Bispecific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)).

Engineered antibodies with three or more antigen binding sites, including for example, “Octopus antibodies”, or DVD-Ig are also included herein (see, e.g., WO 2001/77342 and WO 2008/024715). Other examples of bispecific antibodies with three or more antigen binding sites can be found in WO 2010/115589, WO 2010/112193, WO 2010/136172, WO 2010/145792, and WO 2013/026831. The bispecific antibody may also include a “Dual Acting FAb” or “DAF” comprising an antigen binding site that binds to two different antigens, or two different epitopes of the same antigen (see, e.g., US 2008/0069820 and WO 2015/095539). Bispecific antibodies also include a “DutaFab” wherein a single pair of a VH domain and a VL domain may bind to two different epitopes and wherein one paratope comprises amino acid residues from HCDR2, LCDR1 and LCDR3 and the other paratope comprises amino acid residues from HCDR1, HCDR3 and LCDR2 (see, e.g., WO 2012/163520).

Bispecific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299). Also see Schaefer et al, PNAS, 108 (2011) 1187-1191, and Klein at al., MAbs 8 (2016) 1010-20.

Asymmetrical Fab arms can also be engineered by introducing charged or non-charged amino acid mutations into domain interfaces to direct correct Fab (i.e. heavy and light chain) pairing. Exemplary Fab pairing amino acid mutations are Q39E (Kabat numbering) and S183K (Kabat EU numbering) in the heavy chain and Q38K (Kabat numbering) and V133E (Kabat EU numbering) in the light chain, or Q39K (Kabat numbering) and S183E (Kabat EU numbering) in the heavy chain and Q38E (Kabat numbering) and V133K (Kabat EU numbering) in the light chain (see e.g. WO 2016/172485). Further Fab pairing mutations include 124K, 124R or 124H (Kabat numbering) and 123K, 123R or 123H (Kabat numbering) in the light chain and 147E or 147D (Kabat EU numbering) and 213E or 213D (Kabat EU numbering) in the heavy chain (see e.g. WO 2015/150447).

To promote the correct association of heavy chains in asymmetric bispecific antibodies, their heavy chains may be engineered to comprise e.g. sterically (“knob-in-hole”) or electrostatically complementary amino acid mutations, salt bridges, and/or disulfide bonds. The knob-in-hole technology is described e.g. in U.S. Pat. Nos. 5,731,168; 7,695,936; Ridgway et al., Prot Eng 9, 617-621 (1996), Atwell et al., J. Mol. Biol. 270, 26 (1997), Merchant et al., Nat Biotechnol 16, 677-681 (1998), and Carter, J Immunol Meth 248, 7-15 (2001). According to the knob-in-hole technology, a bispecific antibody comprising a human IgG1 Fc domain may comprise the“knob” mutation T366W on the first heavy chain, and “hole” mutations Y407V and optionally T366S and L368A (all Kabat EU numbering) on the second heavy chain (T366W/T366S:L368A:Y407V). Additionally, the antibody may comprise a S354C substitution on the first heavy chain and a Y349C substitution (both Kabat EU numbering) on the second heavy chain, forming a disulfide bond ((T366W:S354C/Y349C:T366S:L368A:Y407V).

Further examples of amino acid mutations (e.g. substitutions) that may be comprised in multispecific (e.g. bispecific) antibodies include the substitution S228P (Kabat EU numbering) in antibodies comprising an IgG4 Fc domain, e.g. for preventing Fab arm exchange (see Stubenrauch et al., Drug Metabolism and Disposition 38, 84-91 (2010)).

Various further molecular formats for multispecific antibodies are known in the art and are included herein (see e.g., Spiess et al., Mol Immunol 67: 95-106 (2015)).

Details of the anti-IL-33/anti-IL-6 bispecific antibody of the present invention are further described in the following.

In one aspect, the anti-IL-33/anti-IL-6 bispecific antibody comprises
(a) a first antigen binding domain that binds to IL-6, and
(b) a second antigen binding domain that binds to IL-33.

According to preferred aspects of the invention, the antigen binding domains comprised in the bispecific antibody are Fab molecules (i.e. antigen binding domains composed of a heavy and a light chain, each comprising a variable and a constant domain).

In a preferred aspect, one of the antigen binding domains is a crossover Fab molecule and the other one is a conventional Fab molecule. Such configuration reduces mispairing of heavy and light chains from different Fab molecules, thereby improving the yield and purity of the bispecific antibody in recombinant production. In a preferred crossover Fab molecule useful for the bispecific antibody of the invention, the variable domains of the Fab light chain and the Fab heavy chain (VL and VH, respectively) are exchanged. Even with this domain exchange, however, the preparation of the bispecific antibody may comprise certain side products due to a so-called Bence Jones-type interaction between mispaired heavy and light chains (see Schaefer et al, PNAS, 108 (2011) 11187-11191). To further reduce mispairing of heavy and light chains from different Fab molecules and thus increase the purity and yield of the desired bispecific antibody, charged amino acids with opposite charges may be introduced at specific amino acid positions in the CH1 and CL domains of either of the Fab molecules (but not in both), as further described herein. Preferably, the charge modifications are made in the conventional Fab molecule comprised in the bispecific antibody.

In one aspect, the bispecific antibody comprises an Fc domain, particularly an IgG Fc domain, more particularly an IgG₁ Fc domain. In one aspect the Fc domain is a human Fc domain. In one aspect, the Fc domain is a human IgG₁ Fc domain. The Fc domain is composed of a first and a second subunit and may incorporate any of the features, singly or in combination, described in hereinbelow in relation to Fc domains, including Fc domain variants.

In one aspect, the bispecific antibody is an IgG, particularly an IgG₁, antibody. In one aspect, the antibody is a full-length antibody, e.g. a full-length IgG, particularly IgG₁, antibody or other antibody class or isotype as defined herein.

In one aspect, the bispecific antibody is an isolated antibody and/or a purified antibody.

In one aspect, the bispecific antibody is a monoclonal antibody.

In a further aspect, the bispecific antibody according to any of the above aspects may incorporate any of the features, singly or in combination, as described in sections 1.-5. below.

### 1. First Antigen Binding Domain

The first antigen binding domain of the anti-IL-33/anti-IL-6 antibody binds to IL-6, specifically human IL-6.

Exemplary IL-6 binding domains that may be used in the bispecific antibody include the antigen binding domains of vamikibart (WHO Drug Information, vol. 37, no. 3, 2023 (Recommended INN List 90), p. 881), olokizumab (WHO Drug Information, vol. 25, no. 1, 2011 (Recommended INN List 65), p. 77), satralizumab (WHO Drug Information, vol. 31, no. 3, 2017 (Recommended INN List 78), p. 552), sirukumab (WHO Drug Information, vol. 26, No. 1, 2012 (Recommended INN List 67), p. 86), clazakizumab (WHO Drug Information, vol. 27, No. 1, 2013 (Recommended INN List 69), p. 50) or siltuximab (WHO Drug Information, vol. 23, No. 3, 2009 (Recommended INN List 62), p. 262). Antigen binding domains of other anti-IL-6 antibodies may also be useful. In one 20 aspect, the IL-6 binding domain of the anti-IL-33/anti-IL-6 antibody has a K_{D} of ≤1 pM, particularly a K_{D} of ≤0.5 pM, as measured by SPR at 25° C. In a particular aspect, the IL-6 binding domain of the anti-IL-33/anti-IL-6 antibody is the antigen binding domain of vamikibart.

In particular aspects, the first antigen binding domain comprises a heavy chain variable region (VH_{IL-6}) and a light chain variable region (VL_{IL-6}).

In one aspect, the VH_{IL-6} comprises the heavy chain complementarity determining region (HCDR) 1 of SEQ ID NO: 1, the HCDR 2 of SEQ ID NO: 2, and the HCDR 3 of SEQ ID NO: 3.

In one aspect, the first antigen binding domain is a humanized antigen binding domain (i.e. an antigen binding domain derived from a humanized antibody). In one aspect, VH_{IL-6} is a humanized variable region. In one aspect, VH_{IL-6} comprises an acceptor human framework, e.g. a human immunoglobulin framework or a human consensus framework.

In some aspects, the VH_{IL-6} comprises one or more heavy chain framework sequence (i.e. the FR1, FR2, FR3 and/or FR4 sequence) of the heavy chain variable region sequence of SEQ ID NO: 4.

In one aspect, the VH_{IL-6} comprises an amino acid sequence having at least 95%, at least 96%, at least 97%, at least 98% or at least 99% sequence identity to the VH sequence of SEQ ID NO: 4. In one aspect, the VH_{IL-6} comprises an amino acid sequence having at least 95% sequence identity to the VH sequence of SEQ ID NO: 4. In one aspect, the VH_{IL-6} comprises an amino acid sequence having at least 98% sequence identity to the VH sequence of SEQ ID NO: 4.

In one aspect, a VH sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to IL-6. In one aspect, a total of 1 to 10 amino acids have been substituted, inserted and/or deleted in the amino acid sequence of SEQ ID NO: 4. In one aspect, substitutions, insertions, or deletions occur in regions outside the CDRs (i.e., in the FRs).

In one aspect, the VH_{IL-6} comprises the amino acid sequence of SEQ ID NO: 4 comprising up to two (i.e. 0, 1 or 2) amino acid substitutions within said amino acid sequence. In one aspect, the VH_{IL-6} comprises the amino acid sequence of SEQ ID NO: 4 comprising one amino acid substitution within said amino acid sequence. In one aspect, the VH_{IL-6} comprises the amino acid sequence of SEQ ID NO: 4 comprising two amino acid substitutions within said amino acid sequence. In one aspect, the substitutions are in the FR of the VH.

In one aspect, the VH_{IL-6} comprises the HCDR 1 of SEQ ID NO: 1, the HCDR 2 of SEQ ID NO: 2, and the HCDR 3 of SEQ ID NO: 3, and an amino acid sequence having at least 95%, at least 96%, at least 97%, at least 98% or at least 99% sequence identity to the amino acid sequence of SEQ ID NO: 4. In one aspect, the VH_{IL-6} comprises the HCDR 1 of SEQ ID NO: 1, the HCDR 2 of SEQ ID NO: 2, and the HCDR 3 of SEQ ID NO: 3, and an amino acid sequence having at least 95% sequence identity to the amino acid sequence of SEQ ID NO: 4. In one aspect, the VH_{IL-6} comprises the HCDR 1 of SEQ ID NO: 1, the HCDR 2 of SEQ ID NO: 2, and the HCDR 3 of SEQ ID NO: 3, and an amino acid sequence having at least 98% sequence identity to the amino acid sequence of SEQ ID NO: 4.

In one aspect, the VH_{IL-6} comprises the HCDR 1 of SEQ ID NO: 1, the HCDR 2 of SEQ ID NO: 2, and the HCDR 3 of SEQ ID NO: 3, and the amino acid sequence of SEQ ID NO: 4 comprising up to two (i.e. 0, 1 or 2) amino acid substitutions within (the framework region of) said amino acid sequence. In one aspect, the VH_{IL-6} comprises the HCDR 1 of SEQ ID NO: 1, the HCDR 2 of SEQ ID NO: 2, and the HCDR 3 of SEQ ID NO: 3, and the amino acid sequence of SEQ ID NO: 4 comprising one amino acid substitution within (the framework region of) said amino acid sequence. In one aspect, the VH_{IL-6} comprises the HCDR 1 of SEQ ID NO: 1, the HCDR 2 of SEQ ID NO: 2, and the HCDR 3 of SEQ ID NO: 3, and the amino acid sequence of SEQ ID NO: 4 comprising two amino acid substitutions within (the framework region of) said amino acid sequence.

In one aspect, the VH_{IL-6} comprises the heavy chain CDR sequences (i.e. HCDR1, HCDR2 and HCDR3 amino acid sequences) of the VH of SEQ ID NO: 4.

In one aspect, the VH_{IL-6} comprises the heavy chain CDR sequences of the VH of SEQ ID NO: 4, and a framework sequence having at least 95%, at least 96%, at least 97%, at least 98% or at least 99% sequence identity to the framework sequence of the VH of SEQ ID NO: 4. In one aspect, the VH_{IL-6} comprises the heavy chain CDR sequences of the VH of SEQ ID NO: 4, and a framework sequence having at least 95% sequence identity to the framework sequence of the VH of SEQ ID NO: 4. In one aspect, the VH_{IL-6} comprises the heavy chain CDR sequences of the VH of SEQ ID NO: 4, and a framework sequence having at least 98% sequence identity to the framework sequence of the VH of SEQ ID NO: 4.

In one aspect, the VH_{IL-6} comprises the heavy chain CDR sequences of the VH of SEQ ID NO: 4, and the framework sequence of the VH of SEQ ID NO: 4 comprising up to two (i.e. 0, 1 or 2) amino acid substitutions within said framework sequence. In one aspect, the VH_{IL-6} comprises the heavy chain CDR sequences of the VH of SEQ ID NO: 4, and the framework sequence of the VH of SEQ ID NO: 4 comprising one amino acid substitution within said framework sequence. In one aspect, the VH_{IL-6} comprises the heavy chain CDR sequences of the VH of SEQ ID NO: 4, and the framework sequence of the VH of SEQ ID NO: 4 comprising two amino acid substitutions within said framework sequence.

Preferably, the CDR sequences of the VH according to the above aspects are according to the Kabat definition. Alternatively, the CDRs of the VH according to the above aspects are according to the Chothia definition. Further alternatively, the CDRs of the VH according to the above aspects are according to the Contact definition. Still further alternatively, the CDRs of the VH according to the above aspects are according to the IMGT definition.

In one aspect, the VH_{IL-6} comprises the amino acid sequence of SEQ ID NO: 4. Optionally, the VH_{IL-6} comprises the amino acid sequence of SEQ ID NO: 4, including post-translational modifications of that sequence.

In one aspect, the VH_{IL-6} comprises the heavy chain CDR sequences of vamikibart. In one aspect, the VH_{IL-6} comprises the VH sequence of vamikibart.

In one aspect, the VL_{IL-6} comprises the light chain complementarity determining region (LCDR) 1 of SEQ ID NO: 5, the LCDR 2 of SEQ ID NO: 6, and the LCDR 3 of SEQ ID NO: 7.

In one aspect, the first antigen binding domain is a humanized antigen binding domain (i.e. an antigen binding domain derived from a humanized antibody). In one aspect, VL_{IL-6} is a humanized variable region. In one aspect, VL_{IL-6} comprises an acceptor human framework, e.g. a human immunoglobulin framework or a human consensus framework.

In some aspects, the VL_{IL-6} comprises one or more heavy chain framework sequence (i.e. the FR1, FR2, FR3 and/or FR4 sequence) of the heavy chain variable region sequence of SEQ ID NO: 8.

In one aspect, the VL_{IL-6} comprises an amino acid sequence having at least 95%, at least 96%, at least 97%, at least 98% or at least 99% sequence identity to the VL sequence of SEQ ID NO: 8. In one aspect, the VL_{IL-6} comprises an amino acid sequence having at least 95% sequence identity to the VL sequence of SEQ ID NO: 8. In one aspect, the VL_{IL-6} comprises an amino acid sequence having at least 98% sequence identity to the VL sequence of SEQ ID NO: 8.

In one aspect, a VL sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to IL-6. In one aspect, a total of 1 to 10 amino acids have been substituted, inserted and/or deleted in the amino acid sequence of SEQ ID NO: 8. In one aspect, substitutions, insertions, or deletions occur in regions outside the CDRs (i.e., in the FRs).

In one aspect, the VL_{IL-6} comprises the amino acid sequence of SEQ ID NO: 8 comprising up to two (i.e. 0, 1 or 2) amino acid substitutions within said amino acid sequence. In one aspect, the VL_{IL-6} comprises the amino acid sequence of SEQ ID NO: 8 comprising one amino acid substitution within said amino acid sequence. In one aspect, the VL_{IL-6} comprises the amino acid sequence of SEQ ID NO: 8 comprising two amino acid substitutions within said amino acid sequence. In one aspect, the substitutions are in the FR of the VL.

In one aspect, the VL_{IL-6} comprises the LCDR 1 of SEQ ID NO: 5, the LCDR 2 of SEQ ID NO: 6, and the LCDR 3 of SEQ ID NO: 7, and an amino acid sequence having at least 95%, at least 96%, at least 97%, at least 98% or at least 99% sequence identity to the amino acid sequence of SEQ ID NO: 8. In one aspect, the VL_{IL-6} comprises the LCDR 1 of SEQ ID NO: 5, the LCDR 2 of SEQ ID NO: 6, and the LCDR 3 of SEQ ID NO: 7, and an amino acid sequence having at least 95% sequence identity to the amino acid sequence of SEQ ID NO: 8. In one aspect, the VL_{IL-6} comprises the LCDR 1 of SEQ ID NO: 5, the LCDR 2 of SEQ ID NO: 6, and the LCDR 3 of SEQ ID NO: 7, and an amino acid sequence having at least 98% sequence identity to the amino acid sequence of SEQ ID NO: 8.

In one aspect, the VL_{IL-6} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising up to two (i.e. 0, 1 or 2) amino acid substitutions within (the framework region of) said amino acid sequence. In one aspect, the VL_{IL-6} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising one amino acid substitution within (the framework region of) said amino acid sequence. In one aspect, the VL_{IL-6} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising two amino acid substitutions within (the framework region of) said amino acid sequence.

In one aspect, the VL_{IL-6} comprises the light chain CDR sequences (i.e. LCDR1, LCDR2 and LCDR3 amino acid sequences) of the VL of SEQ ID NO: 8.

In one aspect, the VL_{IL-6} comprises the light chain CDR sequences of the VL of SEQ ID NO: 8, and a framework sequence having at least 95%, at least 96%, at least 97%, at least 98% or at least 99% sequence identity to the framework sequence of the VL of SEQ ID NO: 8. In one aspect, the VL_{IL-6} comprises the light chain CDR sequences of the VL of SEQ ID NO: 8, and a framework sequence having at least 95% sequence identity to the framework sequence of the VL of SEQ ID NO: 8. In one aspect, the VL_{IL-6} comprises the light chain CDR sequences of the VL of SEQ ID NO: 8, and a framework sequence having at least 98% sequence identity to the framework sequence of the VL of SEQ ID NO: 8.

In one aspect, the VL_{IL-6} comprises the light chain CDR sequences of the VL of SEQ ID NO: 8, and the framework sequence of the VH of SEQ ID NO: 8 comprising up to two (i.e. 0, 1 or 2) amino acid substitutions within said framework sequence. In one aspect, the VL_{IL-6} comprises the light chain CDR sequences of the VL of SEQ ID NO: 8, and the framework sequence of the VL of SEQ ID NO: 8 comprising one amino acid substitution within said framework sequence. In one aspect, the VL_{IL-6} comprises the light chain CDR sequences of the VL of SEQ ID NO: 8, and the framework sequence of the VL of SEQ ID NO: 8 comprising two amino acid substitutions within said framework sequence.

Preferably, the CDR sequences of the VL according to the above aspects are according to the Kabat definition. Alternatively, the CDRs of the VL according to the above aspects are according to the Chothia definition. Further alternatively, the CDRs of the VL according to the above aspects are according to the Contact definition. Still further alternatively, the CDRs of the VL according to the above aspects are according to the IMGT definition.

In one aspect, the VL_{IL-6} comprises the amino acid sequence of SEQ ID NO: 8. Optionally, the VL_{IL-6} comprises the amino acid sequence of SEQ ID NO: 8, including post-translational modifications of that sequence.

In one aspect, the VL_{IL-6} comprises the light chain CDR sequences of vamikibart. In one aspect, the VL_{IL-6} comprises the VL sequence of vamikibart.

In particular aspects, the first antigen binding domain comprises a VH sequence (VH_{IL-6}) as in any of the aspects provided above, and a VL sequence (VL_{IL-6}) as in any of the aspects provided above.

In one aspect, the first antigen binding domain comprises a heavy chain variable region (VH_{IL}-6) comprising the HCDR 1 of SEQ ID NO: 1, the HCDR 2 of SEQ ID NO: 2, and the HCDR 3 of SEQ ID NO: 3; and a light chain variable region (VL_{IL-6}) comprising the LCDR 1 of SEQ ID NO: 5, the LCDR 2 of SEQ ID NO: 6 and the LCDR 3 of SEQ ID NO: 7.

In one aspect, the first antigen binding domain comprises a heavy chain variable region (VH_{IL}-6) comprising an amino acid sequence having at least 95%, at least 96%, at least 97%, at least 98% or at least 99% sequence identity to the VH sequence of SEQ ID NO: 4; and a light chain variable region (VL_{IL-6}) comprising an amino acid sequence having at least 95%, at least 96%, at least 97%, at least 98% or at least 99% sequence identity to the VL sequence of SEQ ID NO: 8.

In one aspect, the first antigen binding domain comprises a heavy chain variable region (VH_{IL}-6) comprising the amino acid sequence of SEQ ID NO: 4 comprising up to two (i.e. 0, 1 or 2) amino acid substitutions within said amino acid sequence; and a light chain variable region (VL_{IL-6}) comprising the amino acid sequence of SEQ ID NO: 8 comprising up to two (i.e. 0, 1 or 2) amino acid substitutions within said amino acid sequence.

In one aspect, the first antigen binding domain comprises a heavy chain variable region (VH_{IL}-6) comprising the HCDR 1 of SEQ ID NO: 1, the HCDR 2 of SEQ ID NO: 2, and the HCDR 3 of SEQ ID NO: 3, and an amino acid sequence having at least 95%, at least 96%, at least 97%, at least 98% or at least 99% sequence identity to the amino acid sequence of SEQ ID NO: 4; and a light chain variable region (VL_{IL-6}) comprising the LCDR 1 of SEQ ID NO: 5, the LCDR 2 of SEQ ID NO: 6, and the LCDR 3 of SEQ ID NO: 7, and an amino acid sequence having at least 95%, at least 96%, at least 97%, at least 98% or at least 99% sequence identity to the amino acid sequence of SEQ ID NO: 8.

In one aspect, the first antigen binding domain comprises a heavy chain variable region (VH_{IL-6}) comprising the HCDR 1 of SEQ ID NO: 1, the HCDR 2 of SEQ ID NO: 2, and the HCDR 3 of SEQ ID NO: 3, and the amino acid sequence of SEQ ID NO: 4 comprising up to two (i.e. 0, 1 or 2) amino acid substitutions within (the framework region of) said amino acid sequence; and a light chain variable region (VL_{IL-6}) comprising the LCDR 1 of SEQ ID NO: 5, the LCDR 2 of SEQ ID NO: 6, and the LCDR 3 of SEQ ID NO: 7, and the amino acid sequence of SEQ ID NO: 8 comprising up to two (i.e. 0, 1 or 2) amino acid substitutions within (the framework region of) said amino acid sequence.

In one aspect, the first antigen binding domain comprises a heavy chain variable region (VH_{IL-6}) comprising the heavy chain CDR sequences (i.e. HCDR1, HCDR2 and HCDR3 amino acid sequences) of the VH of SEQ ID NO: 4; and a light chain variable region (VL_{IL-6}) comprising the light chain CDR sequences (i.e. LCDR1, LCDR2 and LCDR3 amino acid sequences) of the VL of SEQ ID NO: 8.

In one aspect, the first antigen binding domain comprises a heavy chain variable region (VH_{IL-6}) comprising the heavy chain CDR sequences of the VH of SEQ ID NO: 4, and a framework sequence having at least 95%, at least 96%, at least 97%, at least 98% or at least 99% sequence identity to the framework sequence of the VH of SEQ ID NO: 4; and a light chain variable region (VL_{IL-6}) comprising the light chain CDR sequences of the VL of SEQ ID NO: 8, and a framework sequence having at least 95%, at least 96%, at least 97%, at least 98% or at least 99% sequence identity to the framework sequence of the VL of SEQ ID NO: 8.

In one aspect, the first antigen binding domain comprises a heavy chain variable region (VH_{IL-6}) comprising the heavy chain CDR sequences of the VH of SEQ ID NO: 4, and the framework sequence of the VH of SEQ ID NO: 4 comprising up to two (i.e. 0, 1 or 2) amino acid substitutions within said framework sequence; and a light chain variable region (VL_{IL}-6) comprising the light chain CDR sequences of the VL of SEQ ID NO: 8, and the framework sequence of the VL of SEQ ID NO: 8 comprising up to two (i.e. 0, 1 or 2) amino acid substitutions within said framework sequence.

Preferably, the CDR sequences of the VH and/or VL according to the above aspects are according to the Kabat definition. Alternatively, the CDRs of the VH and/or VL according to the above aspects are according to the Chothia definition. Further alternatively, the CDRs of the VH and/or VL according to the above aspects are according to the Contact definition. Still further alternatively, the CDRs of the VH and/or VL according to the above aspects are according to the IMGT definition.

In one aspect, the first antigen binding domain comprises a heavy chain variable region (VH_{IL-6}) comprising the amino acid sequence of SEQ ID NO. 4; and a light chain variable region (VL_{IL-6}) comprising the amino acid sequence of SEQ ID NO. 8.

In one aspect, the first antigen binding domain comprises a heavy chain variable region (VH_{IL-6}) comprising the heavy chain CDR sequences of vamikibart, and a light chain variable region (VL_{IL-6}) comprising the light chain CDR sequences of vamikibart. In one aspect, the first antigen binding domain comprises a heavy chain variable region (VH_{IL-6}) comprising the VH sequence of vamikibart, and a light chain variable region (VL_{IL-6}) comprising the VL sequence of vamikibart.

In one aspect, the bispecific antibody comprises not more than one antigen binding domain that binds to IL-6. Accordingly, in such aspect, the bispecific antibody provides monovalent binding to IL-6.

In one aspect, the first antigen binding domain is an antibody fragment selected from the group of an Fv molecule, a scFv molecule and a Fab molecule. In a particular aspect, the first antigen binding domain is a Fab molecule.

In one aspect, the first antigen binding domain is a crossover Fab molecule as described herein, i.e. a Fab molecule wherein the variable domains VH and VL or the constant domains CH1 and CL of the Fab heavy and light chains are exchanged/replaced by each other. In such aspect, the second antigen binding domain is preferably a conventional Fab molecule.

In a preferred aspect, the first antigen binding domain is a Fab molecule wherein the variable domains VL and VH or the constant domains CL and CH1, particularly the variable domains VL and VH, of the Fab light chain and the Fab heavy chain are replaced by each other (i.e. according to such aspect, the first antigen binding domain is a crossover Fab molecule wherein the variable or constant domains of the Fab light chain and the Fab heavy chain are exchanged), and the second antigen binding domain is a conventional Fab molecule.

In one aspect, the first antigen binding domain comprises a human constant region. In one aspect, the first antigen binding domain is a Fab molecule comprising a human constant region, particularly a human CH1 and/or CL domain. In one aspect, the first antigen binding domain comprises a light chain constant region comprising an amino acid sequence that is at least 95%, at least 96%, at least 97%, at least 98%, at least 99% or 100% identical to the amino acid sequence of SEQ ID NO: 40. Particularly, in such aspect, the light chain constant region may comprise deletion or substitutions of one or more (particularly two)N-terminal amino acids if in a crossover Fab molecule. In some aspects, the first antigen binding domain comprises a heavy chain constant region comprising an amino acid sequence that is at least 95%, at least 96%, at least 97%, at least 98%, at least 99% or 100% identical to the CH1 domain sequence comprised in the amino acid sequence of SEQ ID NO: 42.

### 2. Second Antigen Binding Domain

The second antigen binding domain of the anti-IL-33/anti-IL-6 antibody binds to IL-33, specifically human IL-33.

Exemplary IL-33 binding domains that may be used in the bispecific antibody include the antigen binding domains of antibody 1E1v8 as described in PCT publication no. WO 2021/183849 (see SEQ ID NOs 7 and 8 of WO 2021/183849 for the VH and VL sequences of 1E1v8, respectively), tozorakimab (WHO Drug Information, vol. 35, no. 3, 2021 (Recommended INN List 86), p. 825), itepekimab (WHO Drug Information, vol. 34, no. 3, 2020 (Recommended INN List 84), p. 731), etokimab (WHO Drug Information, vol. 33, no. 3, 2019 (Recommended INN List 82), p. 636) or torudokimab (WHO Drug Information, vol. 35, no. 3, 2021 (Recommended INN List 86), p. 823).

Antigen binding domains of other anti-IL-33 antibodies, such as PF-06817024 or MT-2990, may 20 also be useful. In one aspect, the IL-33 binding domain of the anti-IL-33/anti-IL-6 antibody has a K_{D} of ≤1 pM, particularly a K_{D} of <0.5 pM, as measured by SPR at 25° C. In a particular aspect, the IL-33 binding domain of the anti-IL-33/anti-IL-6 antibody is the antigen binding domain of 1E1v8.

In particular aspects, the first antigen binding domain comprises a heavy chain variable region (VH_{IL-33}) and a light chain variable region (VL_{IL-33}).

In one aspect, the VH_{IL-33} comprises the heavy chain complementarity determining region (HCDR) 1 of SEQ ID NO: 9, the HCDR 2 of SEQ ID NO: 10, and the HCDR 3 of SEQ ID NO: 11.

In one aspect, the first antigen binding domain is a humanized antigen binding domain (i.e. an antigen binding domain derived from a humanized antibody). In one aspect, VH_{IL-33} is a humanized variable region. In one aspect, VH_{IL-33} comprises an acceptor human framework, e.g. a human immunoglobulin framework or a human consensus framework.

In some aspects, the VH_{IL-33} comprises one or more heavy chain framework sequence (i.e. the FR1, FR2, FR3 and/or FR4 sequence) of the heavy chain variable region sequence of SEQ ID NO: 12.

In one aspect, the VH_{IL-33} comprises an amino acid sequence having at least 95%, at least 96%, at least 97%, at least 98% or at least 99% sequence identity to the VH sequence of SEQ ID NO: 12. In one aspect, the VH_{IL-6} comprises an amino acid sequence having at least 95% sequence identity to the VH sequence of SEQ ID NO: 12. In one aspect, the VH_{IL-33} comprises an amino acid sequence having at least 98% sequence identity to the VH sequence of SEQ ID NO: 12.

In one aspect, a VH sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to IL-33. In one aspect, a total of 1 to 10 amino acids have been substituted, inserted and/or deleted in the amino acid sequence of SEQ ID NO: 12. In one aspect, substitutions, insertions, or deletions occur in regions outside the CDRs (i.e., in the FRs).

In one aspect, the VH_{IL-33} comprises the amino acid sequence of SEQ ID NO: 12 comprising up to two (i.e. 0, 1 or 2) amino acid substitutions within said amino acid sequence. In one aspect, the VH_{IL-6} comprises the amino acid sequence of SEQ ID NO: 12 comprising one amino acid substitution within said amino acid sequence. In one aspect, the VH_{IL-33} comprises the amino acid sequence of SEQ ID NO: 12 comprising two amino acid substitutions within said amino acid sequence. In one aspect, the substitutions are in the FR of the VH.

In one aspect, the VH_{IL-33} comprises the HCDR 1 of SEQ ID NO: 9, the HCDR 2 of SEQ ID NO: 10, and the HCDR 3 of SEQ ID NO: 11, and an amino acid sequence having at least 95%, at least 96%, at least 97%, at least 98% or at least 99% sequence identity to the amino acid sequence of SEQ ID NO: 12. In one aspect, the VH_{IL-33} comprises the HCDR 1 of SEQ ID NO: 9, the HCDR 2 of SEQ ID NO: 10, and the HCDR 3 of SEQ ID NO: 11, and an amino acid sequence having at least 95% sequence identity to the amino acid sequence of SEQ ID NO: 12. In one aspect, the VH_{IL-33} comprises the HCDR 1 of SEQ ID NO: 9, the HCDR 2 of SEQ ID NO: 10, and the HCDR 3 of SEQ ID NO: 11, and an amino acid sequence having at least 98% sequence identity to the amino acid sequence of SEQ ID NO: 12.

In one aspect, the VH_{IL-33} comprises the HCDR 1 of SEQ ID NO: 9, the HCDR 2 of SEQ ID NO: 10, and the HCDR 3 of SEQ ID NO: 11, and the amino acid sequence of SEQ ID NO: 12 comprising up to two (i.e. 0, 1 or 2) amino acid substitutions within (the framework region of) said amino acid sequence. In one aspect, the VH_{IL-33} comprises the HCDR 1 of SEQ ID NO: 9, the HCDR 2 of SEQ ID NO: 10, and the HCDR 3 of SEQ ID NO: 11, and the amino acid sequence of SEQ ID NO: 12 comprising one amino acid substitution within (the framework region of) said amino acid sequence. In one aspect, the VH_{IL-33} comprises the HCDR 1 of SEQ ID NO: 9, the HCDR 2 of SEQ ID NO: 10, and the HCDR 3 of SEQ ID NO: 11, and the amino acid sequence of SEQ ID NO: 12 comprising two amino acid substitutions within (the framework region of) said amino acid sequence.

In one aspect, the VH_{IL-33} comprises the heavy chain CDR sequences (i.e. HCDR1, HCDR2 and HCDR3 amino acid sequences) of the VH of SEQ ID NO: 12.

In one aspect, the VH_{IL-33} comprises the heavy chain CDR sequences of the VH of SEQ ID NO: 12, and a framework sequence having at least 95%, at least 96%, at least 97%, at least 98% or at least 99% sequence identity to the framework sequence of the VH of SEQ ID NO: 12. In one aspect, the VH_{IL-33} comprises the heavy chain CDR sequences of the VH of SEQ ID NO: 12, and a framework sequence having at least 95% sequence identity to the framework sequence of the VH of SEQ ID NO: 12. In one aspect, the VH_{IL-33} comprises the heavy chain CDR sequences of the VH of SEQ ID NO: 12, and a framework sequence having at least 98% sequence identity to the framework sequence of the VH of SEQ ID NO: 12.

In one aspect, the VH_{IL-33} comprises the heavy chain CDR sequences of the VH of SEQ ID NO: 12, and the framework sequence of the VH of SEQ ID NO: 12 comprising up to two (i.e. 0, 1 or 2) amino acid substitutions within said framework sequence. In one aspect, the VH_{IL-33} comprises the heavy chain CDR sequences of the VH of SEQ ID NO: 12, and the framework sequence of the VH of SEQ ID NO: 12 comprising one amino acid substitution within said framework sequence. In one aspect, the VH_{IL-33} comprises the heavy chain CDR sequences of the VH of SEQ ID NO: 12, and the framework sequence of the VH of SEQ ID NO: 12 comprising two amino acid substitutions within said framework sequence.

Preferably, the CDR sequences of the VH according to the above aspects are according to the Kabat definition. Alternatively, the CDRs of the VH according to the above aspects are according to the Chothia definition. Further alternatively, the CDRs of the VH according to the above aspects are according to the Contact definition. Still further alternatively, the CDRs of the VH according to the above aspects are according to the IMGT definition.

In one aspect, the VH_{IL-33} comprises the amino acid sequence of SEQ ID NO: 12. Optionally, the VH_{IL-6} comprises the amino acid sequence of SEQ ID NO: 12, including post-translational modifications of that sequence.

In one aspect, the VH_{IL-33} comprises the heavy chain CDR sequences of 1E1v8. In one aspect, the VH_{IL-6} comprises the VH sequence of 1E1v8.

In one aspect, the VL_{IL-33} comprises the light chain complementarity determining region (LCDR) 1 of SEQ ID NO: 13, the LCDR 2 of SEQ ID NO: 14, and the LCDR 3 of SEQ ID NO: 15.

In one aspect, the first antigen binding domain is a humanized antigen binding domain (i.e. an antigen binding domain derived from a humanized antibody). In one aspect, VL_{IL-33} is a humanized variable region. In one aspect, VL_{IL-33} comprises an acceptor human framework, e.g. a human immunoglobulin framework or a human consensus framework.

In some aspects, the VL_{IL-33} comprises one or more heavy chain framework sequence (i.e. the FR1, FR2, FR3 and/or FR4 sequence) of the heavy chain variable region sequence of SEQ ID NO: 16.

In one aspect, the VL_{IL-33} comprises an amino acid sequence having at least 95%, at least 96%, at least 97%, at least 98% or at least 99% sequence identity to the VL sequence of SEQ ID NO: 16.

In one aspect, the VL_{IL-33} comprises an amino acid sequence having at least 95% sequence identity to the VL sequence of SEQ ID NO: 16. In one aspect, the VL_{IL-33} comprises an amino acid sequence having at least 98% sequence identity to the VL sequence of SEQ ID NO: 16.

In one aspect, a VL sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to IL-33. In one aspect, a total of 1 to 10 amino acids have been substituted, inserted and/or deleted in the amino acid sequence of SEQ ID NO: 16. In one aspect, substitutions, insertions, or deletions occur in regions outside the CDRs (i.e., in the FRs).

In one aspect, the VL_{IL-33} comprises the amino acid sequence of SEQ ID NO: 16 comprising up to two (i.e. 0, 1 or 2) amino acid substitutions within said amino acid sequence. In one aspect, the VL_{IL-33} comprises the amino acid sequence of SEQ ID NO: 16 comprising one amino acid substitution within said amino acid sequence. In one aspect, the VL_{IL-33} comprises the amino acid sequence of SEQ ID NO: 16 comprising two amino acid substitutions within said amino acid sequence. In one aspect, the substitutions are in the FR of the VL.

In one aspect, the VL_{IL-33} comprises the LCDR 1 of SEQ ID NO: 13, the LCDR 2 of SEQ ID NO: 14, and the LCDR 3 of SEQ ID NO: 15, and an amino acid sequence having at least 95%, at least 96%, at least 97%, at least 98% or at least 99% sequence identity to the amino acid sequence of SEQ ID NO: 16. In one aspect, the VL_{IL-33} comprises the LCDR 1 of SEQ ID NO: 13, the LCDR 2 of SEQ ID NO: 14, and the LCDR 3 of SEQ ID NO: 15, and an amino acid sequence having at least 95% sequence identity to the amino acid sequence of SEQ ID NO: 16. In one aspect, the VL_{IL-33} comprises the LCDR 1 of SEQ ID NO: 13, the LCDR 2 of SEQ ID NO: 14, and the LCDR 3 of SEQ ID NO: 15, and an amino acid sequence having at least 98% sequence identity to the amino acid sequence of SEQ ID NO: 16.

In one aspect, the VL_{IL-33} comprises the LCDR 1 of SEQ ID NO: 13, the LCDR 2 of SEQ ID NO: 14, and the LCDR 3 of SEQ ID NO: 15, and the amino acid sequence of SEQ ID NO: 16 comprising up to two (i.e. 0, 1 or 2) amino acid substitutions within (the framework region of) said amino acid sequence. In one aspect, the VL_{IL-33} comprises the LCDR 1 of SEQ ID NO: 13, the LCDR 2 of SEQ ID NO: 14, and the LCDR 3 of SEQ ID NO: 15, and the amino acid sequence of SEQ ID NO: 16 comprising one amino acid substitution within (the framework region of) said amino acid sequence. In one aspect, the VL_{IL-33} comprises the LCDR 1 of SEQ ID NO: 13, the LCDR 2 of SEQ ID NO: 14, and the LCDR 3 of SEQ ID NO: 15, and the amino acid sequence of SEQ ID NO: 16 comprising two amino acid substitutions within (the framework region of) said amino acid sequence.

In one aspect, the VL_{IL-33} comprises the light chain CDR sequences (i.e. LCDR1, LCDR2 and LCDR3 amino acid sequences) of the VL of SEQ ID NO: 16.

In one aspect, the VL_{IL-33} comprises the light chain CDR sequences of the VL of SEQ ID NO: 16, and a framework sequence having at least 95%, at least 96%, at least 97%, at least 98% or at least 99% sequence identity to the framework sequence of the VL of SEQ ID NO: 16. In one aspect, the VL_{IL-33} comprises the light chain CDR sequences of the VL of SEQ ID NO: 16, and a framework sequence having at least 95% sequence identity to the framework sequence of the VL of SEQ ID NO: 16. In one aspect, the VL_{IL-33} comprises the light chain CDR sequences of the VL of SEQ ID NO: 16, and a framework sequence having at least 98% sequence identity to the framework sequence of the VL of SEQ ID NO: 16.

In one aspect, the VL_{IL-33} comprises the light chain CDR sequences of the VL of SEQ ID NO: 16, and the framework sequence of the VH of SEQ ID NO: 16 comprising up to two (i.e. 0, 1 or 2) amino acid substitutions within said framework sequence. In one aspect, the VL_{IL-33} comprises the light chain CDR sequences of the VL of SEQ ID NO: 16, and the framework sequence of the VL of SEQ ID NO: 16 comprising one amino acid substitution within said framework sequence. In one aspect, the VL_{IL-33} comprises the light chain CDR sequences of the VL of SEQ ID NO: 16, and the framework sequence of the VL of SEQ ID NO: 16 comprising two amino acid substitutions within said framework sequence.

Preferably, the CDR sequences of the VL according to the above aspects are according to the Kabat definition. Alternatively, the CDRs of the VL according to the above aspects are according to the Chothia definition. Further alternatively, the CDRs of the VL according to the above aspects are according to the Contact definition. Still further alternatively, the CDRs of the VL according to the above aspects are according to the IMGT definition.

In one aspect, the VL_{IL-33} comprises the amino acid sequence of SEQ ID NO: 16. Optionally, the VL_{IL-33} comprises the amino acid sequence of SEQ ID NO: 16, including post-translational modifications of that sequence.

In one aspect, the VL_{IL-33} comprises the light chain CDR sequences of 1E1v8. In one aspect, the VL_{IL-6} comprises the VL sequence of 1E1v8.

In particular aspects, the second antigen binding domain comprises a VH sequence (VH_{IL-33}) as in any of the aspects provided above, and a VL sequence (VL_{IL-33}) as in any of the aspects provided above.

In one aspect, the second antigen binding domain comprises a heavy chain variable region (VH_{IL-33}) comprising the HCDR 1 of SEQ ID NO: 9, the HCDR 2 of SEQ ID NO: 10, and the HCDR 3 of SEQ ID NO: 11; and a light chain variable region (VL_{IL-33}) comprising the LCDR 1 of SEQ ID NO: 13, the LCDR 2 of SEQ ID NO: 14 and the LCDR 3 of SEQ ID NO: 15.

In one aspect, the second antigen binding domain comprises a heavy chain variable region (VH_{IL-33}) comprising an amino acid sequence having at least 95%, at least 96%, at least 97%, at least 98% or at least 99% sequence identity to the VH sequence of SEQ ID NO: 12; and a light chain variable region (VL_{IL-33}) comprising an amino acid sequence having at least 95%, at least 96%, at least 97%, at least 98% or at least 99% sequence identity to the VL sequence of SEQ ID NO: 16.

In one aspect, the second antigen binding domain comprises a heavy chain variable region (VH_{IL-33}) comprising the amino acid sequence of SEQ ID NO: 12 comprising up to two (i.e. 0, 1 or 2) amino acid substitutions within said amino acid sequence; and a light chain variable region (VL_{IL-33}) comprising the amino acid sequence of SEQ ID NO: 16 comprising up to two (i.e. 0, 1 or 2) amino acid substitutions within said amino acid sequence.

In one aspect, the second antigen binding domain comprises a heavy chain variable region (VH_{IL-33}) comprising the HCDR 1 of SEQ ID NO: 9, the HCDR 2 of SEQ ID NO: 10, and the HCDR 3 of SEQ ID NO: 11, and an amino acid sequence having at least 95%, at least 96%, at least 97%, at least 98% or at least 99% sequence identity to the amino acid sequence of SEQ ID NO: 12; and a light chain variable region (VL_{IL-33}) comprising the LCDR 1 of SEQ ID NO: 13, the LCDR 2 of SEQ ID NO: 14, and the LCDR 3 of SEQ ID NO: 15, and an amino acid sequence having at least 95%, at least 96%, at least 97%, at least 98% or at least 99% sequence identity to the amino acid sequence of SEQ ID NO: 16.

In one aspect, the second antigen binding domain comprises a heavy chain variable region (VH_{IL-33}) comprising the HCDR 1 of SEQ ID NO: 9, the HCDR 2 of SEQ ID NO: 10, and the HCDR 3 of SEQ ID NO: 11, and the amino acid sequence of SEQ ID NO: 12 comprising up to two (i.e. 0, 1 or 2) amino acid substitutions within (the framework region of) said amino acid sequence; and a light chain variable region (VL_{IL-33}) comprising the LCDR 1 of SEQ ID NO: 13, the LCDR 2 of SEQ ID NO: 14, and the LCDR 3 of SEQ ID NO: 15, and the amino acid sequence of SEQ ID NO: 16 comprising up to two (i.e. 0, 1 or 2) amino acid substitutions within (the framework region of) said amino acid sequence.

In one aspect, the second antigen binding domain comprises a heavy chain variable region (VH_{IL-33}) comprising the heavy chain CDR sequences (i.e. HCDR1, HCDR2 and HCDR3 amino acid sequences) of the VH of SEQ ID NO: 12; and a light chain variable region (VL_{IL-33}) comprising the light chain CDR sequences (i.e. LCDR1, LCDR2 and LCDR3 amino acid sequences) of the VL of SEQ ID NO: 16.

In one aspect, the second antigen binding domain comprises a heavy chain variable region (VH_{IL-33}) comprising the heavy chain CDR sequences of the VH of SEQ ID NO: 12, and a framework sequence having at least 95%, at least 96%, at least 97%, at least 98% or at least 99% sequence identity to the framework sequence of the VH of SEQ ID NO: 12; and a light chain variable region (VL_{IL-33}) comprising the light chain CDR sequences of the VL of SEQ ID NO: 16, and a framework sequence having at least 95%, at least 96%, at least 97%, at least 98% or at least 99% sequence identity to the framework sequence of the VL of SEQ ID NO: 16.

In one aspect, the second antigen binding domain comprises a heavy chain variable region (VH_{IL-33}) comprising the heavy chain CDR sequences of the VH of SEQ ID NO: 12, and the framework sequence of the VH of SEQ ID NO: 12 comprising up to two (i.e. 0, 1 or 2) amino acid substitutions within said framework sequence; and a light chain variable region (VL_{IL-33}) comprising the light chain CDR sequences of the VL of SEQ ID NO: 16, and the framework sequence of the VL of SEQ ID NO: 16 comprising up to two (i.e. 0, 1 or 2) amino acid substitutions within said framework sequence.

Preferably, the CDR sequences of the VH and/or VL according to the above aspects are according to the Kabat definition. Alternatively, the CDRs of the VH and/or VL according to the above aspects are according to the Chothia definition. Further alternatively, the CDRs of the VH and/or VL according to the above aspects are according to the Contact definition. Still further alternatively, the CDRs of the VH and/or VL according to the above aspects are according to the IMGT definition.

In one aspect, the second antigen binding domain comprises a heavy chain variable region (VH_{IL-33}) comprising the amino acid sequence of SEQ ID NO: 12; and a light chain variable region (VL_{IL-33}) comprising the amino acid sequence of SEQ ID NO: 16.

In one aspect, the second antigen binding domain comprises a heavy chain variable region (VH_{IL-33}) comprising the heavy chain CDR sequences of 1E1v8, and a light chain variable region (VL_{IL-33}) comprising the light chain CDR sequences of 1E1v8. In one aspect, the first antigen binding domain comprises a heavy chain variable region (VH_{IL-33}) comprising the VH sequence of 1E1v8, and a light chain variable region (VL_{IL-33}) comprising the VL sequence of 1E1v8.

In one aspect, the bispecific antibody comprises not more than one antigen binding domain that binds to IL-33. Accordingly, in such aspect, the bispecific antibody provides monovalent binding to IL-33.

In one aspect, the second antigen binding domain is an antibody fragment selected from the group of an Fv molecule, a scFv molecule and a Fab molecule. In a particular aspect, the second antigen binding domain is a Fab molecule.

In one aspect, the second antigen binding domain is a conventional Fab molecule. In such aspect, the first antigen binding domain is preferably a crossover Fab molecule as described herein, i.e. a Fab molecule wherein the variable domains VH and VL or the constant domains CH1 and CL of the Fab heavy and light chains are exchanged/replaced by each other.

In a preferred aspect, the first antigen binding domain is a Fab molecule wherein the variable domains VL and VH or the constant domains CL and CH1, particularly the variable domains VL and VH, of the Fab light chain and the Fab heavy chain are replaced by each other (i.e. according to such aspect, the first antigen binding domain is a crossover Fab molecule wherein the variable or constant domains of the Fab light chain and the Fab heavy chain are exchanged), and the second antigen binding domain is a conventional Fab molecule.

In one aspect, the second antigen binding domain comprises a human constant region. In one aspect, the second antigen binding domain is a Fab molecule comprising a human constant region, particularly a human CH1 and/or CL domain. In one aspect, the second antigen binding domain comprises a light chain constant region comprising an amino acid sequence that is at least 95%, at least 96%, at least 97%, at least 98%, at least 99% or 100% identical to the amino acid sequence of SEQ ID NO: 40. Particularly, in such aspect, the light chain constant region may comprise amino acid mutations as described herein under “charge modifications”. In some aspects, the second antigen binding domain comprises a heavy chain constant region comprising an amino acid sequence that is at least 95%, at least 96%, at least 97%, at least 98%, at least 99% or 100% identical to the CH1 domain sequence comprised in the amino acid sequence of SEQ ID NO: 42. In particular aspects, the heavy chain constant region (specifically CH1 domain) may comprise amino acid mutations as described herein under “charge modifications”.

### 3. Charge Modifications

The bispecific antibody of the invention may comprise amino acid substitutions in Fab molecules comprised therein which are particularly efficient in reducing mispairing of light chains with non-matching heavy chains (Bence-Jones-type side products), which can occur in the production of Fab-based bispecific antibodies with a VH/VL exchange in one of their binding arms (see also PCT publication no. WO 2015/150447, particularly the examples therein, incorporated herein by reference in its entirety). The ratio of a desired bispecific antibody compared to undesired side products, in particular Bence Jones-type side products occurring in bispecific antibodies with a VH/VL domain exchange in one of their binding arms, can be improved by the introduction of charged amino acids with opposite charges at specific amino acid positions in the CH1 and CL domains (sometimes referred to herein as “charge modifications”).

Accordingly, in some aspects wherein the first and the second antigen binding domain of the bispecific antibody are both Fab molecules, and in one of the antigen binding domains (particularly the first antigen binding domain) the variable domains VL and VH of the Fab light chain and the Fab heavy chain are replaced by each other,
i) in the constant domain CL of the second antigen binding domain the amino acid at position 124 is substituted by a positively charged amino acid (numbering according to Kabat), and wherein in the constant domain CH1 of the second antigen binding domain the amino acid at position 147 or the amino acid at position 213 is substituted by a negatively charged amino acid (numbering according to Kabat EU index); or
ii) in the constant domain CL of the first antigen binding domain the amino acid at position 124 is substituted by a positively charged amino acid (numbering according to Kabat), and wherein in the constant domain CH1 of the first antigen binding domain the amino acid at position 147 or the amino acid at position 213 is substituted by a negatively charged amino acid (numbering according to Kabat EU index).

The bispecific antibody does not comprise both modifications mentioned under i) and ii). The constant domains CL and CH1 of the antigen binding domain having the VH/VL exchange are not replaced by each other (i.e. remain unexchanged).

In a more specific aspect,
i) in the constant domain CL of the second antigen binding domain the amino acid at position 124 is substituted independently by lysine (K), arginine (R) or histidine (H) (numbering according to Kabat), and in the constant domain CH1 of the second antigen binding domain the amino acid at position 147 or the amino acid at position 213 is substituted independently by glutamic acid (E), or aspartic acid (D) (numbering according to Kabat EU index); or
ii) in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 or the amino acid at position 213 is substituted independently by glutamic acid (E), or aspartic acid (D) (numbering according to Kabat EU index).

In one such aspect, in the constant domain CL of the second antigen binding domain the amino acid at position 124 is substituted independently by lysine (K), arginine (R) or histidine (H) (numbering according to Kabat), and in the constant domain CH1 of the second antigen binding domain the amino acid at position 147 or the amino acid at position 213 is substituted independently by glutamic acid (E), or aspartic acid (D) (numbering according to Kabat EU index).

In a further aspect, in the constant domain CL of the second antigen binding domain the amino acid at position 124 is substituted independently by lysine (K), arginine (R) or histidine (H) (numbering according to Kabat), and in the constant domain CH1 of the second antigen binding domain the amino acid at position 147 is substituted independently by glutamic acid (E), or aspartic acid (D) (numbering according to Kabat EU index).

In a preferred aspect, in the constant domain CL of the second antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more preferred aspect, in the constant domain CL of the second antigen binding domain the amino acid at position 124 is substituted by lysine (K) (numbering according to Kabat) and the amino acid at position 123 is substituted by lysine (K) (numbering according to Kabat), and in the constant domain CH1 of the second antigen binding domain the amino acid at position 147 is substituted by glutamic acid (E) (numbering according to Kabat EU index) and the amino acid at position 213 is substituted by glutamic acid (E) (numbering according to Kabat EU index).

In an even more preferred aspect, in the constant domain CL of the second antigen binding domain the amino acid at position 124 is substituted by lysine (K) (numbering according to Kabat) and the amino acid at position 123 is substituted by arginine (R) (numbering according to Kabat), and in the constant domain CH1 of the second antigen binding domain the amino acid at position 147 is substituted by glutamic acid (E) (numbering according to Kabat EU index) and the amino acid at position 213 is substituted by glutamic acid (E) (numbering according to Kabat EU index).

In preferred aspects, if amino acid substitutions according to the above aspects are made in the constant domain CL and the constant domain CH1 of the second antigen binding domain, the constant domain CL of the second antigen binding domain is of kappa isotype.

Alternatively, the amino acid substitutions according to the above aspects may be made in the constant domain CL and the constant domain CH1 of the first antigen binding domain instead of in the constant domain CL and the constant domain CH1 of the second antigen binding domain. In preferred such aspects, the constant domain CL of the first antigen binding domain is of kappa isotype.

Accordingly, in one aspect, in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 or the amino acid at position 213 is substituted independently by glutamic acid (E), or aspartic acid (D) (numbering according to Kabat EU index).

In a further aspect, in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 is substituted independently by glutamic acid (E), or aspartic acid (D) (numbering according to Kabat EU index).

In still another aspect, in the constant domain CL of the first antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the first antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the constant domain CL of the first antigen binding domain the amino acid at position 124 is substituted by lysine (K) (numbering according to Kabat) and the amino acid at position 123 is substituted by lysine (K) (numbering according to Kabat), and in the constant domain CH1 of the first antigen binding domain the amino acid at position 147 is substituted by glutamic acid (E) (numbering according to Kabat EU index) and the amino acid at position 213 is substituted by glutamic acid (E) (numbering according to Kabat EU index).

In another aspect, in the constant domain CL of the first antigen binding domain the amino acid at position 124 is substituted by lysine (K) (numbering according to Kabat) and the amino acid at position 123 is substituted by arginine (R) (numbering according to Kabat), and in the constant domain CH1 of the first antigen binding domain the amino acid at position 147 is substituted by glutamic acid (E) (numbering according to Kabat EU index) and the amino acid at position 213 is substituted by glutamic acid (E) (numbering according to Kabat EU index).

In a preferred aspect, the first antigen binding domain of the bispecific antibody of the invention is a crossover Fab molecule wherein the variable domains VH and VL are exchanged/replaced by each other, and the second antigen binding domain of the bispecific antibody of the invention is a conventional Fab molecule wherein in the constant domain CL the amino acid at position 124 is substituted by lysine (K) (numbering according to Kabat) and the amino acid at position 123 is substituted by arginine (R) (numbering according to Kabat), and in the constant domain CH1 the amino acid at position 147 is substituted by glutamic acid (E) (numbering according to Kabat EU index) and the amino acid at position 213 is substituted by glutamic acid (E) (numbering according to Kabat EU index).

### 4. Fc Domain

In preferred aspects, the bispecific antibody of the invention comprises an Fc domain composed of a first and a second subunit. The first and the second subunit of the Fc domain are capable of stable association.

The Fc domain of the bispecific antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In one aspect, the Fc domain of the bispecific antibody is an IgG Fc domain. In a preferred aspect, the Fc domain is an IgG₁ Fc domain. In another aspect, the Fc domain is an IgG₄ Fc domain. In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further preferred aspect, the Fc domain is a human Fc domain. In an even more preferred aspect, the Fc domain is a human IgG₁ Fc domain. An exemplary sequence of a human IgG₁ Fc domain is given in SEQ ID NO: 39. In one aspect, additionally the C-terminal lysine (Lys447) is present. In another aspect, the C-terminal glycine (Gly446) is absent. In such aspect, the C-terminal amino acid residue may be proline (Pro445) or proline amide (Pro445-NH₂).

The C-terminus of the Fc domain of an antibody provided herein may be a complete C-terminus ending with the amino acid residues PGK. The C-terminus of the Fc domain may also be a shortened C-terminus in which one or two of the C terminal amino acid residues have been removed. In one aspect, the C-terminus of the Fc domain is a shortened C-terminus ending with the amino acid residue P. In one aspect, the C-terminus of the Fc domain is a shortened C-terminus ending with the amino acid residues PG. In one aspect, an antibody comprising an Fc domain as specified herein, comprises the C-terminal glycine-lysine dipeptide (G446 and K447, Kabat EU numbering of amino acid positions). In one aspect, an antibody comprising an Fc domain as specified herein, comprises a C-terminal glycine residue (G446, Kabat EU numbering of amino acid positions).

The Fc domain may incorporate any of the modifications, singly or in combination, described hereinbelow in relation to Fc domain variants.

### Fc Domain Variants

### Fc Domain Modifications Promoting Heterodimerization

Bispecific antibodies according to the invention comprise different antigen binding domains, which may be fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of bispecific antibodies in recombinant production, it will thus be advantageous to introduce in the Fc domain of the bispecific antibody a modification promoting the association of the desired polypeptides.

Accordingly, in a preferred aspect, the Fc domain of the bispecific antibody according to the invention comprises a modification, particularly an amino acid substitution of combination of amino acid substitutions, promoting the association of the first and the second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between two first or two second CH3 domains are formed). These different approaches for improved heavy chain heterodimerization are contemplated as different alternatives in combination with the heavy-light chain modifications (e.g. VH and VL exchange/replacement and/or introduction of substitutions of charged amino acids with opposite charges in the CH1/CL interface) in the bispecific antibody which reduce heavy/light chain mispairing and Bence Jones-type side products.

In a specific aspect, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a preferred aspect, in the CH3 domain of the first subunit of the Fc domain of the bispecific antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific aspect, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In one aspect, the antigen binding domain that binds to IL-33 is fused to the first subunit of the Fc domain (comprising the “knob” modification). Without wishing to be bound by theory, fusion of the antigen binding domain that binds IL-33 to the knob-containing subunit of the Fc domain will (further) minimize the generation of antibodies comprising two antigen binding domains that bind to IL-33 (steric clash of two knob-containing polypeptides).

Other techniques of CH3-modification for promoting the heterodimerization of Fc domain subunits are also contemplated according to the invention.

For example, in an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such aspect, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In a further aspect, the first CH3 domain further comprises amino acid substitution of K409 or R409 with a negatively charged amino acid (e.g. glutamic acid (E), or aspartic acid (D), particularly K409D or R409D). In a further aspect the first CH3 domain further or alternatively comprises amino acid substitution of K439 and/or K370 with a negatively charged amino acid (e.g. glutamic acid (E), or aspartic acid (D)) (all numberings according to Kabat EU index).

### Fc Domain Modifications Reducing Fcγ Receptor Binding and/or Effector Function

The Fc domain confers to the bispecific antibody favorable pharmacokinetic properties, including a long serum half-life which contributes to good accumulation in the target tissue and a favorable tissue-blood distribution ratio. At the same time it may, however, lead to undesirable targeting of the bispecific antibody to cells expressing Fc receptors. Moreover, the activation of Fc receptor signaling pathways and immune cells may also lead to undesired effects upon systemic administration.

Accordingly, in a preferred aspect, the Fc domain of the bispecific antibody according to the invention exhibits reduced binding affinity to an Fc receptor and/or reduced effector function, as compared to a native IgG₁ Fc domain. In one such aspect, the Fc domain (or the bispecific antibody comprising said Fc domain) exhibits less than 50%, particularly less than 20%, more particularly less than 10% and most particularly less than 5% of the binding affinity to an Fc receptor, as compared to a native IgG₁ Fc domain (or a bispecific antibody comprising a native IgG₁ Fc domain), and/or less than 50%, particularly less than 20%, more particularly less than 10% and most particularly less than 5% of the effector function, as compared to a native IgG₁ Fc domain (or a bispecific antibody comprising a native IgG₁ Fc domain). In one aspect, the Fc domain (or the bispecific antibody comprising said Fc domain) does not substantially bind to an Fc receptor and/or induce effector function. In a preferred aspect, the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the effector function is one or more selected from the group of CDC, ADCC, ADCP, and cytokine secretion. In a preferred aspect, the effector function is ADCC. In one aspect, the Fc domain exhibits substantially similar binding affinity to neonatal Fc receptor (FcRn), as compared to a native IgG₁ Fc domain. Substantially similar binding to FcRn is achieved when the Fc domain (or the bispecific antibody comprising said Fc domain) exhibits greater than about 70%, particularly greater than about 80%, more particularly greater than about 90% of the binding affinity of a native IgG₁ Fc domain (or the bispecific antibody comprising a native IgG₁ Fc domain) to FcRn.

In one aspect, the Fc domain of the bispecific antibody is engineered to have reduced binding affinity to an Fc receptor and/or reduced effector function, as compared to a non-engineered Fc domain. In a preferred aspect, the Fc domain of the bispecific antibody comprises a modification, particularly an amino acid substitution or combination of amino acid substitutions, that reduces the binding affinity to an Fc receptor and/or effector function of the antibody, as compared to a corresponding antibody comprising an Fc domain without such modification. In one such aspect, the Fc receptor is an Fcγ receptor. In one aspect, the Fc receptor is a human Fc receptor. In one aspect, the Fc receptor is an activating Fc receptor. In a specific such aspect, the Fc receptor is an activating human Fcγ receptor, more specifically human FcγRIIIa, FcγRI or FcγRIIa, most specifically human FcγRIIIa. Typically, the same one or more amino acid mutation is present in each of the two subunits of the Fc domain. In one aspect, the amino acid mutation reduces the binding affinity of the Fc domain to an Fc receptor. In one aspect, the amino acid mutation reduces the binding affinity of the Fc domain to an Fc receptor by at least 2-fold, at least 5-fold, or at least 10-fold. In aspects where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one aspect, the (bispecific) antibody comprising an engineered Fc domain exhibits less than 20%, particularly less than 10%, more particularly less than 5% of the binding affinity to an Fc receptor as compared to a (bispecific) antibody comprising a non-engineered Fc domain. In a preferred aspect, the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In one aspect, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the (bispecific) antibody comprising said Fc domain) exhibits greater than about 70% of the binding affinity of a non-engineered form of the Fc domain (or the (bispecific) antibody comprising said non-engineered form of the Fc domain) to FcRn. The Fc domain, or a (bispecific) antibody comprising said Fc domain, may exhibit greater than about 80% and even greater than about 90% of such affinity. In one aspect, the Fc domain of the (bispecific) antibody is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one aspect, the reduced effector function is one or more selected from the group of reduced CDC, reduced ADCC, reduced ADCP, and reduced cytokine secretion. In a preferred aspect, the reduced effector function is reduced ADCC. In one aspect the reduced ADCC is less than 20% of the ADCC induced by a non-engineered Fc domain (or a (bispecific) antibody comprising a non-engineered Fc domain).

In one aspect, the amino acid mutation that reduces the binding affinity of the Fc domain to an Fc receptor and/or effector function is an amino acid substitution. In one aspect, the Fc domain comprises an amino acid substitution at one or more position selected from the group of E233, L234, L235, N297, P331 and P329 (numberings according to Kabat EU index). In a more specific aspect, the Fc domain comprises an amino acid substitution at one or more position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In a preferred aspect, the Fc domain comprises amino acid substitutions at positions P329, L234 and L235 (numberings according to Kabat EU index). In a more preferred aspect, the Fc domain comprises the amino acid mutations L234A, L235A and P329G (“P329G LALA”, “PGLALA” or “LALAPG”). Specifically, in a preferred aspect, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one such aspect, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT 15 publication no. WO 2012/130831 or in Schlothauer et al., Protein Eng Des Sel 29, 457-466 (2016), which are both incorporated herein by reference in its entirety. WO 2012/130831 and Schlothauer et al. also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some aspects, the Fc domain of the bispecific antibody of the invention is an IgG₄ Fc domain, particularly a human IgG₄ Fc domain. In one aspect, the IgG₄ Fc domain comprises an amino acid substitution at position S228, specifically the amino acid substitution S228P (numberings according to Kabat EU index). To further reduce its binding affinity to an Fc receptor and/or its effector function, in one aspect, the IgG₄ Fc domain comprises an amino acid substitution at position L235, specifically the amino acid substitution L235E (numberings according to Kabat EU index). In another aspect, the IgG₄ Fc domain comprises an amino acid substitution at position P329, specifically the amino acid substitution P329G (numberings according to Kabat EU index). In a preferred aspect, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831 or in Schlothauer et al., Protein Eng Des Sel 29, 457-466 (2016), both incorporated herein by reference in its entirety.

In a preferred aspect, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

Other Fc domain modifications for reducing Fc receptor binding and/or effector function are also contemplated according to the invention.

For example, in one aspect, N-glycosylation of the Fc domain has been eliminated. In one such aspect, the Fc domain comprises an amino acid mutation at position N297, particularly an amino acid substitution replacing asparagine by alanine (N297A), aspartic acid (N297D), glutamine (N297Q) or glycine (N297G) (numberings according to Kabat EU index). In one aspect, the Fc domain further comprises an amino acid substitution at position D265A (numbering according to Kabat EU index). In one aspect, the substitutions are D265A and N297A (DANA), or D265A and N297G (DANG). In such aspect, the Fc domain is a human IgG₁ Fc domain.

In another aspect, the Fc domain, particularly a human IgG₁ Fc domain, comprises the amino acid substitutions E233P, L234V, L235A and the amino acid deletion G236del (see e.g. Armour et al., Eur. J. Immunol. 29, 2613-2624 (1999)). In one aspect, the Fc domain further comprises a N297G or S267K substitution (numberings according to Kabat EU index).

In yet another aspect, the Fc domain, particularly a human IgG₁ Fc domain, comprises the amino acid substitutions L234F, L235E and D265A (FEA), or L234F, L235E and P331S (FES) (numberings according to Kabat EU index).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (Cytiva), and Fc receptors such as may be obtained by recombinant expression. Alternatively, binding affinity of Fc domains or antibodies comprising an Fc domain for Fc receptors may be evaluated using cell lines known to express particular Fc receptors, such as human NK cells expressing FcγIIIa receptor.

Effector function of an Fc domain, or a (bispecific) antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays may be employed (see, for example, ACTI™ non-radioactive cytotoxicity assay for flow cytometry (CellTechnology, Inc. Mountain View, CA); and CYTOTOX 96©non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

In some aspects, binding of the Fc domain to a complement component, specifically to C1q, is reduced. Accordingly, in some aspects wherein the Fc domain is engineered to have reduced effector function, said reduced effector function includes reduced CDC. C1q binding assays may be carried out to determine whether the Fc domain, or the (bispecific) antibody comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

### Fc Domain Modifications Increasing FcRn Binding and/or Serum Half-Life

It may be desirable to further prolong serum half-life of the bispecific antibody of the invention, by introducing modifications in the Fc domain which increase binding to FcRn and/or serum half-life of the antibody. The benefits of such prolonged half-life include increased convenience in therapeutic application in that it allows extending the dosing interval (i.e. the time between subsequent administrations) of the antibody.

Accordingly, in one aspect, the Fc domain of the bispecific antibody according to the invention comprises a modification, particularly an amino acid substitution or combination of amino acid substitutions, that increases the binding affinity to an FcRn receptor and/or the serum half-life of the antibody, as compared to a corresponding antibody comprising an Fc domain without such modification. In a particular aspect, the FcRn receptor is a human FcRn receptor. In one aspect, the Fc domain comprises an amino acid substitution at one or more position selected from the group of 252, 254, 256, 428 and 434 (numberings according to Kabat EU index).

In a more specific aspect, the Fc domain comprises an amino acid substitution at one or more position selected from the group of 252, 254 and 256 (numberings according to Kabat EU index).

In a preferred aspect, the Fc domain (in each of its subunits) comprises the amino acid substitutions M252Y, S254T and T256E (“YTE”; numberings according to Kabat EU index). See, e.g., Dall'Acqua et al. J Biol Chem 281, 23514-23524 (2006) or WO 2002/60919 (both incorporated by reference herein in their entirety). In such aspect, the Fc domain is an IgG₁ Fc domain, particularly a human IgG₁ Fc domain.

In another aspect, the Fc domain comprises an amino acid substitution at one or more position selected from the group of 428 and 434 (numberings according to Kabat EU index). In a preferred aspect, the Fc domain (in each of its subunits) comprises the amino acid substitutions M428L and N434S (“LS”; numberings according to Kabat EU index). See e.g. Zalevsky et al. Nat Biotech 28, 157-159 (2010) or WO 2009/086320 (both incorporated by reference herein in their entirety). In one aspect, the Fc domain (in each of its subunits) comprises the amino acid substitutions M428L and N434A (“LA”; numberings according to Kabat EU index). In such aspects, the Fc domain is an IgG₁ Fc domain, particularly a human IgG₁ Fc domain.

Fc domain modifications that increase FcRn binding and/or serum half-life may be combined with modifications that reduce binding to rheumatoid factor. Such modifications include e.g. amino acid substitutions at positions 424, 436, 438 and/or 440 (Kabat EU numbering of residues). Accordingly, in one aspect, the Fc domain of the bispecific antibody according to the invention comprises (i) an amino acid mutation, particularly an amino acid substitution, that increases the binding affinity of the Fc domain to an FcRn receptor and/or the serum half-life of the antibody as described hereinabove, and (ii) an amino acid mutation, particularly an amino acid substitution, that reduces the binding affinity of the Fc domain to rheumatoid factor. In a particular aspect, the rheumatoid factor is human rheumatoid factor.

In one aspect, the Fc domain (in each of its subunits) comprises the amino acid substitutions M428L, N434A and Y436T, or M428L, N434S and Y436T (numberings according to Kabat EU index). In one aspect, the Fc domain (in each of its subunits) comprises the amino acid substitutions N434A, Q438R, S440E, and optionally Y436T or Y436V (numberings according to Kabat EU index). In one aspect, the Fc domain (in each of its subunits) comprises the amino acid substitutions M428L, N434A, Q438R, S440E, and optionally Y436T or Y436V (numberings according to Kabat EU index). See Maeda et al. MABS 9, 844-853 (2017) (incorporated herein by reference in its entirety). In such aspects, the Fc domain is an IgG₁ Fc domain, particularly a human IgG₁ Fc domain.

FcRn binding and in vivo clearance/half-life determinations can also be performed using methods known in the art (see e.g., Petkova, S. B. et al., Int'l. Immunol. 18(12):1759-1769 (2006); WO 2013/120929).

In a preferred aspect, the Fc domain of the bispecific antibody of the invention is a human IgG₁ Fc domain comprising
(i) in the first subunit the amino acid substitutions S354C and T366W, and in the second subunit the amino acid substitutions Y349C, T366S, L368A and Y407V;
(ii) in each of its subunits the amino acid substitutions L234A, L235A and P329G; and optionally
(iii) in each of its subunits the amino acid substitutions M252Y, S254T and T256E, or the amino acid substitutions M428L and N434S (numberings according to Kabat EU index).

### 5. Bispecific Antibody Formats

The bispecific antibody according to the invention can have different configurations, i.e. the first and second antigen binding domain may be fused to each other and/or to the Fc domain in different ways. The components may be fused to each other directly or, preferably, via one or more suitable peptide linkers. Where fusion of a Fab molecule is to the N-terminus of a subunit of the Fc domain, it is typically via an immunoglobulin hinge region.

In a preferred aspect, the anti-IL-33/anti-IL-6 bispecific antibody of the invention comprises
(a) a first antigen binding domain that binds to IL-6,
(b) a second antigen binding domain that binds to IL-33, and
(c) an Fc domain composed of a first and a second subunit.

In one aspect, the first and the second antigen binding domain are each a Fab molecule and are each fused at the C-terminus of the Fab heavy chain to the N-terminus of one of the subunits of the Fc domain. In a particular such aspect, the first antigen binding domain is fused at the C-terminus of the Fab heavy chain to the N-terminus of the second subunit of the Fc domain, and the second antigen binding domain is fused at the C-terminus of the Fab heavy chain to the N-terminus of the first subunit of the Fc domain. In another such aspect, the first antigen binding domain is fused at the C-terminus of the Fab heavy chain to the N-terminus of the first subunit of the Fc domain, and the second antigen binding domain is fused at the C-terminus of the Fab heavy chain to the N-terminus of the second subunit of the Fc domain.

In a preferred aspect, the first antigen binding domain is a crossover Fab molecule as described herein, i.e. a Fab molecule wherein the variable domains VH and VL or the constant domains CL and CH1 of the Fab heavy and light chains are exchanged/replaced by each other, and the second antigen binding domain is a conventional Fab molecule. In a specific such aspect, the first antigen binding domain is a crossover Fab molecule wherein the variable domains VH and VL are exchanged/replaced by each other, and the second antigen binding domain is a conventional Fab molecule optionally comprising charge modifications as described herein.

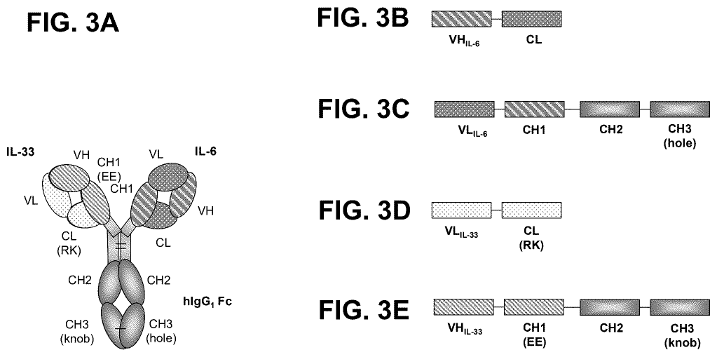

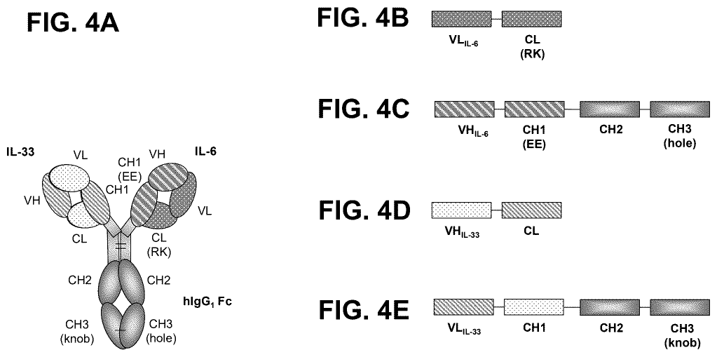

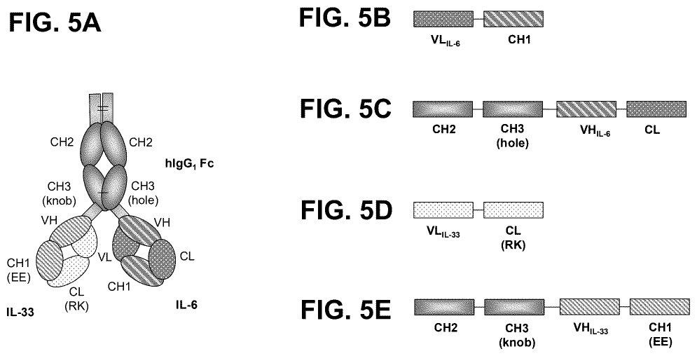

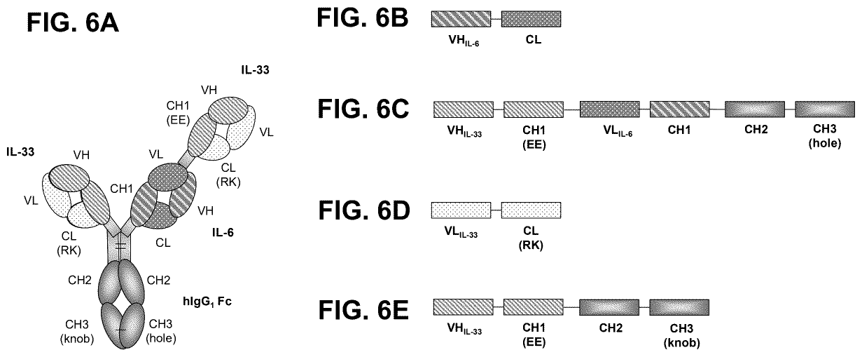

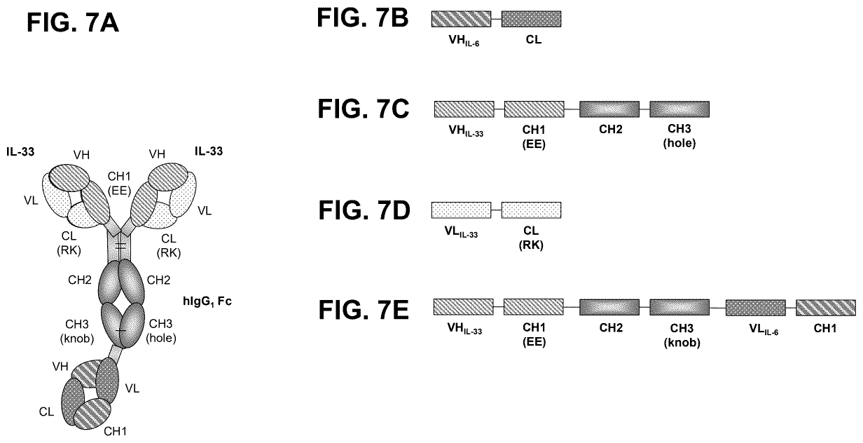

Such a configuration is schematically depicted in FIGS. 3A-3E.

In one aspect, the bispecific antibody essentially consists of the first and the second antigen binding domain, the Fc domain composed of a first and a second subunit, and optionally one or more peptide linkers.

The first and the second antigen binding domain may be fused to the Fc domain directly or through a peptide linker. Various linkers may be used, particularly peptide linkers comprising one or more amino acids, typically about 2-20 amino acids, that are described herein or are known in the art.

Suitable, non-immunogenic peptide linkers include, for example, (G₄S)ₙ peptide linkers, wherein n is generally an integer from 1 to 10, typically from 1 to 4. Additionally or alternatively, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a preferred aspect the first and the second antigen binding domain (particularly wherein the first and the second antigen binding domain are Fab molecules) are each fused to the Fc domain through an immunoglobulin hinge region. In a specific aspect, the immunoglobulin hinge region is a human IgG₁ hinge region, particularly where the Fc domain is an IgG₁ Fc domain.

In one aspect, the first and the second antigen binding domain are each a Fab molecule and together with the Fc domain form part of an immunoglobulin molecule. For clarity, if one of the Fab molecules is a crossover Fab molecule as described herein, the immunoglobulin molecule is a crossover immunoglobulin molecule wherein in one of its Fab molecules the variable domains VH and VL or the constant domains CL and CH1 are exchanged/replaced by each other.

In a preferred aspect, the immunoglobulin molecule is an IgG class immunoglobulin. In an even more preferred aspect, the immunoglobulin is an IgG₁ subclass immunoglobulin. In another aspect the immunoglobulin is an IgG₄ subclass immunoglobulin. In a further preferred aspect, the immunoglobulin is a humanized immunoglobulin. In another aspect, the immunoglobulin is a human immunoglobulin. In one aspect, the immunoglobulin comprises a human constant region, particularly a human Fc domain. In one aspect, the immunoglobulin molecule is an IgG class, particularly an IgG₁ subclass, immunoglobulin molecule comprising a human CH1, CH2, CH3 and/or CL domain. Exemplary sequences of human constant domains are given in SEQ ID NOs 40 and 41 (human kappa and lambda CL domains, respectively) and SEQ ID NO: 42 (human IgG₁ heavy chain constant domains CH1-CH2-CH3). In one aspect, the immunoglobulin molecule comprises a light chain constant region comprising an amino acid sequence that is at least about 95%, 96%, 97%, 98%, 99% or 100% identical to the amino acid sequence of SEQ ID NO: 40 or SEQ ID NO: 41. In one aspect, the immunoglobulin molecule comprises a heavy chain constant region comprising an amino acid sequence that is at least about 95%, 96%, 97%, 98%, 99% or 100% identical to the amino acid sequence of SEQ ID NO: 42. Particularly, the heavy chain constant region may comprise amino acid mutations in the Fc domain as described herein.

In one aspect, the bispecific antibody is a full-length antibody, particularly a full-length IgG antibody. For clarity, if one of the antigen binding domains of the bispecific antibody is a crossover Fab molecule as described herein, the full-length antibody is a crossover antibody wherein in one of its binding arms the variable domains VH and VL or the constant domains CL and CH1 are exchanged/replaced by each other.

In a preferred aspect, the full-length antibody is an IgG antibody. In an even more preferred aspect, the full-length antibody is an IgG₁ antibody. In another aspect, the full-length antibody is an IgG₄ antibody. In a further preferred aspect, the full-length antibody is a humanized antibody. In another aspect, the full-length antibody is a human antibody. In one aspect, the full-length antibody comprises a human constant region, particularly a human Fc domain. In one aspect, the full-length antibody comprises a human constant region, particularly a human Fc domain. In one aspect, the full-length antibody molecule is an IgG, particularly an IgG₁, antibody comprising a human CH1, CH2, CH3 and/or CL domain. Exemplary sequences of human constant domains are given in SEQ ID NOs 40 and 41 (human kappa and lambda CL domains, respectively) and SEQ ID NO: 42 (human IgG₁ heavy chain constant domains CH1-CH2-CH3). In one aspect, the full-length antibody comprises a light chain constant region comprising an amino acid sequence that is at least about 95%, 96%, 97%, 98%, 99% or 100% identical to the amino acid sequence of SEQ ID NO: 40 or SEQ ID NO: 41. In one aspect, the full-length antibody comprises a heavy chain constant region comprising an amino acid sequence that is at least about 95%, 96%, 97%, 98%, 99% or 100% identical to the amino acid sequence of SEQ ID NO: 42. Particularly, the heavy chain constant region may comprise amino acid mutations in the Fc domain as described herein.

In some embodiments, the anti-IL-33/anti-IL-6 bispecific antibody comprises (e.g., essentially consists of) (i) a first polypeptide comprising from the N-terminus to the C-terminus: a VH domain of a first antigen binding domain that binds to IL-6, and a CL domain; (ii) a second polypeptide comprising from the N-terminus to the C-terminus: a VL domain of the first antigen binding domain that binds to IL-6, a CH1 domain, a CH2 domain and a CH3 domain; (iii) a third polypeptide comprising from the N-terminus to the C-terminus: a VL domain of a second antigen binding domain that binds to IL-33, and a CL domain; and (iv) a fourth polypeptide comprising from the N-terminus to the C-terminus: a VH domain of the second antigen binding domain that binds to IL-33, a CH1 domain, a CH2 domain and a CH3 domain.

In a specific aspect, the anti-IL-33/anti-IL-6 bispecific antibody of the invention comprises (a) a first antigen binding domain that binds to IL-6, comprising (i) a heavy chain variable region (VH_{IL-6}) comprising the HCDR 1 of SEQ ID NO: 1, the HCDR 2 of SEQ ID NO: 2, and the HCDR 3 of SEQ ID NO: 3, and a light chain variable region (VL_{IL-6}) comprising the LCDR 1 of SEQ ID NO: 5, the LCDR 2 of SEQ ID NO: 6 and the LCDR 3 of SEQ ID NO: 7, and/or (ii) a heavy chain variable region (VH_{IL-6}) comprising the amino acid sequence of SEQ ID NO: 4; and a light chain variable region (VL_{IL-6}) comprising the amino acid sequence of SEQ ID NO: 8;
(b) a second antigen binding domain that binds to IL-33, comprising a heavy chain variable region (VH_{IL-33}) comprising the HCDR 1 of SEQ ID NO: 9, the HCDR 2 of SEQ ID NO: 10, and the HCDR 3 of SEQ ID NO: 11, and a light chain variable region (VL_{IL-33}) comprising the LCDR 1 of SEQ ID NO: 13, the LCDR 2 of SEQ ID NO: 14 and the LCDR 3 of SEQ ID NO: 15, and/or (ii) a heavy chain variable region (VH_{IL-33}) comprising the amino acid sequence of SEQ ID NO: 12; and a light chain variable region (VL_{IL-33}) comprising the amino acid sequence of SEQ ID NO: 16; and
(c) an Fc domain composed of a first and a second subunit;
wherein the first and the second antigen binding domain are each a Fab molecule and are each fused at the C-terminus of the Fab heavy chain to the N-terminus of one of the subunits of the Fc domain. In a particular such aspect, the first antigen binding domain is fused at the C-terminus of the Fab heavy chain to the N-terminus of the second subunit of the Fc domain, and the second antigen binding domain is fused at the C-terminus of the Fab heavy chain to the N-terminus of the first subunit of the Fc domain. In another such aspect, the first antigen binding domain is fused at the C-terminus of the Fab heavy chain to the N-terminus of the first subunit of the Fc domain, and the second antigen binding domain is fused at the C-terminus of the Fab heavy chain to the N-terminus of the second subunit of the Fc domain.

In a more specific aspect, the first antigen binding domain is a Fab molecule wherein the variable domains VL and VH or the constant domains CL and CH1, particularly the variable domains VL and VH, of the Fab light chain and the Fab heavy chain are replaced by each other, and the second antigen binding domain is a conventional Fab molecule. In an even more specific aspect, the first antigen binding domain is a crossover Fab molecule wherein the variable domains VH and VL are replaced by each other, and the second antigen binding domain is a conventional Fab molecule wherein in the constant domain CL the amino acid at position 124 is substituted by lysine (K) (numbering according to Kabat) and the amino acid at position 123 is substituted by arginine (R) (numbering according to Kabat), and in the constant domain CH1 the amino acid at position 147 is substituted by glutamic acid (E) (numbering according to Kabat EU index) and the amino acid at position 213 is substituted by glutamic acid (E) (numbering according to Kabat EU index).

In a further specific aspect, the Fc domain is a human IgG₁ Fc domain. In one aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index). In a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). In still a further aspect, in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In still a further aspect, in each of the first and the second subunit of the Fc domain (i) the methionine residue at position 252 is replaced by a tyrosine residue (M252Y), the serine residue at position 254 is replaced by a threonine residue (S254T) and the threonine residue at position 256 is replaced by a glutamic acid residue (T256E), or (ii) the methionine residue at position 428 is replaced by a leucine residue (M428L) and the asparagine residue at position 434 is replaced by a serine residue (N434S) (numbering according to Kabat EU index).

In a more specific aspect, the Fc domain of the bispecific antibody of the invention is a human IgG1 Fc domain comprising
(i) in the first subunit the amino acid substitutions S354C and T366W, and in the second subunit the amino acid substitutions Y349C, T366S, L368A and Y407V;
(ii) in each of its subunits the amino acid substitutions L234A, L235A and P329G; and optionally
(iii) in each of its subunits the amino acid substitutions M252Y, S254T and T256E, or the amino acid substitutions M428L and N434S (numberings according to Kabat EU index).

In one aspect, the anti-IL-33/anti-IL-6 bispecific antibody of the invention comprises a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 24 or SEQ ID NO: 26. In one aspect, the bispecific antibody comprises a HC1 comprising an amino acid sequence having at least 95% sequence identity to the amino acid sequence of SEQ ID NO: 24 or SEQ ID NO: 26. In one aspect, the bispecific antibody comprises a HC1 comprising an amino acid sequence having at least 98% sequence identity to the amino acid sequence of SEQ ID NO: 24 or SEQ ID NO: 26. In one aspect, the bispecific antibody comprises a HC1 comprising the amino acid sequence of SEQ ID NO: 24 or SEQ ID NO: 26 comprising up to two (i.e. 0, 1 or 2) amino acid substitutions within said amino acid sequence. In one aspect, the bispecific antibody comprises a HC1 comprising the amino acid sequence of SEQ ID NO: 24 or SEQ ID NO: 26 comprising one amino acid substitution within said amino acid sequence. In one aspect, the bispecific antibody comprises a HC1 comprising the amino acid sequence of SEQ ID NO: 24 or SEQ ID NO: 26 comprising two amino acid substitutions within said amino acid sequence. In one aspect, the bispecific antibody comprises a HC1 comprising the amino acid sequence of SEQ ID NO: 24 or SEQ ID NO: 26.

In one aspect, the anti-IL-33/anti-IL-6 bispecific antibody of the invention comprises a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 20. In one aspect, the bispecific antibody comprises a LC1 comprising an amino acid sequence having at least 95% sequence identity to the amino acid sequence of SEQ ID NO: 20. In one aspect, the bispecific antibody comprises a LC1 comprising an amino acid sequence having at least 98% sequence identity to the amino acid sequence of SEQ ID NO: 20. In one aspect, the bispecific antibody comprises a LC1 comprising the amino acid sequence of SEQ ID NO: 20 comprising up to two (i.e. 0, 1 or 2) amino acid substitutions within said amino acid sequence. In one aspect, the bispecific antibody comprises a LC1 comprising the amino acid sequence of SEQ ID NO: 20 comprising one amino acid substitution within said amino acid sequence. In one aspect, the bispecific antibody comprises a LC1 comprising the amino acid sequence of SEQ ID NO: 20 comprising two amino acid substitutions within said amino acid sequence. In one aspect, the bispecific antibody comprises a LC1 comprising the amino acid sequence of SEQ ID NO: 20.

In particular aspects, the anti-IL-33/anti-IL-6 bispecific antibody of the invention comprises a HC1 as in any of the aspects provided above and a LC1 as in any of the aspects provided above.

In one aspect, the anti-IL-33/anti-IL-6 bispecific antibody of the invention comprises a HC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 24 or SEQ ID NO: 26, and a LC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 20. In one aspect, the bispecific antibody comprises a HC1 comprising an amino acid sequence having at least 95% sequence identity to the amino acid sequence of SEQ ID NO: 24 or SEQ ID NO: 26, and a LC1 comprising an amino acid sequence having at least 95% sequence identity to the amino acid sequence of SEQ ID NO: 20. In one aspect, the bispecific antibody comprises a HC1 comprising an amino acid sequence having at least 98% sequence identity to the amino acid sequence of SEQ ID NO: 24 or SEQ ID NO: 26, and a LC1 comprising an amino acid sequence having at least 98% sequence identity to the amino acid sequence of SEQ ID NO: 20. In one aspect, the bispecific antibody comprises a HC1 comprising the amino acid sequence of SEQ ID NO: 24 or SEQ ID NO: 26 comprising up to two (i.e. 0, 1 or 2) amino acid substitutions within said amino acid sequence, and a LC1 comprising the amino acid sequence of SEQ ID NO: 20 comprising up to two (i.e. 0, 1 or 2) amino acid substitutions within said amino acid sequence. In one aspect, the bispecific antibody comprises a HC1 comprising the amino acid sequence of SEQ ID NO: 24 or SEQ ID NO: 26 comprising one amino acid substitution within said amino acid sequence, and a LC1 comprising the amino acid sequence of SEQ ID NO: 20 comprising one amino acid substitution within said amino acid sequence. In one aspect, the bispecific antibody comprises a HC1 comprising the amino acid sequence of SEQ ID NO: 24 or SEQ ID NO: 26 comprising two amino acid substitutions within said amino acid sequence, and a LC1 comprising the amino acid sequence of SEQ ID NO: 20 comprising two amino acid substitutions within said amino acid sequence. In one aspect, the bispecific antibody comprises a HC1 comprising the amino acid sequence of SEQ ID NO: 24 or SEQ ID NO: 26, and a LC1 comprising the amino acid sequence of SEQ ID NO: 20.

In one aspect, the anti-IL-33/anti-IL-6 bispecific antibody of the invention comprises a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23 or SEQ ID NO: 25. In one aspect, the bispecific antibody comprises a HC2 comprising an amino acid sequence having at least 95% sequence identity to the amino acid sequence of SEQ ID NO: 23 or SEQ ID NO: 25. In one aspect, the bispecific antibody comprises a HC2 comprising an amino acid sequence having at least 98% sequence identity to the amino acid sequence of SEQ ID NO: 23 or SEQ ID NO: 25. In one aspect, the bispecific antibody comprises a HC2 comprising the amino acid sequence of SEQ ID NO: 23 or SEQ ID NO: 25 comprising up to two (i.e. 0, 1 or 2) amino acid substitutions within said amino acid sequence. In one aspect, the bispecific antibody comprises a HC2 comprising the amino acid sequence of SEQ ID NO: 23 or SEQ ID NO: 25 comprising one amino acid substitution within said amino acid sequence. In one aspect, the bispecific antibody comprises a HC2 comprising the amino acid sequence of SEQ ID NO: 23 or SEQ ID NO: 25 comprising two amino acid substitutions within said amino acid sequence. In one aspect, the bispecific antibody comprises a HC2 comprising the amino acid sequence of SEQ ID NO: 23 or SEQ ID NO: 25.

In one aspect, the anti-IL-33/anti-IL-6 bispecific antibody of the invention comprises a second light chain (LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 17. In one aspect, the bispecific antibody comprises a LC2 comprising an amino acid sequence having at least 95% sequence identity to the amino acid sequence of SEQ ID NO: 17. In one aspect, the bispecific antibody comprises a LC2 comprising an amino acid sequence having at least 98% sequence identity to the amino acid sequence of SEQ ID NO: 17. In one aspect, the bispecific antibody comprises a LC2 comprising the amino acid sequence of SEQ ID NO: 17 comprising up to two (i.e. 0, 1 or 2) amino acid substitutions within said amino acid sequence. In one aspect, the bispecific antibody comprises a LC2 comprising the amino acid sequence of SEQ ID NO: 17 comprising one amino acid substitution within said amino acid sequence. In one aspect, the bispecific antibody comprises a LC2 comprising the amino acid sequence of SEQ ID NO: 17 comprising two amino acid substitutions within said amino acid sequence. In one aspect, the bispecific antibody comprises a LC2 comprising the amino acid sequence of SEQ ID NO: 17.

In particular aspects, the anti-IL-33/anti-IL-6 bispecific antibody of the invention comprises a HC2 as in any of the aspects provided above and a LC2 as in any of the aspects provided above.

In one aspect, the anti-IL-33/anti-IL-6 bispecific antibody of the invention comprises a HC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23 or SEQ ID NO: 25, and a LC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 17. In one aspect, the bispecific antibody comprises a HC2 comprising an amino acid sequence having at least 95% sequence identity to the amino acid sequence of SEQ ID NO: 23 or SEQ ID NO: 25, and a LC2 comprising an amino acid sequence having at least 95% sequence identity to the amino acid sequence of SEQ ID NO: 17. In one aspect, the bispecific antibody comprises a HC2 comprising an amino acid sequence having at least 98% sequence identity to the amino acid sequence of SEQ ID NO: 23 or SEQ ID NO: 25, and a LC2 comprising an amino acid sequence having at least 98% sequence identity to the amino acid sequence of SEQ ID NO: 17. In one aspect, the bispecific antibody comprises a HC2 comprising the amino acid sequence of SEQ ID NO: 23 or SEQ ID NO: 25 comprising up to two (i.e. 0, 1 or 2) amino acid substitutions within said amino acid sequence, and a LC2 comprising the amino acid sequence of SEQ ID NO: 17 comprising up to two (i.e. 0, 1 or 2) amino acid substitutions within said amino acid sequence. In one aspect, the bispecific antibody comprises a HC2 comprising the amino acid sequence of SEQ ID NO: 23 or SEQ ID NO: 25 comprising one amino acid substitution within said amino acid sequence, and a LC2 comprising the amino acid sequence of SEQ ID NO: 17 comprising one amino acid substitution within said amino acid sequence. In one aspect, the bispecific antibody comprises a HC2 comprising the amino acid sequence of SEQ ID NO: 23 or SEQ ID NO: 25 comprising two amino acid substitutions within said amino acid sequence, and a LC2 comprising the amino acid sequence of SEQ ID NO: 17 comprising two amino acid substitutions within said amino acid sequence. In one aspect, the bispecific antibody comprises a HC2 comprising the amino acid sequence of SEQ ID NO: 23 or SEQ ID NO: 25, and a LC2 comprising the amino acid sequence of SEQ ID NO: 17.

In particular aspects, the anti-IL-33/anti-IL-6 bispecific antibody of the invention comprises a HC1 as in any of the aspects provided above, a LC1 as in any of the aspects provided above, a HC2 as in any of the aspects provided above, and a LC2 as in any of the aspects provided above.

In one aspect, the anti-IL-33/anti-IL-6 bispecific antibody of the invention comprises a HC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 24, a LC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 20, a HC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO:23, and a LC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 17. In one aspect, the bispecific antibody comprises a HC1 comprising the amino acid sequence of SEQ ID NO: 24 comprising up to two (i.e. 0, 1 or 2) amino acid substitutions within said amino acid sequence, a LC1 comprising the amino acid sequence of SEQ ID NO: 20 comprising up to two (i.e. 0, 1 or 2) amino acid substitutions within said amino acid sequence, a HC2 comprising the amino acid sequence of SEQ ID NO: 23 comprising up to two (i.e. 0, 1 or 2) amino acid substitutions within said amino acid sequence, and a LC2 comprising the amino acid sequence of SEQ ID NO: 17 comprising up to two (i.e. 0, 1 or 2) amino acid substitutions within said amino acid sequence. In one aspect, the bispecific antibody comprises a HC1 comprising the amino acid sequence of SEQ ID NO: 24, a LC1 comprising the amino acid sequence of SEQ ID NO: 20, a HC2 comprising the amino acid sequence of SEQ ID NO: 23, and a LC2 comprising the amino acid sequence of SEQ ID NO: 17.

In one aspect, the anti-IL-33/anti-IL-6 bispecific antibody of the invention comprises a HC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26, a LC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 20, a HC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO:25, and a LC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 17. In one aspect, the bispecific antibody comprises a HC1 comprising the amino acid sequence of SEQ ID NO: 26 comprising up to two (i.e. 0, 1 or 2) amino acid substitutions within said amino acid sequence, a LC1 comprising the amino acid sequence of SEQ ID NO: 20 comprising up to two (i.e. 0, 1 or 2) amino acid substitutions within said amino acid sequence, a HC2 comprising the amino acid sequence of SEQ ID NO: 25 comprising up to two (i.e. 0, 1 or 2) amino acid substitutions within said amino acid sequence, and a LC2 comprising the amino acid sequence of SEQ ID NO: 17 comprising up to two (i.e. 0, 1 or 2) amino acid substitutions within said amino acid sequence. In one aspect, the bispecific antibody comprises a HC1 comprising the amino acid sequence of SEQ ID NO: 26, a LC1 comprising the amino acid sequence of SEQ ID NO: 20, a HC2 comprising the amino acid sequence of SEQ ID NO: 25, and a LC2 comprising the amino acid sequence of SEQ ID NO: 17.

In one aspect, the anti-IL-33/anti-IL-6 bispecific antibody of the invention comprises a HC1 comprising the amino acid sequence of SEQ ID NO: 24 or SEQ ID NO: 26 and additionally a C-terminal lysine residue (Lys447; Kabat EU numbering). In another aspect, the bispecific antibody comprises a HC1 comprising the amino acid sequence of SEQ ID NO: 24 or SEQ ID NO: 26 without the C-terminal glycine residue (Gly446; Kabat EU numbering). In such aspect, the C-terminal amino acid residue may be proline (Pro445; Kabat EU numbering) or proline amide (Pro445-NH₂; Kabat EU numbering).

In one aspect, the anti-IL-33/anti-IL-6 bispecific antibody of the invention comprises a HC2 comprising the amino acid sequence of SEQ ID NO: 23 or SEQ ID NO: 25 and additionally a C-terminal lysine residue (Lys447; Kabat EU numbering). In another aspect, the bispecific antibody comprises a HC2 comprising the amino acid sequence of SEQ ID NO: 23 or SEQ ID NO: 25 without the C-terminal glycine residue (Gly446; Kabat EU numbering). In such aspect, the C-terminal amino acid residue may be proline (Pro445; Kabat EU numbering) or proline amide (Pro445-NH₂; Kabat EU numbering).

For clarity, SEQ ID NO: 24 and SEQ ID NO: 26 are referred to as “heavy chain” despite comprising a VL region of a crossover Fab molecule, as defined herein. Conversely, SEQ ID NO: 20 is referred to as “light chain” despite comprising a VH region of a crossover Fab molecule, as defined herein.

In one aspect, the invention provides a bispecific antibody that binds IL-33 and IL-6, comprising a first polypeptide comprising the amino acid sequence of SEQ ID NO: 17, a second polypeptide comprising the amino acid sequence of SEQ ID NO: 20, a third polypeptide comprising the amino acid sequence of SEQ ID NO: 23, and a fourth polypeptide comprising the amino acid sequence of SEQ ID NO: 24.

### Polynucleotides

The invention further provides an isolated polynucleotide encoding an anti-IL-33/anti-IL-6 bispecific antibody of the invention. Said isolated polynucleotide may be a single polynucleotide or a plurality of polynucleotides.

The polynucleotides encoding a bispecific antibody of the invention may be expressed as a single polynucleotide that encodes the entire antibody or as multiple (e.g., two or more) polynucleotides that are co-expressed. Polypeptides encoded by polynucleotides that are co-expressed may associate through, e.g., disulfide bonds or other means to form a functional antibody. For example, the light chain portion of an antibody may be encoded by a separate polynucleotide from the portion of the antibody comprising the heavy chain of the antibody. When co-expressed, the heavy chain polypeptides will associate with the light chain polypeptides to form the antibody. In another example, the portion of the antibody comprising one of the two Fc domain subunits and optionally (part of) one or more Fab molecules could be encoded by a separate polynucleotide from the portion of the antibody comprising the other of the two Fc domain subunits and optionally (part of) a Fab molecule. When co-expressed, the Fc domain subunits will associate to form the Fc domain.

In one aspect, the isolated polynucleotide encodes the entire antibody molecule according to the invention as described herein. In another aspect, the isolated polynucleotide encodes a polypeptide (e.g. a heavy chain or a light chain) comprised in the antibody according to the invention as described herein.

In one aspect, the polynucleotide or nucleic acid is DNA. In another aspect, a polynucleotide of the present invention is RNA, for example, in the form of messenger RNA (mRNA). RNA of the present invention may be single stranded or double stranded.

### Recombinant Methods and Compositions

(Bispecific) antibodies may be produced using recombinant methods and compositions. For these methods one or more isolated polynucleotide(s) encoding the antibody are provided.

In one aspect, two polynucleotides are prepared, one for the light chain or a fragment thereof and one for the heavy chain or a fragment thereof Such polynucleotide(s) encode an amino acid sequence comprising the VL and/or an amino acid sequence comprising the VH of the antibody (e.g., the light and/or heavy chain(s) of the antibody). These polynucleotides may be on the same expression vector or on different expression vectors.

In case of a bispecific antibody with heterodimeric heavy chains four polynucleotides are prepared, one for the first light chain, one for the first heavy chain comprising the first heteromonomeric Fc region polypeptide, one for the second light chain, and one for the second heavy chain comprising the second heteromonomeric Fc region polypeptide. The four polynucleotides may be comprised in one or more nucleic acid molecules or expression vectors. Such polynucleotide(s) encode (i) an amino acid sequence comprising the first VL (or, in the case of a crossover Fab molecule being comprised in the bispecific antibody, wherein the VH and VL are replaced by each other: the first VH) and/or (ii) an amino acid sequence comprising the first VH (or, in the case of a crossover Fab molecule being comprised in the bispecific antibody, wherein the VH and VL are replaced by each other: the first VL) including the first heteromonomeric Fc region and/or (iii) an amino acid sequence comprising the second VL and/or (iv) an amino acid sequence comprising the second VH including the second heteromonomeric Fc region of the antibody (e.g., the first and/or second light and/or the first and/or second heavy chains of the antibody). These polynucleotides can be on the same expression vector or on different expression vectors, normally these polynucleotides are located on two or three expression vectors, i.e. one vector can comprise more than one of these polynucleotides. In one aspect, isolated polynucleotides encoding an antibody as used in the methods as described herein are provided.

In one aspect, a method of making an anti-IL-33/anti-IL-6 bispecific antibody of the invention is provided, wherein the method comprises culturing a host cell comprising polynucleotide(s) encoding the antibody, as provided above, under conditions suitable for expression of the antibody, and optionally recovering the antibody from the host cell (including the host cell culture medium).

For recombinant production of a (bispecific) antibody, polynucleotides encoding the antibody, e.g., as described above, are prepared and inserted into one or more vectors for further cloning and/or expression in a host cell. Such polynucleotides may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells as described herein. For example, antibodies may be produced in bacteria, in particular when glycosylation and Fc effector function are not needed. For expression of antibody fragments and polypeptides in bacteria, see, e.g., Charlton, K. A., In: Methods in Molecular Biology, Vol. 248, Lo, B. K. C. (ed.), Humana Press, Totowa, NJ, pp. 245-254 (2003), describing expression of antibody fragments in E. coli. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 1409-1414 (2004) and Li, H. et al., Nat. Biotech. 24: 210-215 (2006).

Suitable host cells for the expression of (glycosylated) antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodopterafrugiperda cells.

Plant cell cultures can also be utilized as hosts. See, e.g., U.S. Pat. Nos. 5,959,177, 6,040,498, 6,420,548, 7,125,978, and 6,417,429 (describing technology for producing antibodies in transgenic plants).

Vertebrate cells may also be used as hosts. For example, mammalian cell lines that are adapted to grow in suspension may be useful. Examples of useful mammalian host cell lines are monkey kidney CV1 line transformed by SV40 (COS-7); human embryonic kidney line (293 or 293T cells); baby hamster kidney cells (BHK); mouse sertoli cells (TM4 cells); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells; MRC 5 cells; and FS4 cells. Other useful mammalian host cell lines include Chinese hamster ovary (CHO) cells, including DHFR-CHO cells; and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ, pp. 255-268 (2004).

In one aspect, the host cell is a eukaryotic cell, e.g., a Chinese Hamster Ovary (CHO) cell or a human embryonic kidney (HEK) cell.

In one aspect, the host cell is an isolated host cell. In one aspect, the host cell is not a cell within a human body.

When recombinant expression vectors encoding antibody genes are introduced into mammalian host cells, the antibodies are produced by culturing the host cells for a period of time sufficient to allow for expression of the antibody in the host cells or, in one aspect, secretion of the antibody into the culture medium in which the host cells are grown. Antibodies can be recovered and purified from recombinant cell cultures by well-known methods including, but not limited to, affinity chromatography (e.g. protein A chromatography), size exclusion chromatography, anion or cation exchange chromatography, mixed-mode chromatography, phosphocellulose chromatography, hydrophobic interaction chromatography, hydroxyapatite chromatography and lectin chromatography.

### Pharmaceutical Compositions

In a further aspect, provided are pharmaceutical compositions comprising the anti-IL-33/anti-IL-6 bispecific antibody of the invention, e.g., for use in any of the therapeutic methods described herein. In one aspect, a pharmaceutical composition comprises the bispecific antibody and a pharmaceutically acceptable carrier. In another aspect, a pharmaceutical composition comprises the bispecific antibody and at least one additional therapeutic agent, e.g., as described below.

Pharmaceutical compositions (formulations) of the anti-IL-33/anti-IL-6 bispecific antibody of the invention can be prepared by combining the antibody with pharmaceutically acceptable carriers or excipients known to the skilled person. See, for example Remington's Pharmaceutical Sciences 16th edition, Osol, A. Ed. (1980) and Falconer R. J., Biotechnology Advances 37: 107412 (2019). Exemplary pharmaceutical compositions of the bispecific antibody may be lyophilized, aqueous, frozen, etc. In one aspect, the pharmaceutical composition provided herein is for intravenous administration. In another aspect, the pharmaceutical composition is for subcutaneous administration.

Pharmaceutically acceptable carriers are generally non-toxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredient as necessary for the particular indication being treated, preferably those with complementary activities that do not adversely affect each other. For example, it may be desirable to further provide active ingredients indicated for the treatment of an inflammatory lung disease, such as COPD. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### Articles of Manufacture

In another aspect of the invention, an article of manufacture containing materials useful for the treatment of an inflammatory lung disease, in particular COPD, is provided. The article of manufacture comprises a container and a label or package insert on or associated with the container.

Suitable containers include, for example, bottles, vials, syringes, IV solution bags, etc. The containers may be formed from a variety of materials such as glass or plastic. The container holds a composition which is by itself or combined with another composition effective for treating the inflammatory lung disease and may have a sterile access port (for example the container may be an intravenous solution bag or a vial having a stopper pierceable by a hypodermic injection needle). At least one active agent in the composition is an anti-IL-33/anti-IL-6 bispecific antibody of the invention. The label or package insert indicates that the composition is used for treating an inflammatory lung disease, in particular COPD. Moreover, the article of manufacture may comprise (a) a first container with a composition contained therein, wherein the composition comprises the bispecific antibody of the invention; and (b) a second container with a composition contained therein, wherein the composition comprises a further therapeutic agent, in particular a further therapeutic agent indicated for the treatment of the inflammatory lung disease. The article of manufacture in this aspect of the invention may further comprise a package insert indicating that the compositions can be used to treat an inflammatory lung disease, in particular COPD. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

AMINO ACID SEQUENCES

| | | SEQ |
|---|---|---|
| | Sequence | ID NO |
| IL-6 | NYLIE | 1 |
| HCDR1 | | |
| IL-6 | VTTPGGGTINYAQKFQG | 2 |
| HCDR2 | | |
| IL-6 | SRWDPLYYYALEY | 3 |
| HCDR3 | | |
| IL-6 VH | QVQLVQSGAEVKKPGSSVKVSCKASGYVLPNYLIEWVRQAP | 4 |
| | GQGLEWMGVTTPGGGTINYAQKFQGRVTITADESTSTAYME | |
| | LSSLRSEDTAVYYCARSRWDPLYYYALEYWGQGTTVTVSS | |
| IL-6 | RASESVDNYGIPFMN | 5 |
| LCDR1 | | |
| IL-6 | AASNRGS | 6 |
| LCDR2 | | |
| IL-6 | QQSEEVPLT | 7 |
| LCDR3 | | |
| IL-6 VL | DIVMTQSPDSLAVSLGERATINCRASESVDNYGIPFMNWYQ | 8 |
| | QKPGQPPKLLIYAASNRGSGVPDRFSGSGSGTDFTLTISSLQA | |
| | EDVAVYYCQQSEEVPLTFGQGTKLEIK | |
| IL-33 | NYWMT | 9 |
| HCDR1 | | |
| IL-33 | SITYTGGGTYYPDSVKG | 10 |
| HCDR2 | | |
| IL-33 | ANYYYNTYGGFPY | 11 |
| HCDR3 | | |
| IL-33 VH | EVQLVESGGGLVQPGGSLRLSCAASGFTFNNYWMTWIRQAP | 12 |
| | GKGLEWVASITYTGGGTYYPDSVKGRFTISRDDAKSSLYLQ | |
| | MNSLRAEDTAVYYCTRANYYYNTYGGFPYWGQGTLVTVSS | |
| IL-33 | LASEGFSNDLA | 13 |
| LCDR1 | | |
| IL-33 | AASRLQD | 14 |
| LCDR2 | | |
| IL-33 | QQGSKYPLT | 15 |
| LCDR3 | | |
| IL-33 VL | DIQMTQSPSSLSASVGDRVTITCLASEGFSNDLAWYQQKPGK | 16 |
| | SPKLLIYAASRLQDGVPSRFSGSGSGTDFTLTISSLQPEDFATY | |
| | FCQQGSKYPLTFGGGTKVEIK | |
| IL-33 LC | DIQMTQSPSSLSASVGDRVTITCLASEGFSNDLAWYQQKPGK | 17 |
| | SPKLLIYAASRLQDGVPSRFSGSGSGTDFTLTISSLQPEDFATY | |
| | FCQQGSKYPLTFGGGTKVEIKRTVAAPSVFIFPPSDRKLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| IL-33 HC | EVQLVESGGGLVQPGGSLRLSCAASGFTFNNYWMTWIRQAP | 18 |
| (knob, | GKGLEWVASITYTGGGTYYPDSVKGRFTISRDDAKSSLYLQ | |
| PGLALA) | MNSLRAEDTAVYYCTRANYYYNTYGGFPYWGQGTLVTVSS | |
| | ASTKGPSVFPLAPSSKSTSGGTAALGCLVEDYFPEPVTVSWN | |
| | SGALTSGVHTFPAVLQSSGLYSLSSVVTVPSSSLGTQTYICNV | |
| | NHKPSNTKVDEKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPCRDELTKNQVSLWC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| IL-6 | DIVMTQSPDSLAVSLGERATINCRASESVDNYGIPFMNWYQ | 19 |
| (Crossfab) | QKPGQPPKLLIYAASNRGSGVPDRFSGSGSGTDFTLTISSLQA | |
| HC (hole, | EDVAVYYCQQSEEVPLTFGQGTKLEIKSSASTKGPSVFPLAP | |
| PGLALA) | SSKSTSGGTAALGCLVKDYFPEPVTVSWNSGALTSGVHTFP | |
| | AVLQSSGLYSLSSVVTVPSSSLGTQTYICNVNHKPSNTKVDK | |
| | KVEPKSCDKTHTCPPCPAPEAAGGPSVFLFPPKPKDTLMISRT | |
| | PEVTCVVVDVSHEDPEVKFNWYVDGVEVHNAKTKPREEQY | |
| | NSTYRVVSVLTVLHQDWLNGKEYKCKVSNKALGAPIEKTIS | |
| | KAKGQPREPQVCTLPPSRDELTKNQVSLSCAVKGFYPSDIAV | |
| | EWESNGQPENNYKTTPPVLDSDGSFFLVSKLTVDKSRWQQG | |
| | NVFSCSVMHEALHNHYTQKSLSLSPG | |
| IL-6 | QVQLVQSGAEVKKPGSSVKVSCKASGYVLPNYLIEWVRQAP | 20 |
| (Crossfab) | GQGLEWMGVTTPGGGTINYAQKFQGRVTITADESTSTAYME | |
| LC | LSSLRSEDTAVYYCARSRWDPLYYYALEYWGQGTTVTVSS | |
| | ASVAAPSVFIFPPSDEQLKSGTASVVCLLNNFYPREAKVQWK | |
| | VDNALQSGNSQESVTEQDSKDSTYSLSSTLTLSKADYEKHK | |
| | VYACEVTHQGLSSPVTKSFNRGEC | |
| IL-33 HC | EVQLVESGGGLVQPGGSLRLSCAASGFTFNNYWMTWIRQAP | 21 |
| (knob, | GKGLEWVASITYTGGGTYYPDSVKGRFTISRDDAKSSLYLQ | |
| PGLALA)- | MNSLRAEDTAVYYCTRANYYYNTYGGFPYWGQGTLVTVSS | |
| IL-6 | ASTKGPSVFPLAPSSKSTSGGTAALGCLVEDYFPEPVTVSWN | |
| (Crossfab) | SGALTSGVHTFPAVLQSSGLYSLSSVVTVPSSSLGTQTYICNV | |
| HC | NHKPSNTKVDEKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPCRDELTKNQVSLWC | |
| | LVKGFYPSDIAVEWESNGQPENNYKTTPPVLDSDGSFFLYSK | |
| | LTVDKSRWQQGNVFSCSVMHEALHNHYTQKSLSLSPGGGS | |
| | GGGGSGGGGSGGGGSGGDIVMTQSPDSLAVSLGERATINCR | |
| | ASESVDNYGIPFMNWYQQKPGQPPKLLIYAASNRGSGVPDR | |
| | FSGSGSGTDFTLTISSLQAEDVAVYYCQQSEEVPLTFGQGTK | |
| | LEIKSSASTKGPSVFPLAPSSKSTSGGTAALGCLVKDYFPEPV | |
| | TVSWNSGALTSGVHTFPAVLQSSGLYSLSSVVTVPSSSLGTQ | |
| | TYICNVNHKPSNTKVDKKVEPKSCD | |
| IL-33 HC | EVQLVESGGGLVQPGGSLRLSCAASGFTFNNYWMTWIRQAP | 22 |
| (hole, | GKGLEWVASITYTGGGTYYPDSVKGRFTISRDDAKSSLYLQ | |
| PGLALA) | MNSLRAEDTAVYYCTRANYYYNTYGGFPYWGQGTLVTVSS | |
| | ASTKGPSVFPLAPSSKSTSGGTAALGCLVEDYFPEPVTVSWN | |
| | SGALTSGVHTFPAVLQSSGLYSLSSVVTVPSSSLGTQTYICNV | |
| | NHKPSNTKVDEKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVCTLPPSRDELTKNQVSLSCA | |
| | VKGFYPSDIAVEWESNGQPENNYKTTPPVLDSDGSFFLVSKL | |
| | TVDKSRWQQGNVFSCSVMHEALHNHYTQKSLSLSPG | |
| IL-33 HC | EVQLVESGGGLVQPGGSLRLSCAASGFTFNNYWMTWIRQAP | 23 |
| (knob, | GKGLEWVASITYTGGGTYYPDSVKGRFTISRDDAKSSLYLQ | |
| PGLALA, | MNSLRAEDTAVYYCTRANYYYNTYGGFPYWGQGTLVTVSS | |
| YTE) | ASTKGPSVFPLAPSSKSTSGGTAALGCLVEDYFPEPVTVSWN | |
| | SGALTSGVHTFPAVLQSSGLYSLSSVVTVPSSSLGTQTYICNV | |
| | NHKPSNTKVDEKVEPKSCDKTHTCPPCPAPEAAGGPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPCRDELTKNQVSLWC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| IL-6 | DIVMTQSPDSLAVSLGERATINCRASESVDNYGIPFMNWYQ | 24 |
| (Crossfab) | QKPGQPPKLLIYAASNRGSGVPDRFSGSGSGTDFTLTISSLQA | |
| HC (hole, | EDVAVYYCQQSEEVPLTFGQGTKLEIKSSASTKGPSVFPLAP | |
| PGLALA, | SSKSTSGGTAALGCLVKDYFPEPVTVSWNSGALTSGVHTFP | |
| YTE) | AVLQSSGLYSLSSVVTVPSSSLGTQTYICNVNHKPSNTKVDK | |
| | KVEPKSCDKTHTCPPCPAPEAAGGPSVFLFPPKPKDTLYITRE | |
| | PEVTCVVVDVSHEDPEVKFNWYVDGVEVHNAKTKPREEQY | |
| | NSTYRVVSVLTVLHQDWLNGKEYKCKVSNKALGAPIEKTIS | |
| | KAKGQPREPQVCTLPPSRDELTKNQVSLSCAVKGFYPSDIAV | |
| | EWESNGQPENNYKTTPPVLDSDGSFFLVSKLTVDKSRWQQG | |
| | NVFSCSVMHEALHNHYTQKSLSLSPG | |
| IL-33 HC | EVQLVESGGGLVQPGGSLRLSCAASGFTFNNYWMTWIRQAP | 25 |
| (knob, | GKGLEWVASITYTGGGTYYPDSVKGRFTISRDDAKSSLYLQ | |
| PGLALA, | MNSLRAEDTAVYYCTRANYYYNTYGGFPYWGQGTLVTVSS | |
| LS) | ASTKGPSVFPLAPSSKSTSGGTAALGCLVEDYFPEPVTVSWN | |
| | SGALTSGVHTFPAVLQSSGLYSLSSVVTVPSSSLGTQTYICNV | |
| | NHKPSNTKVDEKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPCRDELTKNQVSLWC | |
| | LVKGFYPSDIAVEWESNGQPENNYKTTPPVLDSDGSFFLYSK | |
| | LTVDKSRWQQGNVFSCSVLHEALHSHYTQKSLSLSPG | |
| IL-6 | DIVMTQSPDSLAVSLGERATINCRASESVDNYGIPFMNWYQ | 26 |
| (Crossfab) | QKPGQPPKLLIYAASNRGSGVPDRFSGSGSGTDFTLTISSLQA | |
| HC (hole, | EDVAVYYCQQSEEVPLTFGQGTKLEIKSSASTKGPSVFPLAP | |
| PGLALA, | SSKSTSGGTAALGCLVKDYFPEPVTVSWNSGALTSGVHTFP | |
| LS) | AVLQSSGLYSLSSVVTVPSSSLGTQTYICNVNHKPSNTKVDK | |
| | KVEPKSCDKTHTCPPCPAPEAAGGPSVFLFPPKPKDTLMISRT | |
| | PEVTCVVVDVSHEDPEVKFNWYVDGVEVHNAKTKPREEQY | |
| | NSTYRVVSVLTVLHQDWLNGKEYKCKVSNKALGAPIEKTIS | |
| | KAKGQPREPQVCTLPPSRDELTKNQVSLSCAVKGFYPSDIAV | |
| | EWESNGQPENNYKTTPPVLDSDGSFFLVSKLTVDKSRWQQG | |
| | NVFSCSVLHEALHSHYTQKSLSLSPG | |
| IL-33 | EVQLVESGGGLVQPGGSLRLSCAASGFTFNNYWMTWIRQAP | 27 |
| (Crossfab) | GKGLEWVASITYTGGGTYYPDSVKGRFTISRDDAKSSLYLQ | |
| LC | MNSLRAEDTAVYYCTRANYYYNTYGGFPYWGQGTLVTVSS | |
| | ASVAAPSVFIFPPSDEQLKSGTASVVCLLNNFYPREAKVQWK | |
| | VDNALQSGNSQESVTEQDSKDSTYSLSSTLTLSKADYEKHK | |
| | VYACEVTHQGLSSPVTKSFNRGEC | |
| IL-33 | DIQMTQSPSSLSASVGDRVTITCLASEGFSNDLAWYQQKPGK | 28 |
| (Crossfab) | SPKLLIYAASRLQDGVPSRFSGSGSGTDFTLTISSLQPEDFATY | |
| HC (knob, | FCQQGSKYPLTFGGGTKVEIKSSASTKGPSVFPLAPSSKSTSG | |
| PGLALA) | GTAALGCLVKDYFPEPVTVSWNSGALTSGVHTFPAVLQSSG | |
| | LYSLSSVVTVPSSSLGTQTYICNVNHKPSNTKVDKKVEPKSC | |
| | DKTHTCPPCPAPEAAGGPSVFLFPPKPKDTLMISRTPEVTCV | |
| | VVDVSHEDPEVKFNWYVDGVEVHNAKTKPREEQYNSTYRV | |
| | VSVLTVLHQDWLNGKEYKCKVSNKALGAPIEKTISKAKGQP | |
| | REPQVYTLPPCRDELTKNQVSLWCLVKGFYPSDIAVEWESN | |
| | GQPENNYKTTPPVLDSDGSFFLYSKLTVDKSRWQQGNVFSC | |
| | SVMHEALHNHYTQKSLSLSPG | |
| IL-6 HC | QVQLVQSGAEVKKPGSSVKVSCKASGYVLPNYLIEWVRQAP | 29 |
| (hole, | GQGLEWMGVTTPGGGTINYAQKFQGRVTITADESTSTAYME | |
| PGLALA) | LSSLRSEDTAVYYCARSRWDPLYYYALEYWGQGTTVTVSS | |
| | ASTKGPSVFPLAPSSKSTSGGTAALGCLVEDYFPEPVTVSWN | |
| | SGALTSGVHTFPAVLQSSGLYSLSSVVTVPSSSLGTQTYICNV | |
| | NHKPSNTKVDEKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVCTLPPSRDELTKNQVSLSCA | |
| | VKGFYPSDIAVEWESNGQPENNYKTTPPVLDSDGSFFLVSKL | |
| | TVDKSRWQQGNVFSCSVMHEALHNHYTQKSLSLSPG | |
| IL-6 LC | DIVMTQSPDSLAVSLGERATINCRASESVDNYGIPFMNWYQ | 30 |
| | QKPGQPPKLLIYAASNRGSGVPDRFSGSGSGTDFTLTISSLQA | |
| | EDVAVYYCQQSEEVPLTFGQGTKLEIKRTVAAPSVFIFPPSDR | |
| | KLKSGTASVVCLLNNFYPREAKVQWKVDNALQSGNSQESV | |
| | TEQDSKDSTYSLSSTLTLSKADYEKHKVYACEVTHQGLSSPV | |
| | TKSFNRGEC | |
| IL-33 | DIQMTQSPSSLSASVGDRVTITCLASEGFSNDLAWYQQKPGK | 31 |
| (Crossfab) | SPKLLIYAASRLQDGVPSRFSGSGSGTDFTLTISSLQPEDFATY | |
| HC (knob, | FCQQGSKYPLTFGGGTKVEIKSSASTKGPSVFPLAPSSKSTSG | |
| PGLALA, | GTAALGCLVKDYFPEPVTVSWNSGALTSGVHTFPAVLQSSG | |
| YTE) | LYSLSSVVTVPSSSLGTQTYICNVNHKPSNTKVDKKVEPKSC | |
| | DKTHTCPPCPAPEAAGGPSVFLFPPKPKDTLYITREPEVTCVV | |
| | VDVSHEDPEVKFNWYVDGVEVHNAKTKPREEQYNSTYRVV | |
| | SVLTVLHQDWLNGKEYKCKVSNKALGAPIEKTISKAKGQPR | |
| | EPQVYTLPPCRDELTKNQVSLWCLVKGFYPSDIAVEWESNG | |
| | QPENNYKTTPPVLDSDGSFFLYSKLTVDKSRWQQGNVFSCS | |
| | VMHEALHNHYTQKSLSLSPG | |
| IL-6 HC | QVQLVQSGAEVKKPGSSVKVSCKASGYVLPNYLIEWVRQAP | 32 |
| (hole, | GQGLEWMGVTTPGGGTINYAQKFQGRVTITADESTSTAYME | |
| PGLALA, | LSSLRSEDTAVYYCARSRWDPLYYYALEYWGQGTTVTVSS | |
| YTE) | ASTKGPSVFPLAPSSKSTSGGTAALGCLVEDYFPEPVTVSWN | |
| | SGALTSGVHTFPAVLQSSGLYSLSSVVTVPSSSLGTQTYICNV | |
| | NHKPSNTKVDEKVEPKSCDKTHTCPPCPAPEAAGGPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVCTLPPSRDELTKNQVSLSCA | |
| | VKGFYPSDIAVEWESNGQPENNYKTTPPVLDSDGSFFLVSKL | |
| | TVDKSRWQQGNVFSCSVMHEALHNHYTQKSLSLSPG | |
| IL-33 HC- | EVQLVESGGGLVQPGGSLRLSCAASGFTFNNYWMTWIRQAP | 33 |
| IL-6 | GKGLEWVASITYTGGGTYYPDSVKGRFTISRDDAKSSLYLQ | |
| (Crossfab) | MNSLRAEDTAVYYCTRANYYYNTYGGFPYWGQGTLVTVSS | |
| HC (knob, | ASTKGPSVFPLAPSSKSTSGGTAALGCLVEDYFPEPVTVSWN | |
| PGLALA) | SGALTSGVHTFPAVLQSSGLYSLSSVVTVPSSSLGTQTYICNV | |
| | NHKPSNTKVDEKVEPKSCGGGGSGGGGDIVMTQSPDSLAVS | |
| | LGERATINCRASESVDNYGIPFMNWYQQKPGQPPKLLIYAAS | |
| | NRGSGVPDRFSGSGSGTDFTLTISSLQAEDVAVYYCQQSEEV | |
| | PLTFGQGTKLEIKSSASTKGPSVFPLAPSSKSTSGGTAALGCL | |
| | VKDYFPEPVTVSWNSGALTSGVHTFPAVLQSSGLYSLSSVVT | |
| | VPSSSLGTQTYICNVNHKPSNTKVDKKVEPKSCDKTHTCPPC | |
| | PAPEAAGGPSVFLFPPKPKDTLMISRTPEVTCVVVDVSHEDP | |
| | EVKFNWYVDGVEVHNAKTKPREEQYNSTYRVVSVLTVLHQ | |
| | DWLNGKEYKCKVSNKALGAPIEKTISKAKGQPREPQVYTLP | |
| | PCRDELTKNQVSLWCLVKGFYPSDIAVEWESNGQPENNYKT | |
| | TPPVLDSDGSFFLYSKLTVDKSRWQQGNVFSCSVMHEALHN | |
| | HYTQKSLSLSPG | |
| Fc (knob, | DKTHTCPPCPAPEAAGGPSVFLFPPKPKDTLMISRTPEVTCV | 34 |
| PGLALA)- | VVDVSHEDPEVKFNWYVDGVEVHNAKTKPREEQYNSTYRV | |
| IL-33 | VSVLTVLHQDWLNGKEYKCKVSNKALGAPIEKTISKAKGQP | |
| HC | REPQVYTLPPCRDELTKNQVSLWCLVKGFYPSDIAVEWESN | |
| | GQPENNYKTTPPVLDSDGSFFLYSKLTVDKSRWQQGNVFSC | |
| | SVMHEALHNHYTQKSLSLSPGGGSGGGGSGGGGSGGEVQL | |
| | VESGGGLVQPGGSLRLSCAASGFTFNNYWMTWIRQAPGKG | |
| | LEWVASITYTGGGTYYPDSVKGRFTISRDDAKSSLYLQMNS | |
| | LRAEDTAVYYCTRANYYYNTYGGFPYWGQGTLVTVSSAST | |
| | KGPSVFPLAPSSKSTSGGTAALGCLVEDYFPEPVTVSWNSGA | |
| | LTSGVHTFPAVLQSSGLYSLSSVVTVPSSSLGTQTYICNVNH | |
| | KPSNTKVDEKVEPKSCD | |
| Fc (hole, | DKTHTCPPCPAPEAAGGPSVFLFPPKPKDTLMISRTPEVTCV | 35 |
| PGLALA)- | VVDVSHEDPEVKFNWYVDGVEVHNAKTKPREEQYNSTYRV | |
| IL-6 | VSVLTVLHQDWLNGKEYKCKVSNKALGAPIEKTISKAKGQP | |
| (CH1/CL | REPQVCTLPPSRDELTKNQVSLSCAVKGFYPSDIAVEWESNG | |
| Crossfab) | QPENNYKTTPPVLDSDGSFFLVSKLTVDKSRWQQGNVFSCS | |
| HC | VMHEALHNHYTQKSLSLSPGGGSGGGGSGGGGSGGQVQLV | |
| | QSGAEVKKPGSSVKVSCKASGYVLPNYLIEWVRQAPGQGLE | |
| | WMGVTTPGGGTINYAQKFQGRVTITADESTSTAYMELSSLR | |
| | SEDTAVYYCARSRWDPLYYYALEYWGQGTTVTVSSASVAA | |
| | PSVFIFPPSDEQLKSGTASVVCLLNNFYPREAKVQWKVDNAL | |
| | QSGNSQESVTEQDSKDSTYSLSSTLTLSKADYEKHKVYACE | |
| | VTHQGLSSPVTKSFNRGEC | |
| IL-6 | DIVMTQSPDSLAVSLGERATINCRASESVDNYGIPFMNWYQ | 36 |
| (CH1/CL | QKPGQPPKLLIYAASNRGSGVPDRFSGSGSGTDFTLTISSLQA | |
| Crossfab) | EDVAVYYCQQSEEVPLTFGQGTKLEIKSSASTKGPSVFPLAP | |
| LC | SSKSTSGGTAALGCLVKDYFPEPVTVSWNSGALTSGVHTFP | |
| | AVLQSSGLYSLSSVVTVPSSSLGTQTYICNVNHKPSNTKVDK | |
| | KVEPKSCD | |
| Human | VPPGEDSKDVAAPHRQPLTSSERIDKQIRYILDGISALRKETC | 37 |
| IL-6 | NKSNMCESSKEALAENNLNLPKMAEKDGCFQSGFNEETCLV | |
| | KIITGLLEFEVYLEYLQNRFESSEEQARAVQMSTKVLIQFLQK | |
| | KAKNLDAITTPDPTTNASLLTKLQAQNQWLQDMTTHLILRS | |
| | FKEFLQSSLRALRQM | |
| Human | MKPKMKYSTNKISTAKWKNTASKALCFKLGKSQQKAKEVC | 38 |
| IL-33 | PMYFMKLRSGLMIKKEACYFRRETTKRPSLKTGRKHKRHLV | |
| | LAACQQQSTVECFAFGISGVQKYTRALHDSSITGISPITEYLA | |
| | SLSTYNDQSITFALEDESYEIYVEDLKKDEKKDKVLLSYYES | |
| | QHPSNESGDGVDGKMLMVTLSPTKDFWLHANNKEHSVELH | |
| | KCEKPLPDQAFFVLHNMHSNCVSFECKTDPGVFIGVKDNHL | |
| | ALIKVDSSENLCTENILFKLSET | |
| hlgG1 Fc | DKTHTCPPCPAPELLGGPSVFLFPPKPKDTLMISRTPEVTCVV | 39 |
| region | VDVSHEDPEVKFNWYVDGVEVHNAKTKPREEQYNSTYRVV | |
| | SVLTVLHQDWLNGKEYKCKVSNKALPAPIEKTISKAKGQPR | |
| | EPQVYTLPPSRDELTKNQVSLTCLVKGFYPSDIAVEWESNGQ | |
| | PENNYKTTPPVLDSDGSFFLYSKLTVDKSRWQQGNVFSCSV | |
| | MHEALHNHYTQKSLSLSPG | |
| Human | RTVAAPSVFIFPPSDEQLKSGTASVVCLLNNFYPREAKVQWK | 40 |
| kappa CL | VDNALQSGNSQESVTEQDSKDSTYSLSSTLTLSKADYEKHK | |
| domain | VYACEVTHQGLSSPVTKSFNRGEC | |
| Human | QPKAAPSVTLFPPSSEELQANKATLVCLISDFYPGAVTVAWK | 41 |
| lambda | ADSSPVKAGVETTTPSKQSNNKYAASSYLSLTPEQWKSHRS | |
| CL | YSCQVTHEGSTVEKTVAPTECS | |
| domain | | |
| Human | ASTKGPSVFPLAPSSKSTSGGTAALGCLVKDYFPEPVTVSWN | 42 |
| IgG1 | SGALTSGVHTFPAVLQSSGLYSLSSVVTVPSSSLGTQTYICNV | |
| heavy | NHKPSNTKVDKKVEPKSCDKTHTCPPCPAPELLGGPSVFLFP | |
| chain | PKPKDTLMISRTPEVTCVVVDVSHEDPEVKFNWYVDGVEVH | |
| constant | NAKTKPREEQYNSTYRVVSVLTVLHQDWLNGKEYKCKVSN | |
| region | KALPAPIEKTISKAKGQPREPQVYTLPPSRDELTKNQVSLTCL | |
| (CH1- | VKGFYPSDIAVEWESNGQPENNYKTTPPVLDSDGSFFLYSKL | |
| CH2-CH3) | TVDKSRWQQGNVFSCSVMHEALHNHYTQKSLSLSP | |
| IL-33 VH | EVQLVESGGNLEQPGGSLRLSCTASGFTFSRSAMNWVRRAP | 43 |
| (itepeki- | GKGLEWVSGISGSGGRTYYADSVKGRFTISRDNSKNTLYLQ | |
| mab) | MNSLSAEDTAAYYCAKDSYTTSWYGGMDVWGHGTTVTVSS | |
| IL-33 VL | DIQMTQSPSSVSASVGDRVTITCRASQGIFSWLAWYQQKPG | 44 |
| (itepeki- | KAPKLLIYAASSLQSGVPSRFSGSGSGTDFTLTISSLQPEDFAI | |
| mab) | YYCQQANSVPITFGQGTRLEIK | |

### EXAMPLES

The following are examples of methods and compositions of the invention. It is understood that various other aspects may be practiced, given the general description provided above.

### Example 1—ICAM-1 Expression on the Surface of the Endothelial Cells

The lung endothelium plays a pivotal role in maintaining proper lung function both during homeostasis and in response to inflammatory stimuli. It is essential for maintaining homeostasis by regulating barrier function, vascular tone, and anti-inflammatory properties. During inflammatory responses, it becomes activated to facilitate immune cell recruitment and vascular permeability, playing a crucial role in both initiating and resolving inflammation.

Endothelial inflammation is a significant factor in the pathogenesis and progression of Chronic Obstructive Pulmonary Disease (COPD). The endothelium in COPD patients often exhibits increased expression of adhesion molecules such as ICAM-1 and VCAM-1, which facilitate the recruitment and transmigration of inflammatory cells like neutrophils and macrophages into the lung tissue. This persistent influx of immune cells contributes to chronic inflammation, tissue damage, and remodeling of the airways and alveoli, hallmark features of COPD (Hogg et al., 2004). It has been shown that COPD patients have significantly impaired endothelial function compared to healthy individuals. In addition, evidence suggests that biomarkers of endothelial dysfunction can predict COPD exacerbations and disease progression.

Several inflammatory cytokines contribute to vascular inflammation and have been implicated in the pathogenesis of COPD.

As shown in the present Examples, we found that IL-33 and IL-6 play independent roles in mediating endothelial dysfunction and cooperatively increase endothelial ICAM-1 expression and endothelial immune cell transmigration. Importantly, we also found that inhibition of both cytokines is required to fully prevent endothelial cell activation.

### Material and Methods

Primary Human Umbilical Vein Endothelial Cells (HUVEC) from pooled donors were obtained from LONZA (Cat #00191027, LOT #18TL232828, 22TL115625 and 9F3043) and expanded in EGM™-2 Endothelial Cell Growth Medium (Lonza, Cat #CC-3162) supplemented with EGM-2 Bullet kit in T150 cell culture flasks coated with Attachment Factor Protein (Gibco, Cat #S006100) until 80% confluence. Cells were detached using STEMPRO™ ACCUTASE™ Cell Dissociation Reagent (Gibco, Cat #A1110501) and seeded on non-coated 96-well plates (Corning, Cat #3595) at a density of 30.000 cells/well and allowed to attach overnight in a humidified incubator (37° C., 5% CO₂). Cells were stimulated with IL33 (R&D Systems, Cat #3625-IL-010/CF), IL6, and IL6R in equimolar amounts (R&D Systems, Cat #206-IL/CF and Cat #227-SR-025) or the combination of IL33 and IL6 and IL6R at concentrations ranging from 300 ng/mL to 0.4 ng/mL. Cytokines were prepared as 20× concentrated solutions and added to the cells alongside fresh medium. After 48 h, cells from each well were washed with DPBS, detached with STEMPRO™ ACCUTASE™ Cell Dissociation Reagent, and transferred to a U-bottom 96-well plate (Corning, Cat #3799). Cells were washed with BD PHARMINGEN™ Stain Buffer (BD, Cat #554656) containing 1 mM EDTA by centrifugation and stained with Human ICAM-1/CD54 Fluorescein-conjugated Antibody (R&D Systems, Cat #BBA20) for 20 minutes at 4° C. protected from light. After two washes, cells were acquired on a Beckman Coulter Cytoflex LX system. Cells were identified by first gating on live cells (FSC-A vs. SSC-A) and then on singlets (FSC-A versus FSC-H) followed by gating on ICAM-1+ cells where ICAM-1 levels in non-stimulated cells were defined as baseline expression levels. Graphs show combined data of n=3 cell lots.

Experiments that included testing of antibodies (Example 4) required an additional incubation period before addition to the cells, where 20× concentrated solutions of antibodies were combined with 20× concentrated solutions of cytokines and allowed to stand at room temperature for 45 minutes.

### Results

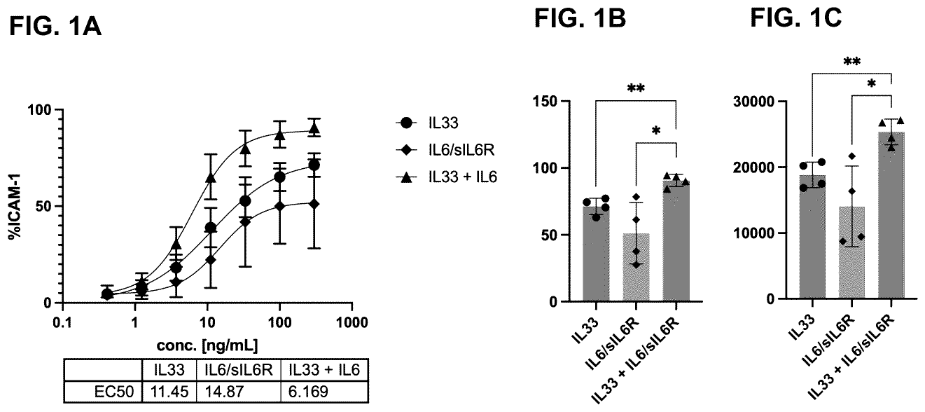

HUVEC cells were cultured in 2D and treated with increasing concentrations of IL-33, the complex of IL-6 and soluble IL-6 receptor (IL6/sIL6R), or the combination of the two cytokines in equimolar amounts. We used FACS to detect the levels of ICAM-1 expression on the surface of the endothelial cells. The amount of each cytokine required to achieve ICAM-1 expression of 50% of the cells (EC50) was 1.8-fold higher for IL-33 and 2.4-fold higher for IL-6/sIL-6R (FIG. 1A). The combination of IL-33 and IL-6/sIL-6R increased the maximum percentage of cells that expressed ICAM-1 levels compared to either cytokine alone (FIG. 1A, FIG. 1B). This resulted in a statistically significant increase in the area under the curve (AUC) indicating that the combination of IL-33 and IL-6/sIL-6R has a significantly higher impact on ICAM-1 upregulation than either cytokine alone (FIG. 1C).

### Example 2—Immune Cell Migration Through a HUVEC Vascular Tube

### Materials and Methods

### 1. Endothelial Tubule Formation

Primary Human Umbilical Vein Endothelial Cells (HUVEC) from pooled donors were obtained from LONZA (Cat #00191027, LOT #18TL232828) and expanded in EGM™-2 Endothelial Cell Growth Medium (Lonza, Cat #CC-3162) supplemented with EGM-2 Bullet kit in T150 cell culture flasks coated with Attachment Factor Protein (Gibco, Cat #S006100) until 80% confluence. Cells were detached using STEMPRO™ ACCUTASE™ Cell Dissociation Reagent (Gibco, Cat #A1110501).

MIMETAS ORGANOREADY® Collagen 3-lane 64 plates (MIMETAS, Cat #MI-OR-CO-CU-02 were used for this study. HUVECs were detached from the flask, resuspended in endothelial medium, counted using Trypan Blue exclusion, and resuspended to a cell seeding density of 15×10⁶ cells/mL. To each right bottom port, 50 μL of medium was added prior to dispensing 2 μL of cell suspension into the right top inlet port using the automatic repeater pipette. The cell suspension was regularly mixed in order to ensure homogenous cell seeding density. The OrganoPlates were placed with the lid, forming a 75-degree angle against the plate stand, and left in this orientation for 5 h to allow cells to attach. After cell attachment, 50 μL of the endothelial medium was added into the top medium inlet. The plate was then placed on the ORGANOFLOW® in a humidified incubator set to an inclination of 14° and an interval of 8 minutes for 48 h to allow for tubule formation.

### B. Activation of PBMCs

Human peripheral blood mononuclear cells (PBMCs) are isolated from Buffy Coats obtained from the Regional Blood Donation Center Luzern by density gradient centrifugation and cryopreserved in Cryostor CS10 (Sigma, C2874) in a rate-controlled freezer over the course of one hour and transferred to LN2 storage until further use. PBMCs were activated in AIM V medium (Gibco, Cat #12055091) containing 20 IU/mL of IL2 (Miltenyi, Cat #130-097-743) and Human TransAct (Miltenyi, Cat #130-111-160) at 1:500 for 72 h followed by a 48 h rest period in fresh medium.

### C. Transmigration of Immune Cells Through the Endothelial Cell Layer

PBMCs were carefully harvested and centrifuged at 300×g for 5 min, counted, and incubated with 2.5 μM Cell Tracker Orange CMRA (Gibco, Cat #C34551) in AIM V medium at a cell concentration of 10E⁶ cells/mL for 30 min at 37° C. Tubes were wrapped in foil and inverted several times to gently mix. T-cells were then centrifuged and pelleted to wash out the stain and resuspended in assay medium (AIM V medium containing 20 IU/mL IL-2, 5 ng/mL VEGF (Peprotech, Cat #100-20) and 5 ng/mL bFGF (Peprotech, Cat #100-18B)) with the final density of 1×10⁶ cells/mL.

At this stage, the medium was carefully aspirated from the right top and bottom inlets of the ORGANOREADY® Plate and 50 μL of immune cell suspension was added to the right top inlet followed by addition of 100 ng/mL IL33 (AdipoGen Life Sciences, Cat #AG-40B-0160-C100) or 100 ng/mL IL6 and 200 ng/mL IL6R (R&D Systems, Cat #206-IL/CF and Cat #227-SR-025) or a combination of all three in assay medium into the right bottom inlet (all in 50 μL). Finally, 50 μL of 40 nM CXCL12 in assay medium was added to the left top and bottom inlet ports as a chemoattractant trigger. The plate was then placed on the ORGANOFLOW® in a humidified incubator set to an inclination of 140 and an interval of 8 minutes for 48 h to allow for immune cell migration. Images were acquired on an Opera Phenix System with a 10× objective and analyzed using PerkinElmer's Harmony software.

### Results

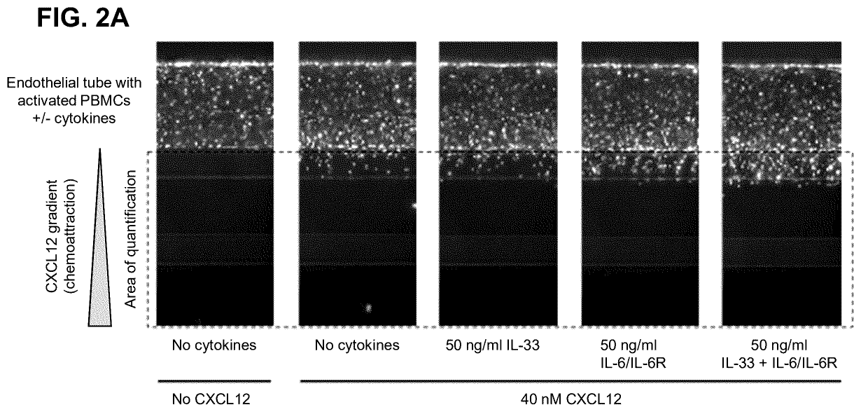

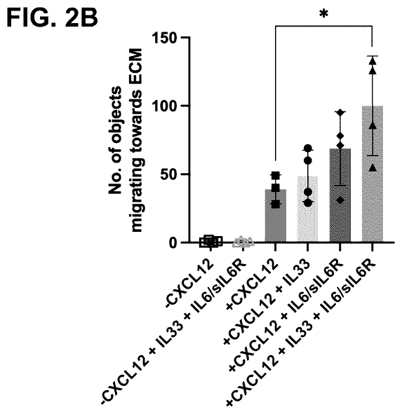

Given the importance of ICAM-1 in immune cell interactions with the endothelium, we investigated the effects of stimulation with IL-33 and IL-6/sIL-6R on the transmigration of human peripheral blood mononuclear cells (PBMCs) through an endothelial layer. We utilized a microfluidic chip consisting of two independently accessible microfluidic channels on either side of an ECM scaffold. Endothelial cells were seeded in one of the channels and once the endothelial cells had grown to form a tubule in the microfluidic channel and against the ECM gel, PBMCs previously activated with (CD3/CD28) beads were added to the lumen of the endothelial tubule. We assessed their migration through the endothelial layer and into the ECM towards a chemoattractant gradient of CXCL12 as has been described before (de Haan et al., 2021). Fluorescently labeled, activated PBMCs transmigrate through the endothelium and into the ECM towards the gradient of CXCL12. Treatment with either cytokine increased the transmigration of activated PBMCs and the combination of treatment with IL-33 and IL-6/sIL-6R resulted in a statistically significant increased transmigration (FIG. 2A, FIG. 2B).

### Example 3—Anti-IL-33/Anti-IL-6 Bispecific Antibodies

The fact that IL-33 and IL-6/sIL-6R act cooperatively to induce endothelial dysfunction that can lead to greater immune cell infiltration in the context of inflammatory responses, suggests that simultaneously blocking both cytokines will be beneficial for patients with chronic inflammatory diseases such as COPD. However, recent clinical trials suggest that high levels of blocking antibodies blocking IL-33 or its receptor ST2 are required to fully inhibit the pathway and provide benefit to patients. As such, a combination approach of two therapeutic antibodies dosed at the same time would be a significant treatment burden as multiple subcutaneous dosing injections would be required to co-deliver sufficient doses of IL-33 and IL-6 blocking antibodies. For this reason, we aimed at developing novel molecules that block both cytokines at once and are sufficiently potent to be dosed as a single molecule.

Based on previously described blocking antibodies against IL-33 (1E1v8; see PCT publication no. WO 2021/183849) and against IL-6 (vamikibart; see WHO Drug Information, vol. 37, no. 3, 2023 (Recommended INN List 90), p. 881), we generated a series of bispecific molecules to determine the optimal configuration for the anti-IL-33/anti-IL-6 bispecific antibody. All molecules comprise the CDR and VH sequences as summarized in Table 1. An overview of all molecules generated in

TABLE 1
CDR and VH sequences of bispecific molecules generated in this Example.

| | HCDR1 | HCDR2 | HCDR3 | VH | LCDR1 | LCDR2 | LCDR3 | VL |
|---|---|---|---|---|---|---|---|---|
| IL-6 | 1 | 2 | 3 | 4 | 5 | 6 | 7 | 8 |
| IL-33 | 9 | 10 | 11 | 12 | 13 | 14 | 15 | 16 |

TABLE 2
Overview of bispecific molecules generated in this Example.

| Molecule ID | Format* | Crossover | Fc mutation | SEQ ID NOs⁺ | FIGS. |
|---|---|---|---|---|---|
| P1AK9539 | 1 + 1, IgG-like | IL-6 | KiH, PGLALA | 17, 18, 19, 20 | 3A-3E |
| | | (VH/VL) | | | |
| P1AM2967 | 2 + 1, IgG-like | IL-6 | KiH, PGLALA | 17, 20, 21, 22 | 7A-7E |
| | with C- | (VH/VL) | | | |
| | terminal IL-6 | | | | |
| P1AM2965 | 1 + 1, IgG-like | IL-6 | KiH, PGLALA, | 17, 20, 23, 24 | 3A-3E |
| | | (VH/VL) | YTE | | |
| P1AM2966 | 1 + 1, IgG-like | IL-6 | KiH, PGLALA, | 17, 20, 25, 26 | 3A-3E |
| | | (VH/VL) | LS | | |
| P1AM3782 | 1 + 1, IgG-like | IL-33 | KiH, PGLALA | 27, 28, 29, 30 | 4A-4E |
| | | (VH/VL) | | | |
| P1AM3779 | 1 + 1, IgG-like | IL-33 | KiH, PGLALA, | 27, 30, 31, 32 | 4A-4E |
| | | (VH/VL) | YTE | | |
| P1AM3781 | 2 + 1, IgG-like | IL-6 | KiH, PGLALA | 17, 20, 22, 33 | 6A-6E |
| | with N- | (VH/VL) | | | |
| | terminal IL- | | | | |
| | 33 | | | | |
| P1AM3780 | 1 + 1, | IL-6 | KiH, PGLALA | 17, 34, 35, 36 | 5A-5E |
| | “inverted” | (CH1/CL) | | | |
| | IgG-like | | | | |

*“1 + 1” refers to formats with one IL-6 binding domain and one IL-33 binding domain; “2 + 1” refers to formats with one IL-6 binding domain and two IL-33 binding domains.
⁺Full heavy and light chain sequences.

### Recombinant Production of Bispecific Antibodies

Desired gene segments or expression plasmids were synthesized at Geneart AG (Regensburg, Germany) from synthetic oligonucleotides and PCR products by automated gene synthesis. The DNA sequences encoding the variable heavy and light chain regions of the anti-IL-33 and anti-IL-6 binders were cloned into mammalian expression vectors using conventional cloning techniques. Recombinant antibody production was performed by transient transfection of EXPI293™ Cells in a defined, serum-free medium. For transfection, EXPIFECTAMINE™ 293 Transfection Kit was used (Gibco) according to the manufacturer's instructions. Cell culture supernatants were harvested 5-7 days after transfection by centrifugation and subsequent filtration (0.2 m filter), and proteins were purified from the harvested supernatant by standard methods as indicated below.

### Purification of Bispecific Antibodies

Proteins were purified from cell culture supernatants referring to standard protocols. For example, Fc containing proteins were purified from cell culture supernatants by Protein A-affinity chromatography. Elution was achieved at pH 3.0 followed by immediate pH neutralization of the sample. Pooled eluates from affinity chromatography were further purified by size exclusion chromatography using a HiLoad Superdex 200 26/60GL or XK50/70 (GE Healthcare, Sweden) column equilibrated with 20 mM histidine, 140 mM NaCl, pH 6.0. Alternatively, eluates from affinity chromatography were purified by ion exchange chromatography using a POROS XS column (Thermo Scientific, USA) prior to size exclusion chromatography. IgG containing fractions were pooled, concentrated to the required concentration using Vivaspin ultrafiltration devices (Sartorius Stedim Biotech S.A., France) and stored at −80° C.

Analytics of Bispecific Antibodies The concentrations of purified proteins were determined by measuring the absorption at 280 nm using the mass extinction coefficient calculated on the basis of the amino acid sequence according to Pace et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer). Determination of the aggregate content was performed by HPLC chromatography at 25° C. using analytical size-exclusion column (TSKgel G3000 SW XL or UP-SW3000, Tosoh Bioscience) equilibrated in running buffer (200 mM KH₂PO₄, 250 mM KCl pH 6.2, 0.0200 NaN₃).

Purification results are summarized in Table 3.

TABLE 3
Exemplary purification results for transiently expressed
bispecific anti-IL-33/anti-IL-6bispecific antibodies.

| | | analytical | | | non- | |
|---|---|---|---|---|---|---|
| | Expression | SEC after | | | reduced | |
| | yield after | capture step | | analytical | CE-SDS | |
| | capture step | [% monomer | purification | SEC | [% main | |
| | [mg/L] | aSEC] | yield [mg/L] | [% monomer] | peak] | LC-MS |
| P1AK9539 | 152.8 | 87 | 73.5 | 98.3 | 99 | identity and |
| | | | | | | integrity |
| | | | | | | confirmed |
| P1AM2967 | 80 | 66.5 | 26.9 | 98.9 | 100 | identity and |
| | | | | | | integrity |
| | | | | | | confirmed |
| P1AM2965 | 227.5 | 92.2 | 57 | 100 | 100 | identity and |
| | | | | | | integrity |
| | | | | | | confirmed |
| P1AM2966 | 187.5 | 95.7 | 39.13 | 100 | 100 | identity and |
| | | | | | | integrity |
| | | | | | | confirmed |
| P1AM3782 | 114.1 | 62.3 | 27.4 | 96.5 | 98.2 | identity and |
| | | | | | | integrity |
| | | | | | | confirmed |
| P1AM3779 | 152.6 | 60.2 | 47.7 | 96.5 | 99 | identity and |
| | | | | | | integrity |
| | | | | | | confirmed |
| P1AM3781 | 97.5 | 23 | —* | —* | —* | not |
| | | | | | | confirmed |
| | | | | | | (extensive |
| | | | | | | HC:LC |
| | | | | | | mispairing) |
| P1AM3780 | 115.5 | 92.3 | 108.8 | 99 | 98 | identity and |
| | | | | | | integrity |
| | | | | | | confirmed |

*purification discontinued due to low quality after capture

Antibody material that was captured by Protein A chromatography from cell culture supernatants was quantified via UV absorbance. 1+1 IgG-like variants P1AK9539, P1AM2965, P1AM2966 with a (VH/VL) crossover anti-IL-6 Fab on the “hole” subunit of the Fc domain showed capture yields of 152.8, 227.5 and 187.5 mg/L, respectively. Homogeneity of the captured material was comparable with 87, 92.9 and 95.7% monomer content, respectively, as judged by analytical size exclusion chromatography (aSEC). Surprisingly, switching the crossover to the anti-IL-33 Fab on the “knob”-side of the molecule significantly reduced both the capture yield (114.1 mg/ml for P1AM3782 compared to 152.8 mg/ml for P1AK9539 and 152.6 mg/ml for P1AM3779 compared to 227.5 mg/ml for P1AM2965) and the homogeneity (62.3% monomer for P1AM3782 compared to 87% for P1AK9539, and 60.2% monomer for P1AM3779 compared to 92.2% for P1AM2965) of the molecules.

P1AM3780, a 1+1 “inverted” IgG-like configuration with an uncrossed anti-IL-33 Fab fused to the C-terminus of the “knob” subunit of the Fc domain and a crossover anti-IL-6 Fab fused to the C-terminus of the “hole” subunit of the Fc domain (via 15 amino acid Gly-Ser linkers) showed a capture yield of 115.5 mg/ml and 92.3% monomer in aSEC.

Increasing the valency of IL-33 binding by reconfiguration of the antibody format led to a reduction in capture yields and in homogeneity. P1AM2967, a 2+1 IgG-like configuration with two uncrossed anti-IL-33 Fabs fused N-terminally to the Fc domain and one crossover anti-IL-6 Fab fused to the C-terminus of the “knob” subunit of the Fc domain (via a 20 amino acid Gly-Ser linker), showed a capture yield of 80 mg/L and 66.5% monomer in aSEC. P1AM3781, a 2+1 IgG-like configuration with an uncrossed anti-IL-33 Fab fused N-terminally to a crossover anti-IL-6 Fab on the “knob” subunit of the Fc domain (via a 9 amino acid Gly-Ser linker) in addition to an uncrossed anti-IL-33 Fab N-terminally of the “hole” subunit of the Fc domain, showed 97.5 mg/L capture yield and only 23% monomer in aSEC.

By subsequent chromatographic steps, the purity of the proteins was further improved by removal of side products that can be still contained in the captured material (i.e. heavy chain homodimers and heavy/light chain misparings). All bispecific antibodies were purified to high homogeneity with >95% monomer in aSEC and >95% purity in non-reduced SDS capillary electrophoresis (CE-SDS). One exception was P1AM3781, where purification was discontinued due to the high side-product content of the capture material.

Final purification yield was highest for P1AM3780 with 108.8 mg/L. For the 1+1 IgG-like variants P1AK9539 (PGLALA) and P1AM2965 (PGLALA+YTE), comparable final yields in the 70-60 mg range were obtained while slightly lower final yields in the range of 50-30 mg range were obtained for the 1+1 IgG-like variants P1AM3779, P1AM3782, P1AM2966 and the 2+1 variant P1AM2967.

In conclusion, initial production profiles were most promising for P1AM3780 (C-terminally fused Fab-Fragments), followed by the 1+1 IgG-like variants P1AK9539, P1AM2965 and P1AM2966. Surprisingly, switching the crossing to the anti-IL-33 Fab as in P1AM3782 and P1AM3779 led to less favorable results (lower capture yields and homogeneity and lower final purification yields). Similarly, 2+1 formats showed less favorable results, especially P1AM3781, which could not be produced in suitable quality.

### Physicochemical Characterization

Physicochemical properties (developability) of the bispecific antibodies were further assessed as described in the following and/or in Jarasch et al., J Pharm Sci (2015) 104, p. 1885-1898 (incorporated herein by reference in its entirety).

### A. Thermal Stability and Aggregation Propensity

For the assessment of thermal stability and aggregation propensity, molecules were exposed to increasing temperature in a controlled gradient and structural unfolding (Tₘ by intrinsic fluorescence) and/or aggregation propensity (T_{agg} by SLS) were determined.

Dynamic Light Scattering (DLS) measures the size distribution of particles in solution by analyzing the scattering of light. As proteins aggregate, their hydrodynamic radius increases, which can be detected by DLS. DLS can be used to determine the aggregation onset temperature (T_{agg}), which is the temperature at which aggregation begins. This is done by gradually heating the sample and monitoring changes in particle size. High thermal stability (Tₘ >60° C.) is generally desired for therapeutic antibodies, as it indicates robust structural integrity and folding efficiency. Thermal stability can be correlated with the folding efficiency of antibodies produced in various expression systems. Aggregation onset temperature (T_{agg}) is a valuable parameter for assessing colloidal stability. A T_{agg} greater than about 55° C. indicates good thermal and colloidal stability.

### B. Apparent Hydrophobicity

Apparent hydrophobicity was evaluated by determining the retention time on a hydrophobic interaction HPLC column in comparison with known high and low hydrophobicity standard molecules.

Hydrophobic Interaction Chromatography (HIC compares the apparent hydrophobicities of proteins in their native state. Unlike reversed-phase chromatography, which operates under denaturing conditions, HIC maintains the native conformation of proteins. Proteins are eluted based on their hydrophobic interactions with the chromatography matrix, allowing for the identification of unusually high hydrophobicity, which may drive aggregation or nonspecific interactions.

### C. PK Prediction

For PK prediction, the interaction with immobilized FcRn and Heparin on specific HPLC columns was evaluated. The corresponding retention times were compared to established thresholds based on known molecules with good and bad PK.

The interaction between the Fc region of antibodies and FcRn is pH-dependent, with strong binding at acidic pH (around pH 6 to 5, typical of endosomal environments) and weak binding at neutral pH (around pH 7.4, typical of the bloodstream). This pH-dependent binding ensures that antibodies are protected from lysosomal degradation and are recycled back into the circulation. Despite having identical Fc regions, different antibodies can exhibit varying retention times on an FcRn column, indicating that the variable regions (Fv domains) also influence FcRn binding. This suggests that FcRn column chromatography can reflect the dissociation behavior of antibodies from FcRn, which is relevant for their pharmacokinetics. Besides FcRn binding, it is essential to ensure that antibodies do not bind to off-targets ubiquitously present in vivo, as this can lead to rapid clearance. This can be tested by assessing the retention on a heparin column. Heparin is a highly sulfated glycosaminoglycan that interacts with various proteins, influencing their pharmacokinetics and biodistribution.

### D. Viscosity Assessment

Viscosity was assessed at different concentrations of the molecules of interest in a generic buffer system. The maximum feasible concentration was defined by a viscosity limit relevant for the target product profile and determined by fitting the available data to an appropriate model and applying the relevant threshold.

Viscosity is a critical parameter in the developability assessment of therapeutic antibodies, especially for formulations intended for subcutaneous (SC) administration, where high concentrations are often required. High viscosity can pose challenges in manufacturing, handling, and administration, affecting the overall patient experience and treatment efficacy.

Viscosity may be assessed by the Honeybun rheometer. The Honeybun Rheometer, also known as “Uncle,” is a rapid microvolume viscometer designed to measure the viscosity of protein solutions, including highly concentrated monoclonal antibodies. It is particularly useful in the developability assessment of therapeutic proteins, providing quick and reliable viscosity data with minimal sample volume. The Honeybun Rheometer operates based on the principles of rotational rheometry, where a sample is subjected to controlled shear stress or shear rate, and the resulting flow behavior is measured. The key components and steps involved in the operation of the Honeybun Rheometer are as follows: A protein solution, is loaded into the instrument's sample holder called the “Bun.” Each Bun requires only 35 μL of sample. The instrument is equipped with precise temperature control to ensure that measurements are conducted at the desired temperature. This is crucial for protein solutions, as viscosity can be highly temperature-dependent. The instrument measures the viscosity of the protein solutions by analyzing the flow behavior under controlled shear stress or shear rate. The viscosity is determined as a function of shear rate, providing insights into the sample's flow behavior under different conditions.

Results of the physicochemical characterization are summarized in Table 4 below. The results show, for example, that thermal stability of P1AM2965 (comprising the YTE modification in its Fc domain) is similar as for P1AK9539 and P1AM2966, which is unexpected given that the YTE modification is known to have a rather destabilizing effect (see e.g. Majumdar et al., mAbs 7, 84-95 (2015)). Lower T_{agg} is observed for P1AM3782 and P1AM3779, which have the (VH/VL) domain crossover in the anti-IL-33 Fab instead of the anti-IL-6 Fab.

### Example 4—HEK-Blue Activation Assay

### Materials and Methods

IL-33 Reporter HEK 293 Cells (Invivogen, Cat #hkb-hil33) and IL-6 Reporter HEK 293 Cells (Invivogen, Cat #hkb-hil6) were maintained in DMEM (Gibco, Cat #11965092) supplemented with 10% heat-inactivated FBS and HEK-BLUE™ Selection (Invivogen, Cat #hb-sel) in T150 cell culture flasks until 80% confluence. Cells were washed off using DPBS and seeded on 96-well plates (Corning, Cat #3595) at a density of 50.000 cells/well. Non-targeting control and blocking antibodies were incubated at a dose range of 0.004-100 nM with 1 ng/ml of hIL-6 (R&D Systems, Cat #206-IL/CF) or 1 ng/ml of hIL-33 wt (R&D Systems, Cat #3625-IL-010/CF) or 1 ng/mL hIL-33_mut (AdipoGen Life Sciences, Cat #AG-40B-0160-C100) for 45 minutes prior to the addition to HEK-BLUE™ IL-6 cells or HEK-BLUE™ IL-33 cells, respectively. Cells were incubated for 24 h in a humidified incubator (37° C., 5% CO₂). SEAP activity was assessed by adding 20 μL of cell culture supernatant to 180 μL QUANTI-BLUE™ Solution (Invivogen, Cat #rep-qbs) followed by a 2 h incubation at 37° C. Optical density at 620 nm was measured on a TECAN Spark 10M multimode plate reader.

### Results

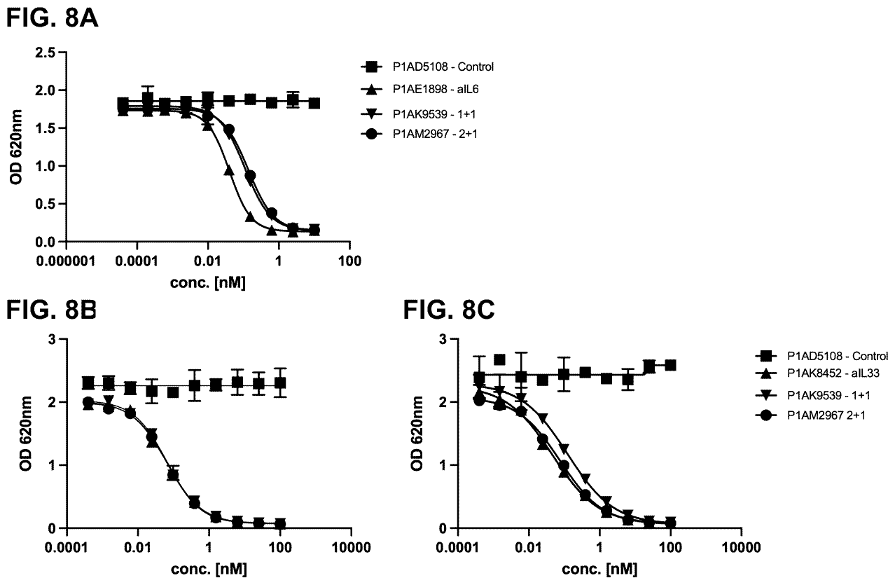

Commercially available cell-based reporter assays (HEK-BLUE™) were used to determine whether the combination of the two binders against IL-33 and against IL-6 into a bispecific antibody interfered with their ability to inhibit each cytokine. We tested our series of molecules, including bispecific antibodies with one antigen binding domain for each cytokine (referred to as 1+1; e.g. P1AK9539), and bispecific antibodies with two binding domains for IL-33 and one for IL-6 (referred to as 2+1; e.g. P1AM2967). A non-targeted human IgG₁ molecule (comprising the PG LALA Fc mutation) was used as control (P1AD5108). We tested a dose titration of the bispecific antibodies and the parent monoclonal IgG₁ antibodies in the respective assays, using the wild-type IL-6 (FIG. 8A) protein and a 10-point 1:4 dilution series of the blocking antibodies at concentrations ranging from 10 nM to 0.00004 nM, and two forms of the IL-33 cytokine and a 10-point 1:4 dilution series of the blocking antibodies at concentrations ranging from 100 nM to 0.0004 nM. The two forms of the IL-33 cytokine were the wild-type sequence (FIG. 8B) as well as a mutated sequence (FIG. 8C) in which cysteines have been mutated into serines (C208S, C227S, C232S, and C259S) to avoid oxidation that results in the inactivation of the ligand (Cohen et al., Nature Communications 6:8327 (2015)). Results are shown in FIGS. 8A-8C and Table 5 (for P1AK9539 and P1AM2967 and the monospecific antibodies), and in Table 6 (for the full series of molecules). The bispecific antibodies were equipotent to the monospecific anti-IL-33 antibody. The bispecific antibodies have an up to approximately 2-fold lower potency than the monospecific anti-IL-6 antibody, which is within an acceptable range of potency variation (Table 5).

TABLE 5
IC50 values from HEK-Blue assay.

| | | IL33_wt HEK- | IL33_mut HEK- | IL6 HEK- |
|---|---|---|---|---|
| | | blue assay | blue assay | blue assay |
| Molecule | | IC50 (nM) | IC50 (nM) | IC50 (nM) |
| P1AE1898 | aIL-6 | N/A | N/A | 0.04 |
| P1AK8452 | aIL-33 | 0.06 | 0.10 | N/A |
| P1AK9539 | 1 + 1 | 0.07 | 0.10 | 0.11 |
| | aIL-33 + | | | |
| | aIL-6 | | | |
| P1AM2967 | 2 + 1 | 0.07 | 0.06 | 0.14 |
| | aIL-33 + | | | |
| | aIL-6 | | | |

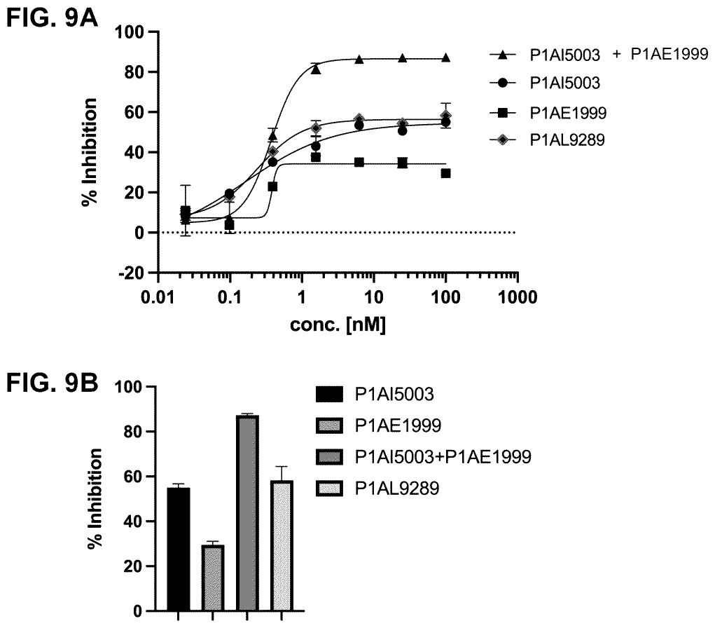

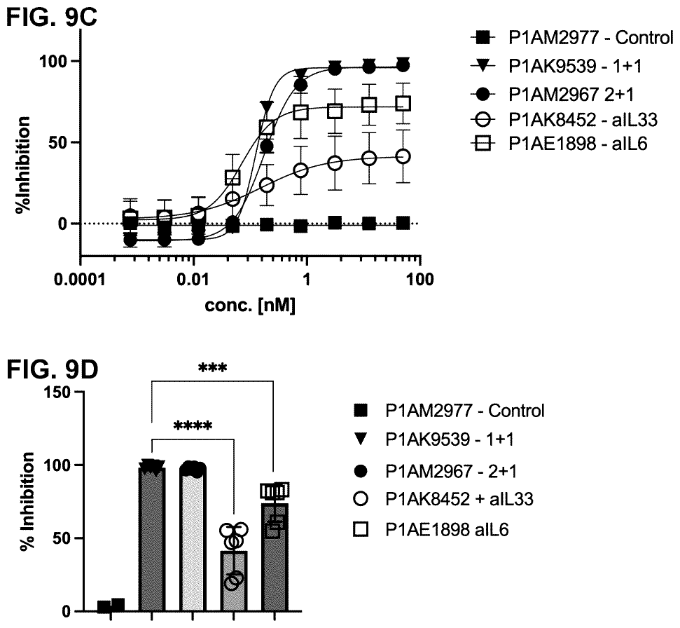

To investigate the potential redundancy of the mechanism by which IL-33 and IL-6 cause activation of endothelial cells resulting in ICAM-1 upregulation, we stimulated HUVECs with a combination of 4 ng/ml IL-33 (approximate EC30) and 11 ng/ml IL-6+22 ng/ml sIL-6R (approximate EC40) for 48 h and simultaneously treated with monospecific anti-IL-33 or anti-IL-6 blocking antibodies as well as the 1+1 and 2+1 anti-IL-33/anti-IL-6 bispecific antibodies, in a 10-point 1:4 dilution series at concentrations ranging from 50 nM to 0.000763 nM. A non-targeted human IgG₁ molecule (comprising the PG LALA and LS Fc mutations) was used as control (P1AM2977). Results are shown in FIGS. 9A-9D (for P1AK9539 and P1AM2967 and the monospecific antibodies), and in Table 6 (for the full series of molecules). The assay was performed as described in Example 1 above. The results showed that both cytokines needed to be inhibited in order to completely prevent the activation of the HUVECs and the upregulation of ICAM-1, both when a monospecific IL-33 blocking antibody (an IgG1 antibody with the VH and VL sequences of SEQ ID NOs 43 and 44, respectively (and the PG LALA Fc mutations); P1AI5003) was combined with a monospecific IL-6 blocking antibody (a Fab molecule with the VH and VL sequences of SEQ ID NOs 4 and 8, respectively; P1AE1999) (FIG. 9A, FIG. 9B) and when the bispecific antibody was applied (FIG. 9C, FIG. 9D). We can thus conclude that IL-33 and IL-6/sIL-6R act upon HUVECs through non-redundant pathways and that the inhibition of both cytokines is required to fully prevent endothelial cell activation.

The IC50 concentrations and the percent inhibition at the highest concentration assayed of all molecules tested in the HEK-Blue assays using either IL-6, WT IL-33, or mutant IL-33, as well as the ICAM-1 upregulation assay are listed in Table 6. All molecules were tested as a 10-point 1:4 dilution series ranging from 10 nM to 0.0004 nM in the IL-6 HEK-Blue assays; and a 10-point 1:4 dilution series ranging from 100 nM to 0.004 nM in the IL-33 HEK-Blue assays using wild-type IL-33 or mutant IL-33. P1AM3781 could not be satisfactorily expressed and purified. P1AM3780 had significantly lower potency in blocking IL-6 signaling, and to a lesser extent also in blocking IL-33 signaling, in the HEK-Blue assay while P1AM3779 was unable to suppress more that 80% of ICAM-1 upregulation at 100 nM. The rest of the antibodies had similar potencies.

### Example 5—Pharmacokinetics and Tissue Partitioning

### Materials and Methods

### A. Pharmacokinetic Profiling

The pharmacokinetic profile of P1AK9539, P1AM2967, P1AM2965 and P1AM2966, was assessed in female scid FcRn−/− hFcRn (32) Tg mice following a single intravenous dose of 5 mg/kg. The study was conducted at Selvita Ltd. facilities in Zagreb, Croatia. All procedures were conducted in accordance with 2010/63/EU and national legislation regulating the use of laboratory animals in scientific research. The Institutional Committee on Animal Research Ethics (CARE-Zg) ensured that animal welfare was not compromised.

Female scid FcRn−/− hFcRn (32) Tg mice, aged 23-28 weeks, were obtained from The Jackson Laboratory through Charles River (JAX stock #018441). The animals were acclimatized for a minimum of 5 days before the start of the study. The room conditions were maintained at 22° C.±2° C. with 55%+10% relative humidity and 15-20 air changes per hour. A 12-hour light/dark cycle was followed.

The test articles were formulated for intravenous administration. Each mouse received a single intravenous dose of 5 mg/kg via the lateral tail vein. Animals were observed before and after administration and once daily for any clinical signs of adverse effects. Blood samples were collected at 0, 17, 7, 24, 48, 72, 168, 336, 408, and 504 hours from the lateral tail vein, and processed to serum. On day 4, animals were euthanized using a CO₂ gas chamber.

### B. ECLIA Analysis

Serum samples of scid FcRn−/− hFcRn (32) Tg mice treated with P1AK9539, P1AM2967, P1AM2965 or P1AM2966 were analysed with a generic electrochemiluminescence immunoassay (ECLIA) method specific for hCH2 domains using a cobas e411 instrument under non-GLP conditions.

Briefly, test samples of P1AK9539, P1AM2967, P1AM2965 or P1AM2966, first detection antibody mAb<H-Fcg-pan>M-R10Z8E9-F(ab′)2-Bi, second detection antibody mAb<H-Fcg-pan>M-R10Z8E9-F(ab′)2-hyBP-Ru and streptavidin (SA)-beads were added to a detection vessel and incubated for 9 minutes in each step. Finally, the SA-beads-bound complex was detected by a measuring cell which numbers the counts of SA-beads in repeat. The counts were proportional to the analyte concentration in the test sample.

The calibration range of the standard curves for P1AK9539, P1AM2967, P1AM2965 and P1AM2966 was 0.69 ng/mL to 1′500 ng/mL assay concentration in 1% C57BL/6 mouse serum.

For analysis of serum samples, standard curves, quality controls, and sample dilutions were done in assay buffer, including C57BL/6 mouse serum resulting in 1% matrix concentration. Serum samples were analysed in one dilution (1:100 to 1:200). The analytical sensitivity was 68.59 ng/mL in 100% serum. Experimental serum samples with a concentration below 68.59 ng/mL were annotated as <68.59 ng/mL and are below limit of quantification.

The pharmacokinetic parameters were calculated from the time course of measured drug concentrations using standard noncompartmental analysis (NCA).

### C. Assessment of Biodistribution

The lung partitioning of P1AK9539, P1AM2967, P1AM2965 and P1AM2966 was assessed in male mice of the C57BL/6J strain, supplied by Charles River Laboratories following a single intravenous dose of 5 mg/kg. The study was conducted at F. Hoffmann-La Roche Ltd. The study design adhered to general animal health and welfare guidelines. The test facility was accredited by the Association for Assessment and Accreditation of Laboratory Animal Care (AAALAC).

Male C57BL/6J mice aged approximately 8 weeks were obtained from Charles River Laboratories. The animals were acclimatized for a minimum of 5 days before the start of the study. The room conditions were maintained with a 12-12 hour light/dark cycle, a temperature range of 20-24° C.±1° C., and a humidity level of 55%+15% to ensure the well-being of the animals throughout the study.

The test articles were formulated for intravenous administration. Animals were grouped, and each mouse received a single intravenous dose of 5 mg/kg via the lateral tail vein. Animals were observed before and after administration and twice daily for any clinical signs of adverse effects.

In-life blood samples were collected at 4, 24, and 48 hours from the lateral tail vein and processed to serum. On the scheduled day of the necropsy, 3, 6, and 120 hours after test article administration, animals were anesthetized with pentobarbital and euthanized without recovery from anesthesia by decapitation or bilateral pneumothorax. Blood and the left lobes of the lung, the kidney, and the left quadriceps tissues were collected. The blood was processed as described for tail-vain collected samples, and tissue samples were flash-frozen in 2 mL sterile DNAse and RNAse-free Eppendorf tubes.

D. Tissue Processing Prior to analysis, tissue samples were mechanically lysed in 500 μL of tissue extraction buffer (Tissue Extraction Reagent I; Invitrogen; Id. no. FNN0071) containing protease inhibitors (cOmplete; Sigma-Aldrich; Id. no. 116974980001) using the MagNA Lyser Homogenisator for 20 sec at 6′500 rpm. The following tissue weights were subjected to lysis: kidney 73-235 mg; left lobes of lung 42-66 mg and left quadriceps muscle 80-120 mg. After the lysis step in the MagNA Lyser Homogenisator, the lysed samples were centrifugated at 11,000×g, and supernatants were transferred to sterile Eppendorf tubes.

### E. ECLIA Analysis

Tissue lysates and collected serum samples of C57BL/6J mice treated with P1AK9539, P1AM2967, P1AM2965 or P1AM2966 were analysed using a generic electrochemiluminescence immunoassay (ECLIA) method as described above.

All results obtained from tissue lysates were normalized to the tissue weight and reported as a final concentration in ng/g tissue weight used for lysis.

Plasma drug concentrations and terminal tissue concentrations were sampled at 5-7 time points and 2-3 time points, respectively, for each compound. Concentration for tissue interstitial space was back-calculated using the physiologically known fraction of interstitial volume fraction. Plasma and tissue interstitium concentration data was fitted to a standard three-compartment PK model with physiological plasma, peripheral interstitial and tissue interstitial volumes to estimate the tissue partitioning coefficient.

### Results

Mutations in the Fc region of monoclonal antibodies that increase binding affinity to the neonatal Fc receptor (FcRn) have been shown to extend the antibody's half-life in the bloodstream, reducing the frequency of dosing required for therapeutic efficacy. This can be beneficial by contributing to better patient compliance and reducing the overall cost of the treatment both in terms of the drug itself and the associated healthcare costs (e.g., fewer clinic visits for administration). By maintaining higher and more stable plasma concentrations of the antibody, the therapeutic efficacy can be improved. This is particularly important for conditions where consistent antibody levels are crucial for effective treatment. The extended half-life can provide a broader therapeutic window, allowing for more flexibility in dosing schedules and potentially reducing the risk of adverse effects associated with peak drug concentrations. The extended half-life may also facilitate the development of subcutaneous formulations, which can be more convenient for patients compared to intravenous administration.

We thus assessed whether or not the addition of two sets of Fc mutations (YTE: M252Y, S254T, T256E, and LS: M428L and N434S) would have a beneficial impact on the pharmacokinetic properties of the 1+1 bispecific antibody P1AK9539. Using transgenic mice engineered to endogenously express the human FcRn sequence, we determined that both the YTE and LS mutations were able to significantly increase serum half-life (with YTE providing the longest half-life). Unexpectedly, the 2+1 bispecfiic antibody P1AM2967 had a significantly faster clearance and thus a lower half-life than the 1+1 bispecific antibodies. The lung partitioning tested in a separate study using WT mice was comparable amongst molecules. Results are summarized in Table 7.

TABLE 7
Pharmacokinetic and tissue partitioning values.

| | Cmax | AUCinf | Clearance | Half life | |
|---|---|---|---|---|---|
| Molecule | (μg/mL) | (day*μg/mL) | (mL/day/kg) | (days) | Kp_{lung}* |
| P1AK9539 | 118 | 793 | 6.35 | 10.9 | 0.25 |
| 1 + 1 | [3.43%] | [9.4%] | [9.42%] | | |
| aIL33 + aIL6 | | | | | |
| P1AM2967 | 126 | 103 | 48.5 | 2.07 | 0.20 |
| 2 + 1 | [4.52%] | [4.82%] | [4.88%] | | |
| aIL33 + aIL6 | | | | | |
| P1AM2965 | 132 | 1370 | 3.65 | 17.2 | 0.25 |
| 1 + 1 | [11.2%] | [5.9%] | [6.05%] | | |
| aIL33 + aIL6 | | | | | |
| YTE | | | | | |
| P1AM2966 | 138 | 1340 | 3.75 | 15.6 | 0.30 |
| 1 + 1 | [12.2%] | [10.9%] | [10.5%] | | |
| aIL33 + aIL6 | | | | | |
| LS | | | | | |

*Kp values generated from separate biodistribution study in WT mice

TABLE 4
Physicochemical analysis of anti-IL-33/anti-IL-6 bispecific antibodies produced in Example 3.

| | expected | | | | | | | | |
|---|---|---|---|---|---|---|---|---|---|
| Parameter | value | P1AK9539 | P1AM2965 | P1AM2966 | P1AM2967 | P1AM3782 | P1AM3779 | P1AM3781 | P1AM3780 |
| Thermal | T_{agg} > 64° C. | 64.6 | 63.5 | 64.5 | 65.7 | 58 | 56.7 | n.d | 64.6 |
| stability | | | | | | | | | |
| (DLS T_{agg}/ | | | | | | | | | |
| DLS Tₘ) | | | | | | | | | |
| Apparent | rel. ret. < | 0.94 | 0.95 | 0.94 | 0.76 | 0.94 | 0.95 | n.d | 0.43 |
| hydrophobicity | 0.35 | | | | | | | | |
| (HIC) * | | | | | | | | | |
| FcRn | Fc wt vs Fc | 0.89 | 5 | 6.38 | 0.95 | 0.86 | 4.95 | n.d | 0.34 |
| affinity | engineered | | (YTE) | (LS) | | | (YTE) | | |
| column | | | | | | | | | |
| Heparin | 0 < rel. ret. | 0.39 | 0.24 | 0.31 | 0.41 | 0.33 | 0.25 | n.d | 0.26 |
| affinity | time > 0.8 | | | | | | | | |
| chroma- | | | | | | | | | |
| tography | | | | | | | | | |
| Viscosity | Highest | 157 | 165 | 161 | n.d. | n.d. | n.d. | n.d | n.d. |
| | conc. | | | | | | | | |
| | below 20 cp | | | | | | | | |
| | (mg/ml) | | | | | | | | |

* values are attributed to the anti-IL-6 binder

TABLE 6
IC50 values from HEK-Blue assay and EC50 values from ICAM-1 upregulation assay.

| | | | | | | | ICAM-1 |
|---|---|---|---|---|---|---|---|
| | | | | | | | upegulation |
| | | | IL33% | | IL33% | ICAM-1 | % |
| | | IL33 | inhibition | IL33 | inhibition | upregulation | inhibition |
| | | IC50 | at highest | IC50 | at highest | IC50 | at highest |
| | IL6 IC50 | (nM) | dose | (nM) | dose | (nM) | dose |
| | (nM) | (signaling) | (signaling) | (signaling) | (signaling) | (trans | (trans |
| Molecule | (signaling) | wt IL33 | wt IL33 | mut IL33 | mut IL33 | inhibition) | inhibition) |
| P1AK9539 | 0.11 | 0.07 | 100 | 0.10 | 100 | 0.13 | 89.0 |
| P1AM2967 | 0.14 | 0.07 | 100 | 0.06 | 100 | 0.18 | 95.5 |
| P1AM2965 | 0.06 | 0.10 | 100 | 0.00 | 100 | 0.23 | 85.6 |
| P1AM2966 | 0.06 | 0.08 | 100 | 0.05 | 100 | 0.14 | 96.3 |
| P1AM3782 | 0.09 | 0.07 | 100 | 0.07 | 100 | 0.15 | 88.7 |
| P1AM3779 | 0.10 | 0.08 | 100 | 0.08 | 100 | 0.17 | 74.6 |
| P1AM3781 | n/a | n/a | n/a | n/a | n/a | n/a | n/a |
| P1AM3780 | 1.77 | 0.45 | 100 | 0.45 | 100 | 0.25 | 82.3 |

In conclusion, taking together the above described results comparing the various bispecific antibody molecules, the 1+1 IgG-like molecules P1AK9539, P1AM2965 and P1AM2966 provide the most favorable properties, including produceability, stability, as well as biological function. These molecules also exhibit good pharmacokinetic and lung partitioning profiles, with P1AM2965 and P1AM2966 additionally providing prolonged serum half-life due to their respective Fc domain mutations.

### Example 6—Bulk mRNA Sequencing of HUVEC Cells Treated with Cytokines

### Materials and Methods

### A. Cytokine Stimulation, RNA Extraction, and Sequencing

Primary Human Umbilical Vein Endothelial Cells (HUVEC) from pooled donors were obtained from Promocell (Cat #C-12203, LOT #511Z011 and LOT #505Z025) and expanded in Endothelial Cell Growth Medium 2 (Promocell, Cat #C-22011) on T150 cell culture flasks coated with Attachment Factor Protein (Gibco, Cat #S006100) until 80% confluence. HUVECs were detached using STEMPRO™ ACCUTASE™ Cell Dissociation Reagent (Gibco, Cat #A1110501), resuspended in endothelial medium, counted using Trypan Blue exclusion, and resuspended to a cell seeding density of 2×10⁵ cells/mL. Cells were seeded onto standard TC-treated 24-well plates, 1 mL each well, and allowed to attach at 37° C. in a humidified incubator overnight. At this stage, 50 ng/mL IL-33 (AdipoGen Life Sciences, Cat #AG-40B-0160-C100) or 50 ng/mL IL-6 and 100 ng/mL IL-6R (R&D Systems, Cat #206-IL/CF and Cat #227-SR-025) or a combination of all three was added to the cells for 24 h at 37° C. Cells were lysed in the well in 350 μl MagnaPure external lysis buffer (Roche, Cat #06374913001) and RNA was subsequently extracted using the MagnaPure cellular RNA large Volume kit (Roche, Cat #05467535001) on the MagnaPure 96-well instrument. Elution was done in 50 μl. RNA was quantified using the Qubit RNA High Sensitivity assay (Invitrogen, Cat #Q32855) and the RNA integrity number was determined using the Tapestation4200 and the High Sensitivity RNA screentape (Agilent, Cat #5057-5579) and the HS RNA reagents (Agilent, Cat #5067-5580). Libraries were generated using the Illumina total RNA prep with Ribo Zero Plus (Illumina, Cat #20040529) and 110 ng RNA input for all samples. The libraries were generated following the manufacturer's instructions. Final libraries were quantified using the Qubit 1×dsDNA HS assay (Invitrogen, Cat #Q33231) and the fragment size distribution was analyzed using the Tapestation 4200 and the D1000 screen tape (Agilent, Cat #5067-5582) and the D1000 reagents (Agilent, Cat #5067-5583). Libraries were diluted to 5 nM each and pooled in equimolar amounts. The final pool of libraries was diluted to 0.6 nM and a final spike in of 1% PhiX was used. Sequencing was done on the NovaSeqX using 1 lane (lane 3) of a 10B flow cell and the 100 cycle kit. Read configuration: 51-10-10-51.

### B. RNA Sequencing Data Processing and Analysis

Raw sequencing data underwent base calling using bcl-convert (v4.3.13, Illumina) (R1). The quality of the resulting FASTQ files was assessed using FastQC (v0.12.1) (R2). Adapter trimming and quality filtering were performed with fastp (v0.23.4) (R3) using default parameters.

Paired-end RNA-seq reads were aligned to the GRCh38.p14 build of the human genome using STAR aligner (v2.7.11b) (R4) with default mapping parameters. Gene-level quantification was performed using featureCounts from the Subread package (v2.0.6) (R5) based on the Ensembl v112 reference annotation, with parameters set to exclude low-quality mapping reads (-Q 10) and perform strand-specific quantification (-s 2).

Quality control metrics were generated using samtools stats (v1.13) (R6), Picard CollectRnaSeqMetrics (v3.1.1) (R7), and RSeQC (v5.0.2) (R8) (bam_stat.py and infer_experiment.py). Results were aggregated and visualized using MultiQC (v1.21) (R9).

### C. PCA Analysis

For exploratory data analysis, gene expression values were transformed to log 2-counts per million (log-CPM) using the cpm function, incorporating the TMM normalization factors. Principal Component Analysis (PCA) was performed to visualize sample relationships using PCAtools (version 2.14.0). To reduce noise and focus on the primary sources of variation, the PCA was conducted on a subset of the 500 most highly variable genes. This gene set was identified by calculating the variance for each gene across all samples from the original raw count matrix. The resulting normalized, log-CPM expression matrix for these 500 genes was then z-scaled and then used as input for PCAtools (version 2.14.0) package [3], with sample metadata included for visualization.

### D. Differential Expression Analysis

DEG analysis was performed using DESeq2 (version 1.40.1) across treatment groups using the individual as confounding factors (because we had 3 replicates per individual) to find genes between the different treatments. For follow-up analysis, only genes were used that had a log 2 fold change bigger than 1 or smaller than −1 and an adjusted p-value smaller than 0.05.

### E. Visualisation of Data

PCA was visualized using built in functions from PCAtools (version 2.14.0). Venn diagram was obtained using ggVennDiagram (version 1.5.4).

### Results

In order to characterize the molecular pathways that drive endothelial dysfunction when exposed to IL-33 and IL-6 complexed with sIL-6R to induce trans-IL-6 signaling, or their combination, we employed bulk mRNA sequencing (bulk mRNAseq). Treatment with high concentrations of each cytokine ensured maximal activation of the individual pathways.

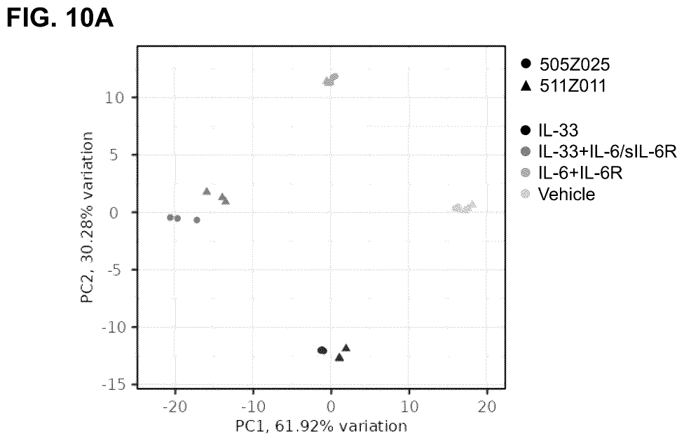

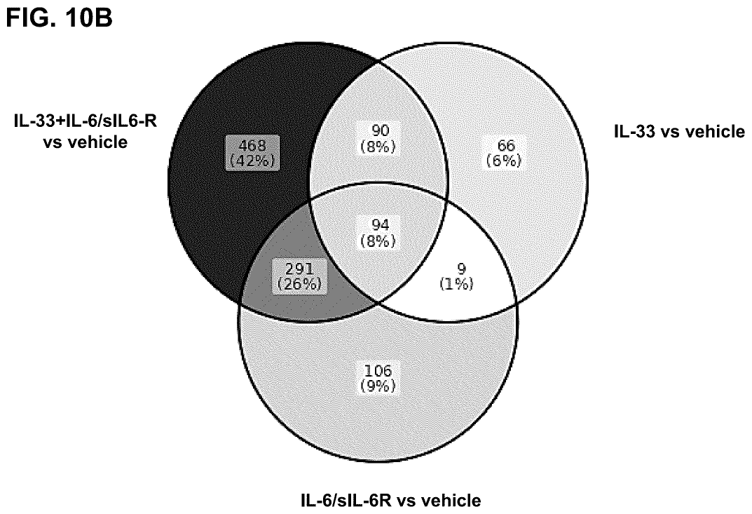

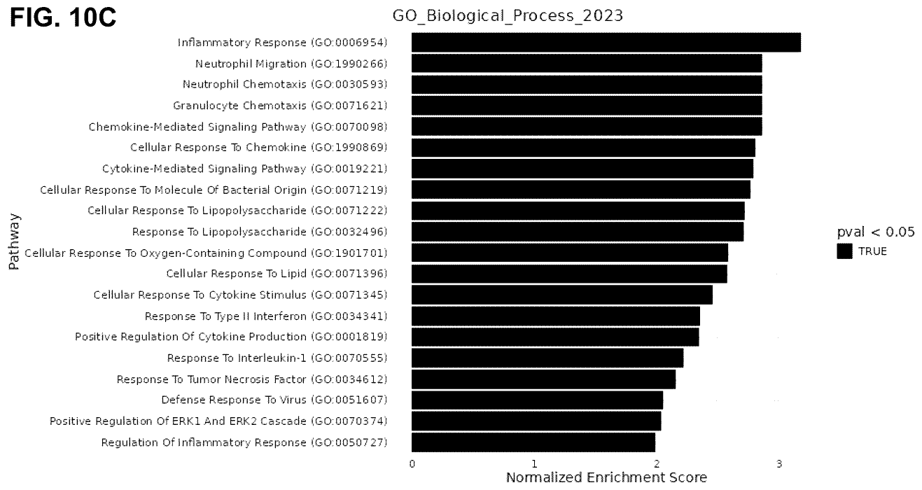

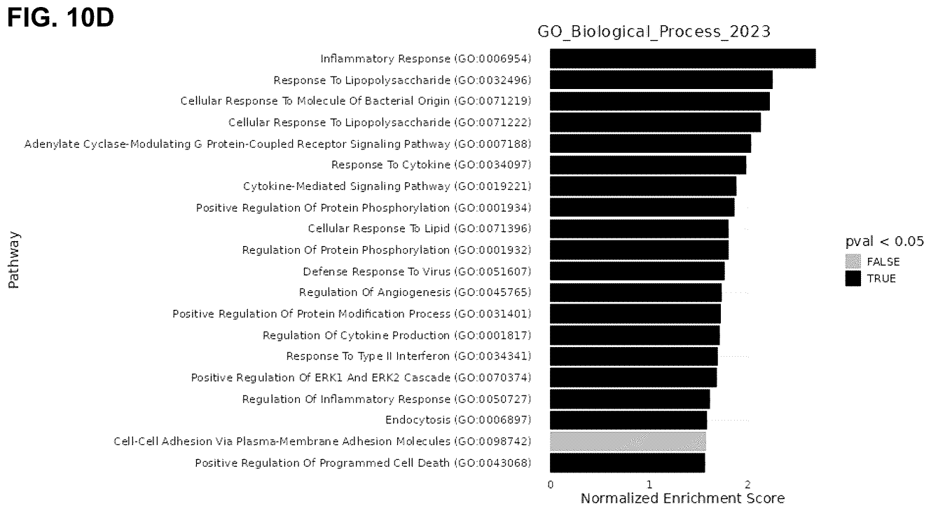

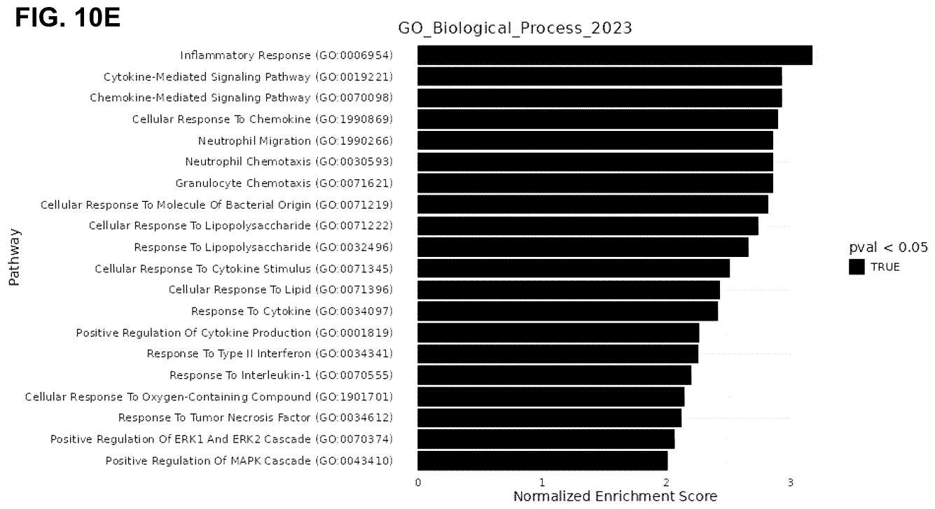

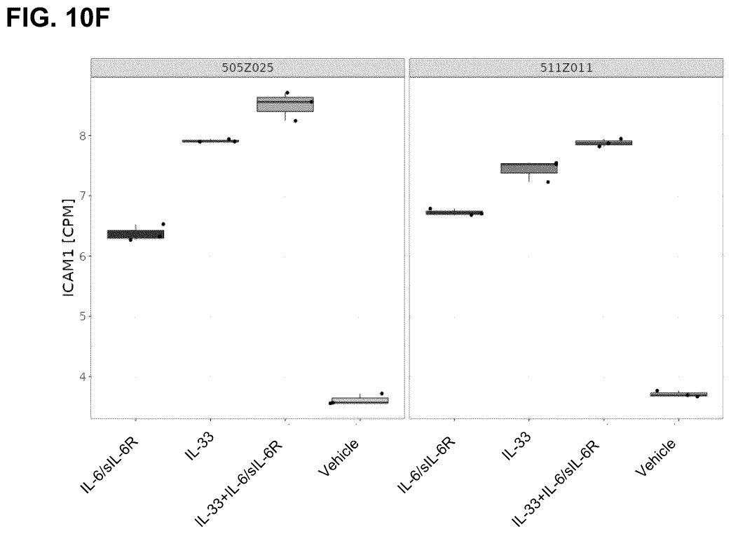

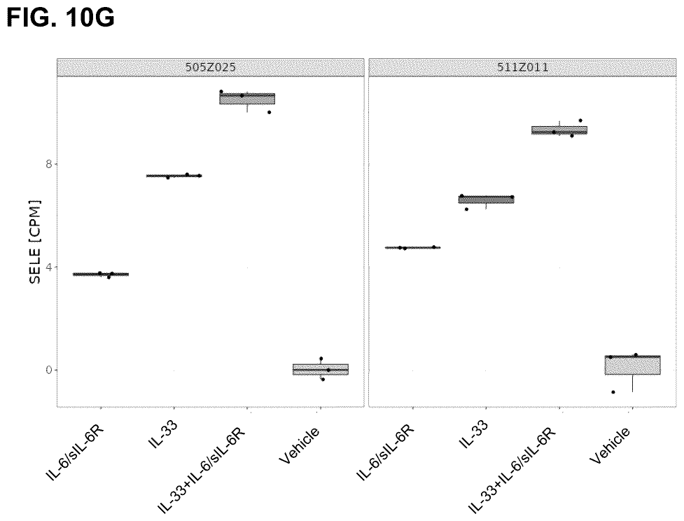

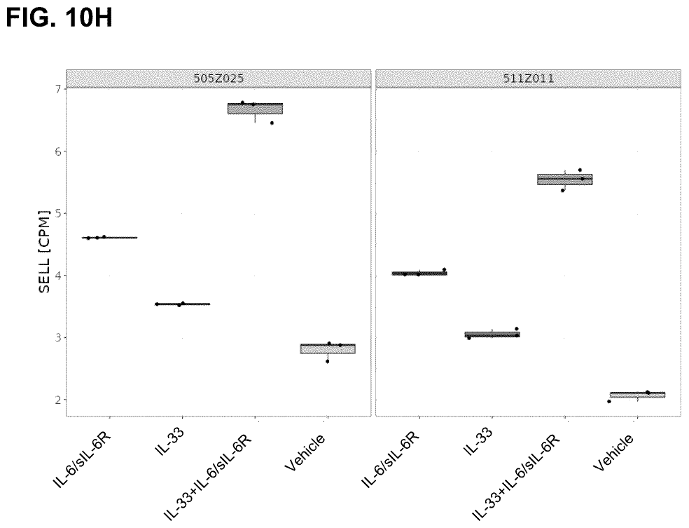

For exploratory data analysis and to determine the differential effect of each treatment, we performed Principal Component Analysis (PCA) (FIG. 10A). The close proximity of the two donor pools on the PCA plot exemplifies the similarity of the effects of the three cytokine treatment conditions on the two HUVEC lots. The distance between the treatment conditions and the vehicle control treatment conditions demonstrates the differential effects of each of these treatments. To further understand the magnitude of these differences, we created a Venn diagram of the differentially expressed genes (DEGs) between each of the individual cytokine treatment conditions and the vehicle control (FIG. 10B). Not surprisingly, the combined treatment of IL-33 and IL-6/sIL-6R had the largest number of DEGs, with 943, with IL-6/sIL-6R treatment having 500 DEGs, and IL-33 treatment having 256 DEGs. While a significant amount of those genes overlapped with the individual treatments, 42% of all DEGs were specific to the combined IL-33+IL-6/sIL-6R treatment.

Pathway analysis performed on the DEGs identified via bulk mRNA sequencing, which examined the biological processes induced by each treatment, demonstrated distinct and combinatorial activation patterns in HUVECs. For cells treated individually with IL-33, the top pathways identified included inflammatory response and neutrophil migration and chemotaxis (FIG. 10C).

Similarly, exposure to IL-6 induced key pathways such as inflammatory response, response to LPS, and cellular response to molecules from bacteria (FIG. 10D). Critically, the combined IL-33 and IL-6/sIL-6R treatment resulted in the identification of a complex set of molecular pathways (FIG. 10E) reflecting a combinatorial effect, encompassing inflammatory response, cytokine-mediated signaling pathway, chemokine-mediated signaling pathway, cellular responses to chemokines and cytokines, neutrophil migration and chemotaxis, and response to LPS. This overall pathway profile demonstrates that the combined treatment significantly influences HUVEC cells in a manner specifically related to immune cell, particularly neutrophil, migration.

More specifically, the adhesion molecules known to contribute to neutrophil transmigration through an endothelial layer (ICAM-1, SELE, SELL), were upregulated by the combined treatment with both cytokines to a level higher than by each individual cytokine treatment by itself (FIG. 10F, FIG. 10G, FIG. 10H).

### Example 7—Neutrophil Migration Through a HUVEC Vascular Tube and Inhibition by Therapeutic Antibodies

### Materials and Methods

### A. Endothelial Tubule Formation

Primary Human Umbilical Vein Endothelial Cells (HUVEC) from pooled donors were obtained from Promocell (Cat #C-12203, LOT #511Z011) and expanded in Endothelial Cell Growth Medium 2 (Promocell, Cat #C-22011) on T150 cell culture flasks coated with Attachment Factor Protein (Gibco, Cat #S006100) until 80% confluence.

MIMETAS ORGANOPLATE® 3-lane 64 (MIMETAS, Cat #6405-400-B) were used for this study. To form the extracellular matrix barrier, bovine collagen (Fibricol, Cellsystems, Cat #5133-20ML) was first diluted with 0.02 M acidic acid to have 5 mg/mL collagen which was mixed with HEPES and 37 g/L NaHCO₃ at an 8:1:1 ratio to form a 4 mg/mL collagen solution. The components were carefully mixed well and 20 μL of the gel solution was seeded into each chip. The gel was allowed to polymerize for 1 hour at 37° C. in a humidified incubator. 30 μL PBS was added to each gel inlet to hydrate the ECM layer prior to HUVEC seeding.

HUVECs were detached using STEMPRO™ ACCUTASE™ Cell Dissociation Reagent (Gibco, Cat #A1110501), HUVECs were detached from the flask, resuspended in endothelial medium, counted using Trypan Blue exclusion, and resuspended to a cell seeding density of 15×10⁶ cells/mL. To each right bottom port, 50 μL of medium was added prior to dispensing 2 μL of cell suspension into the right top inlet port using the automatic repeater pipette. The cell suspension was regularly mixed in order to ensure homogenous cell seeding density. The OrganoPlates were placed with the lid, forming a 75-degree angle against the plate stand, and left in this orientation for 2 h to allow cells to attach. After cell attachment, 50 μL of the endothelial medium was added into the top medium inlet. The plate was then placed on the ORGANOFLOW® in a humidified incubator set to an inclination of 14° and an interval of 8 minutes for 48-72 h to allow for tubule formation.

### B. HUVEC Stimulation/Barrier Challenge

After successful tubule formation, HUVEC growth medium was carefully replaced by assay medium consisting of AIM V Medium (Gibco, Cat #12055091), supplemented with 5 ng/mL Recombinant Human FGF-basic (Peprotech, Cat #100-18B) and 5 ng/mL Recombinant Human VEGF165 (Peprotech, Cat #100-20). At this stage, 50 ng/mL IL-33 (AdipoGen Life Sciences, Cat #AG-40B-0160-C100) or 50 ng/mL IL-6 and 100 ng/mL IL-6R (R&D Systems, Cat #206-IL/CF and Cat #227-SR-025) or a combination of all three in the presence or absence of inhibitory anti-IL-33 or anti-IL-6 monospecific antibodies (100 nM) or anti-IL-33/anti-IL-6 bispecific antibody (200 nM) was added to the HUVEC tubule and plates were placed on the ORGANOFLOW® in a humidified incubator set to an inclination of 140 and an interval of 8 minutes for 24 hours.

### C. Neutrophil Isolation and Seeding

Neutrophils were isolated from fresh whole blood collected in K2/EDTA vials from healthy donors using the EASYSEP™ Direct Human Neutrophil Isolation Kit (STEMCELL, Cat #19666) was carried out according to the Fully Automated ROBOSEP™ Protocol.

Following isolation, cells were centrifuged at 250×g for 3 minutes at room temperature and cells were resuspended in assay medium (AIM V medium supplemented with 5 ng/mL FGF and 5 ng VEGF) at a concentration of 3×10⁶ cells/mL. 10 μL of the cell suspension (30,000 cells/chip) was added to the designated chips. Finally, 50 nM CXCL8 was added to the left inlets and outlets of the ORGANOPLATE®, and plates were placed on the ORGANOFLOW® in a humidified incubator set to an inclination of 14° and an interval of 8 minutes for 24 hours.

### D. Immunofluorescent Staining

The staining was based on the MIMETAS protocol with minor adaptations. Perfusion during incubation steps was maintained by placing the ORGANOPLATE® on the MIMETAS ORGANOFLOW® Rocker at a 7° angle, switching sides every 2 minutes.

Following the migration period, cells were fixed with 4% paraformaldehyde (PFA) for 10 minutes. For the ORGANOPLATE® 3-lane 64 format, 100 μL of fixative was added to the tubule inlet and 50 μL to all other inlets and outlets to induce flow. After fixation, chips were washed 2× (5 min each) with PBS using the same volume scheme. Cells were permeabilized for 10 minutes using the Permeabilization Buffer (0.3% Triton X-100 in PBS). Chips were then washed 2× for 5 minutes with PBS. Blocking was performed for 30-45 minutes using Blocking Buffer (2% Normal Goat serum, 2% Bovine Serum Albumin, and 0.1% Tween 20 in PBS). The volume scheme for wash, permeabilization, and blocking steps used 100 μL for the right perfusion channel inlet and 50 L for all other inlets and outlets. Alexa Flour 488 Phalloidin Labeling Probe (Thermo, Cat #A12379) and Neutrophil Elastase/ELA2 Antibody (NP57) (R&D, Cat #NBP2-50529) were diluted in Blocking Buffer and added to the chips and incubated overnight at room temperature on the ORGANOFLOW® rocker at a 7° angle, switching sides every 2 minutes. The volume scheme for antibody incubation steps used 25 L for the right perfusion channel inlet and outlet and 15 L for left perfusion channel inlet and outlet. Following primary antibody incubation, chips were washed 3× for 30 minutes each following the volume scheme described above for Fixation, Permeabilization and Blocking. ALEXA FLUOR® 647 AFFINIPURE™ Fab2 Fragment Donkey Anti-Mouse IgG (Jackson Immuno Research, Cat #715-606-150) was diluted in blocking buffer, added to the chips and incubated at room temperature for 3 hours on the ORGANOFLOW® rocker at a 7° angle, switching sides every 2 minutes. Following secondary antibody incubation, chips were washed 3× for 30 minutes each. After the final wash all wells were filled up with 50 μL of PBS for image acquisition.

Images were acquired on an Opera Phenix System with a 10× objective and analyzed using PerkinElmer's Harmony software.

### E. Quantitative Analysis of CCL2 (MCP-1)

The concentration of C-C motif chemokine ligand 2 (CCL2 or MCP-1) in cell culture supernatants was quantified using the Simple Plex Technology (ProteinSimple) on an Ella instrument. Following the migration period, medium from the right top and bottom inlet port was collected and CCL2 was quantified using the Simple Plex Human CCL2 Cartridge (Cat #SPCKB-PS-001108) according to the manufacturer's instructions. Samples were diluted 1:20 in kit-specific sample buffer and raw data were collected and analyzed using the Simple Plex Runner 5.0.0.4 Software. The software generated a standard curve using a four-parameter logistic (4-PL) regression model and automatically calculated the absolute concentration of CCL2 (expressed in pg/mL) for each sample.

### F. Statistical Methods

Statistical analysis was performed using Graphpad Prism v10.

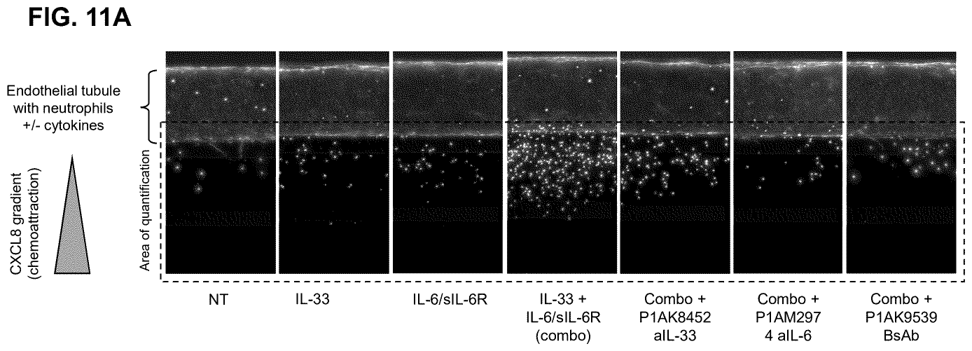

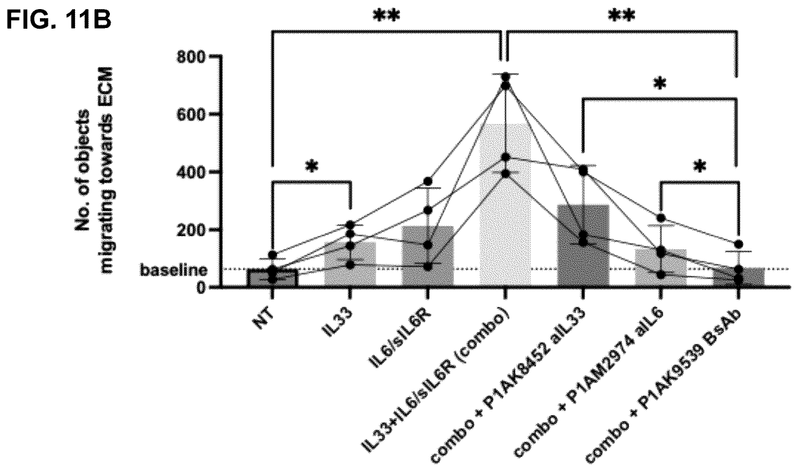

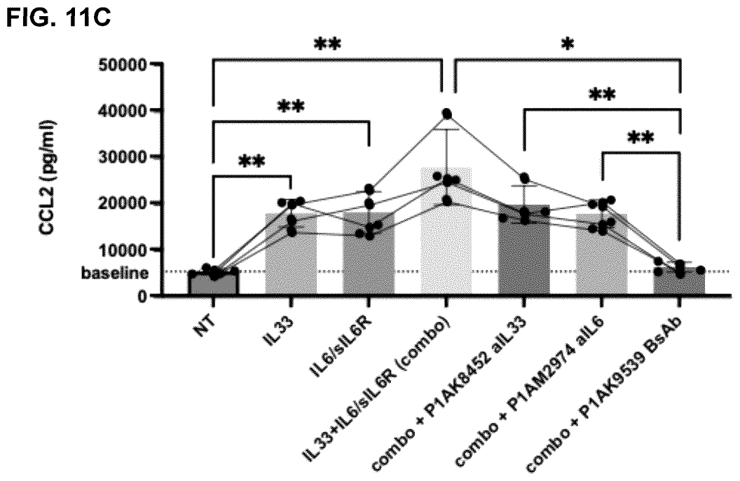

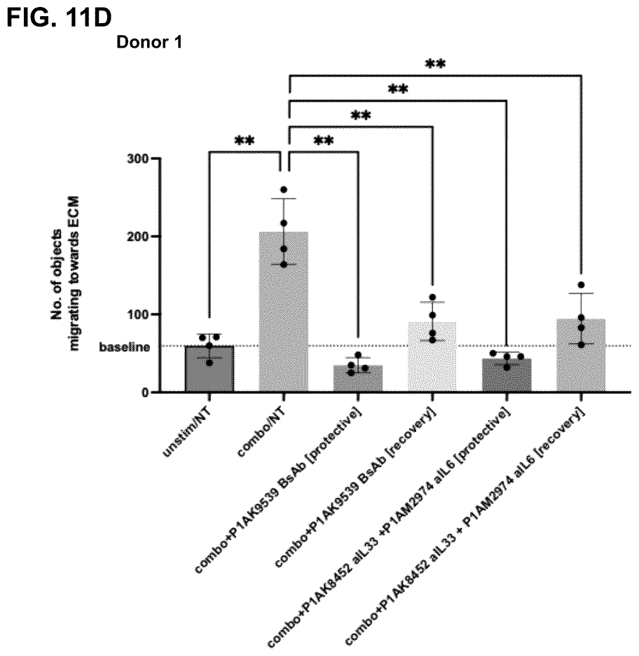

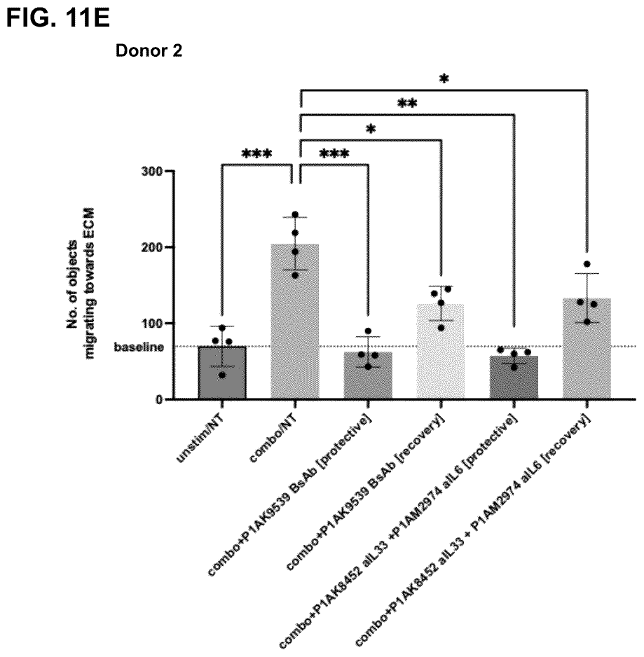

For FIG. 11B and FIG. 11C (Neutrophil Transmigration and CCL2 Levels), comparisons between treatment groups were conducted using a paired two-tailed t-test. This approach was utilized because the experiment involved multiple biological replicates of primary cells (n=4 independent neutrophil donors), with each donor's cells being exposed to all tested treatment conditions. The pairing accounts for intrinsic variability between individual donors. Statistical significance was defined as *=p<0.05, **=p<0.01.

For FIG. 11D and FIG. 11E (Protective vs. Recovery Setting), comparisons between the distinct intervention settings (protective vs. recovery) were performed using an unpaired two-tailed t-test. As these graphs illustrate results derived from a single neutrophil donor tested across different intervention conditions (n=1 neutrophil donor, with four technical replicates), the comparison assumes independence between the distinct treatment modalities tested. Statistical significance was defined as *=p<0.05, **=p<0.01, ***=p<0.001.

### Results

Given the pathways upregulated by the treatment of HUVECs with IL-33 and IL-6/sIL-6R (Example 6), we investigated the effect of the treatments on the transmigration of neutrophils through an endothelial layer. We utilized a microfluidic chip consisting of two independently accessible microfluidic channels on either side of an ECM scaffold. Endothelial cells were seeded in one of the channels, and once the endothelial cells had grown to form a tubule in the microfluidic channel and against the ECM gel, cytokine treatments were added for 24h. Following the treatment, neutrophils were added to the lumen of the endothelial tubule and were allowed to migrate through the endothelial layer and into the ECM towards a chemoattractant gradient of CXCL8. Both IL-33 and IL-6/sIL-6R treatments increased the transmigration of neutrophils; however, the effect of the combination of the two cytokine treatments was markedly increased (FIG. 11A, FIG. 11B).

To assess the redundancy of the two cytokines in inducing neutrophil transmigration, we attempted to prevent it by blocking either cytokine using a monospecific IL-33 antibody (P1AK8452, an IgG₁ antibody with the VH and VL sequences of SEQ ID NOs 43 and 44, respectively (and the PG LALA Fc mutations)) or a monospecific IL-6 antibody (P1AM2974, an IgG₁ antibody with the VH and VL sequences of SEQ ID NOs 4 and 8, respectively) or both cytokines by using the anti-IL-33/anti-IL-6 bispecific antibody P1AK9539. Inhibition of either cytokine by premixing the excess amounts of the respective antibody with the combination cytokine mix decreased, but was unable to completely prevent, the transmigration of neutrophils, demonstrating the complementary and overlapping effects of each cytokine to induce increased transmigration. Only the treatment with the bispecific antibody was able to block migration to baseline levels (FIG. 11A, FIG. 11B). The recruitment of monocytes and macrophages often follows an inflammatory response that causes neutrophilic infiltration, resulting in an accumulation of macrophages, which is a hallmark of COPD and emphysema. These cells release high levels of damaging enzymes, such as Matrix 20 Metalloproteinase-12 (MMP-12), which break down the elastic tissue of the lung, leading to the destruction of air sacs, characteristic of emphysema. One of the major chemoattractants of these cells is CCL2, whose main function is to attract cells that express its receptor, CCR2, which is predominantly found on monocytes (in the blood) and macrophages (in the tissue).

We thus investigated whether treatment with IL-33 and IL-6/sIL-6R affects the levels of CCL2 in the system. Both IL-33 and IL-6/sIL-6R, as well as the combined treatments, increased the levels of CCL2 in the system, and only the inhibition of both cytokines with the bispecific antibody P1AK9539 prevented the upregulation of this chemokine (FIG. 11C).

While premixing the blocking antibodies with the cytokine mix simulates patient treatment conditions in which excess free antibody circulates before exposure to the cytokines, i.e., protection, we sought to investigate whether inhibiting the cytokines after a short exposure, i.e., recovery, would help the endothelium recover and prevent further neutrophil migration.

We thus treated with cytokines IL-33, IL-6/sIL-6R, or the combination of the two, and a bispecific antibody or the combination of two monospecific antibodies in the protective setting, by premixing antibodies and cytokines, or the recovery setting by adding these antibodies 24 h after the cytokine treatment. Neutrophil migration was reduced in both the protective and recovery settings, with the former having a greater effect, reducing migration to below baseline levels in the two neutrophil donors tested (FIG. 11D, FIG. 11E).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating an inflammatory lung disease, particularly chronic obstructive pulmonary disease (COPD), in an individual, comprising administering to the individual a combination of a) an IL-33 inhibitor and b) an IL-6 inhibitor.

2-6. (canceled)

7. A bispecific antibody that binds IL-33 and IL-6, comprising a first antigen binding domain that binds to IL-6, and a second antigen binding domain that binds to IL-33, wherein the first antigen binding domain comprises a heavy chain variable region (VH) comprising the heavy chain complementarity determining region (HCDR) 1 of SEQ ID NO: 1, the HCDR 2 of SEQ ID NO: 2 and the HCDR 3 of SEQ ID NO: 3, and a light chain variable region (VL) comprising the light chain complementarity determining region (LCDR) 1 of SEQ ID NO: 5, the LCDR 2 of SEQ ID NO: 6 and the LCDR 3 of SEQ ID NO: 7, and wherein the second antigen binding domain comprises a heavy chain variable region (VH) comprising the heavy chain complementarity determining region (HCDR) 1 of SEQ ID NO: 9, the HCDR 2 of SEQ ID NO: 10 and the HCDR 3 of SEQ ID NO: 11, and a light chain variable region (VL) comprising the light chain complementarity determining region (LCDR) 1 of SEQ ID NO: 13, the LCDR 2 of SEQ ID NO: 14 and the LCDR 3 of SEQ ID NO: 15.

8. (canceled)

9. The bispecific antibody of claim 7, wherein the first antigen binding domain comprises a VH comprising an amino acid sequence having at least 95% sequence identity to the VH sequence of SEQ ID NO: 4, and a VL comprising an amino acid sequence having at least 95% sequence identity to the VL sequence of SEQ ID NO: 8, and/or wherein the second antigen binding domain comprises a VH comprising an amino acid sequence having at least 95% sequence identity to the VH sequence of SEQ ID NO: 12, and a VL comprising an amino acid sequence having at least 95% to the VL sequence of SEQ ID NO: 16.

10. (canceled)

11. The bispecific antibody of claim 7, wherein the first antigen binding domain is a Fab molecule wherein the variable domains VL and VH of the Fab light chain and the Fab heavy chain are replaced by each other, and the second antigen binding domain is a conventional Fab molecule.

12. The bispecific antibody of claim 11, wherein: (i) in the constant domain CL of the second antigen binding domain the amino acid at position 124 is substituted independently by lysine (K), arginine (R) or histidine (H) and the amino acid at position 123 is substituted independently by lysine (K), arginine (R) or histidine (H), wherein the amino acid numbering in the CL is according to Kabat; and (ii) in the constant domain CH1 of the second antigen binding domain the amino acid at position 147 is substituted independently by glutamic acid (E), or aspartic acid (D) and the amino acid at position 213 is substituted independently by glutamic acid (E), or aspartic acid (D), wherein the amino acid numbering in the CH1 is according to Kabat EU index.

13. The bispecific antibody of claim 7, comprising an Fc domain composed of a first subunit and a second subunit.

14. The bispecific antibody of claim 13, wherein the Fc domain is a human IgG₁ Fc domain.

15. The bispecific antibody of claim 13, wherein the Fc domain comprises a modification promoting the association of the first subunit and the second subunit of the Fc domain.

16. The bispecific antibody of claim 13, wherein the Fc domain comprises a modification that reduces the binding affinity to an Fc receptor and/or effector function of the antibody, as compared to a corresponding antibody comprising an Fc domain without such modification.

17. The bispecific antibody of claim 13, wherein the Fc domain comprises a modification that increases the binding affinity to an FcRn receptor and/or the serum half-life of the antibody, as compared to a corresponding antibody comprising an Fc domain without such modification.

18. The bispecific antibody of claim 13, wherein the first antigen binding domain and the second antigen binding domain are each a Fab molecule and are each fused at the C-terminus of the Fab heavy chain to the N-terminus of one of the subunits of the Fc domain.

19. (canceled)

20. An isolated polynucleotide encoding the bispecific antibody of claim 7.

21. A host cell comprising the isolated polynucleotide of claim 20.

22. A method of producing a bispecific antibody that binds IL-33 and IL-6, comprising culturing the host cell of claim 21 under conditions suitable for the expression of the bispecific antibody.

23. A bispecific antibody that binds IL-33 and IL-6 produced by the method of claim 22.

24. A pharmaceutical composition comprising the bispecific antibody of claim 7 and a pharmaceutically acceptable carrier.

25-28. (canceled)

29. A method of treating a disease, particularly an inflammatory lung disease in an individual, comprising administering to the individual the bispecific antibody of claim 7.

30-31. (canceled)

32. A kit comprising a first medicament comprising a IL-33 inhibitor and a second medicament comprising a IL-6 inhibitor, and optionally further comprising a package insert comprising instructions for administration of the first medicament in combination with the second medicament for treating an inflammatory lung disease in an individual according to the method of claim 1.

33. (canceled)

34. A bispecific antibody that binds IL-33 and IL-6, comprising:
(a) a first polypeptide comprising the amino acid sequence of SEQ ID NO: 17;
(b) a second polypeptide comprising the amino acid sequence of SEQ ID NO: 20;
(c) a third polypeptide comprising the amino acid sequence of SEQ ID NO: 23;
(d) a fourth polypeptide comprising the amino acid sequence of SEQ ID NO: 24.

35. The bispecific antibody of claim 7, wherein the first antigen binding domain comprises a VH comprising the amino acid sequence of SEQ ID NO: 4 and a VL comprising the amino acid sequence of SEQ ID NO: 8, and the second antigen binding domain comprises a VH comprising the amino acid sequence of SEQ ID NO: 12 and a VL comprising the amino acid sequence of SEQ ID NO: 16.

36. The bispecific antibody of claim 14, wherein the human IgG1 Fc domain comprises:
(i) in the first subunit the amino acid substitutions S354C and T366W, and in the second subunit the amino acid substitutions Y349C, T366S, L368A and Y407V;
(ii) in each of the first subunit and the second subunit the amino acid substitutions L234A, L235A and P329G; and
(iii) in each of the first subunit and the second subunit the amino acid substitutions M252Y, S254T and T256E, wherein the amino acid numbering is according to Kabat EU index.
